SEQUENCE LISTING (6 sequences)

SEQ ID NO: 1 - RNA, 20 nt, Artificial Sequence
  Description of Artificial Sequence Synthetic oligonucleotide
caaccagcuaagacacugcc

SEQ ID NO: 2 - RNA, 22 nt, Artificial Sequence
  Description of Artificial Sequence Synthetic oligonucleotide
uggcagugucuuagcugguugu

SEQ ID NO: 3 - RNA, 21 nt, Artificial Sequence
  Description of Artificial Sequence Synthetic oligonucleotide
gaagugcucguccucguccuu

SEQ ID NO: 4 - RNA, 21 nt, Artificial Sequence
  Description of Artificial Sequence Synthetic oligonucleotide
uucuucacgagcaggagcagg

SEQ ID NO: 5 - RNA, 21 nt, Artificial Sequence
  Description of Artificial Sequence Synthetic oligonucleotide
caaccagcuaagacacugccu

SEQ ID NO: 6 - RNA, 21 nt, Artificial Sequence
  Description of Artificial Sequence Synthetic oligonucleotide
uggcagugucuuagcugguug

(19) United States
(12) Patent Application Publication
(10) Pub. No.: US 2019/0298843 A1
(43) Pub. Date: Oct. 3, 2019

(54) LIGAND IONOPHORE CONJUGATES

(71) Applicant: PURDUE RESEARCH FOUNDATION, West Lafayette, IN (US)

(72) Inventors: Philip Stewart LOW, West Lafayette, IN (US); Andrea L. KASINSKI, West Lafayette, IN (US); Loganathan RANGASAMY, West Lafayette, IN (US); Venkatesh CHELVAM, West Lafayette, IN (US)

(21) Appl. No.: 16/413,940

(22) Filed: May 16, 2019

Related U.S. Application Data
(63) Continuation-in-part of application No. PCT/US2017/061997, filed on Nov. 16, 2017.
Application No. PCT/US2017/061997 is a continuation-in-part of application No. 15/572,985, filed on Nov. 9, 2017.
(60) Provisional application No. 62/422,922, filed on Nov. 16, 2016.
(60) Provisional application No. 62/478,063, filed on Mar. 29, 2017.
(60) Provisional application No. 62/159,659, filed on May 11, 2015.

(51) Int. Cl. A61K 47/55; A61K 49/00; A61K 47/54; A61P 35/00; A61K 31/7105

(52) U.S. Cl. CPC A61K 47/551; A61K 49/0032; A61K 49/0052; A61K 31/7105; A61K 47/545; A61P 35/00; A61K 47/549

(57) ABSTRACT

The invention described herein pertains to ligand-ionophore conjugates, that may also comprise a linked therapeutic agent or imaging agent, and pharmaceutical compositions containing the conjugates. Also described are methods of using the conjugates for increasing the endosomal accumulation and escape of a therapeutic agent, or an imaging agent.

### CROSS REFERENCE TO RELATED APPLICATIONS

This patent application is a continuation-in-part of, and claims priority to, PCT International Application No. PCT/US2017/061997, filed Nov. 16, 2017, which claims priority under 35 U.S.C. § 119(e) to U.S. Provisional Application Ser. No. 62/422,922, filed Nov. 16, 2016 and U.S. Provisional Application Ser. No. 62/478,063, filed Mar. 29, 2017; and this patent application is a continuation-in-part of, and claims priority to under 35 U.S.C. § 120 to U.S. application Ser. No. 15/572,985, filed Nov. 9, 2017, which claims priority to PCT International Application No. PCT/US2016/031738, filed May 11, 2016, which claims priority under 35 U.S.C. § 119(e) to U.S. Provisional Application Ser. No. 62/159,659, filed May 11, 2015, the disclosures of each of which is incorporated herein by reference in their entirety.

### GOVERNMENT RIGHTS

This invention was made with government support awarded by the National Institutes of Health and the National Cancer Institute through grant P30 CA023168. The U.S. government has certain rights in the invention.

### TECHNICAL FIELD

The invention described herein pertains to ligand ionophore conjugates, which may also comprise a linked therapeutic agent or a linked imaging agent, and pharmaceutical compositions containing the conjugates. Also described are methods of using the described conjugates for increasing the endosomal accumulation and escape of a therapeutic agent, or an imaging agent, that is internalized by endocytosis or an analogous process. Also described is the delivery of microRNAs to tumor tissues by direct attachment of microRNAs to folate, (FolamiR), which mediates delivery of the conjugated microRNA into cells that overexpress folate receptor.

### BACKGROUND

Many diseases can be treated with a drug or a biologic agent (illustrative examples of biologic agents include nucleotides, e.g. siRNA, miRNA and the like; amino acids, including synthetic amino acids not occurring in nature; proteins, including enzymes, peptides, aptamers, antigens and the like; and antibodies, e.g. glycoproteins, immunoglobulins and the like). These drugs or biologics can be delivered into their target cells with targeting ligands, e.g. a folate receptor binding ligand, but their efficacy can be inhibited by an inability of the drug or biologic agent to be released from the endosome, for example, after folate-mediated endocytosis. Therefore discovery of new methods for “endosomal release” of trapped cargo into the cytoplasm would be useful for achieving increased efficacy of targeted drugs or biologics. It has been discovered that endosomal release can be facilitated by use of ligand ionophore conjugates to create osmotic pressure to rupture the endosomes containing the cargo using known ionophores that have low toxicity to healthy tissues. Without being bound by theory it is believed that nigericin, an ionophore and antiporter that couples efflux of H⁺ ions to influx of K⁺ ions, if delivered into cells, causes an osmotic imbalance inside endosomes leading to a swelling and/or disruption of the endosome and the release of the endosomal contents into cytoplasm. It will be appreciated that other K⁺ ionophores like salinomycin that transport potassium ions can also be employed for endosomal release.

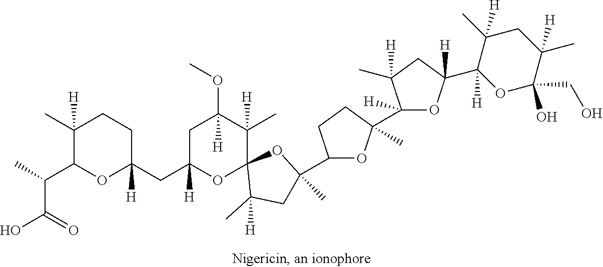

In order to induce swelling of an endosome, an osmotically active ion can enter the endosome and promote the accompanying osmotically driven influx of water. This influx of water should force the endosome to enlarge, ultimately leading to its rupture. However, if the influx of the osmotically active ion is accompanied by the efflux of another osmotically active ion, no net change in water flow will occur and the endosome will not expand. Thus, for endosome swelling to occur, an osmotically active ion (e.g., Na⁺, K⁺, Li⁺, Ca⁺⁺, Mg⁺⁺) should enter the endosome in exchange for H⁺, which is the only osmotically inactive cation in nature. Moreover, because the only osmotically active ion that will flow spontaneously down its concentration gradient into an endosome is K⁺, an ionophore that is useful to lead to swelling of an endosome is an ionophore that can exchange K⁺ ions for H⁺ ions.

The Na⁺/H⁺ exchanger (antiporter) is a natural endosomal transporter whose function is to modify endosomal pH. It can work against a K⁺ ionophore-induced endosomal swelling by moving sodium ions out of the endosome in exchange for H⁺, leading to endosome shrinkage. Thus, the action of a K⁺ ionophore might be reduced by a naturally occurring Na⁺/H⁺ exchanger (antiporter), but augmented by the simultaneous addition of an inhibitor of the Na⁺/H⁺ exchanger such as amiloride, or HOE 694, or the like.

Folate receptors are over expressed on the cell membrane of many human cancers like ovarian, lung, breast, endometrium, brain, kidney and colon cancer and in activated macrophages which are responsible for inflammatory diseases like rheumatoid arthritis, artherosclerosis, osteoarthritis, diabetes, psoriasis etc. Folic acid has high binding affinity (K_{d}=10-10M) for folate receptors and can deliver releasable cargo to folate receptors in a selective manner avoiding off-site toxicity. Ligands bound to these receptors become part of the endosome that forms after the membrane invaginates into caveolae, internalizes and separates from the surface.

Prostate specific membrane antigen (PSMA) is a cell surface protein that is internalized in a process analogous to the endocytosis observed with cell surface receptors, such as folate receptors. It has been established that biologically active compounds that are conjugated via a linker to ligands capable of binding to PSMA may be useful in the imaging, diagnosis, and/or treatment of prostate cancer, and related diseases that involve pathogenic cell populations expressing or over-expressing PSMA. PSMA is over-expressed in malignant prostate tissues when compared to other organs in the human body such as kidney, proximal small intestine, and salivary glands. Although PSMA is expressed in brain, that expression is minimal, and most ligands of PSMA are polar and are not capable of penetrating the blood brain barrier. Unlike many other membrane-bound proteins, PSMA undergoes rapid internalization into the cell in a similar fashion to cell surface receptors like folate receptors. PSMA is internalized through clathrin-coated pits and subsequently can either recycle to the cell surface or be retained inside an endosome which progressively develops into a lysosome.

Even though a drug cargo delivered to a receptor capable of endocytosis, or an analogous process, is delivered selectively to the diseased cells, the path of delivered cargo to the cytoplasm or the nucleus can be blocked completely or partially by the invaginated plasma membrane called the ‘endosome’. Higher molecular weight agents, such as peptides, siRNAs, antisense oligonucleotides, proteins, aptamers, oligosaccarides and polysaccarides cannot escape endosomes once they have been internalized via a ligand-targeted endocytosis pathway. Thus the trapped cargo stays in the endosome and finally decomposes to smaller fragments by the action of acids and enzymes present in the endosome before being released in inactive form. The conjugates of the invention increase both the endosomal accumulation and escape of a therapeutic agent, or an imaging agent in targeted cells.

### SUMMARY

In some embodiments, the present disclosure provides a targeted microRNA delivery system comprising a conjugate of covalently linked folate and a microRNA or its mimics, and a pharmaceutically acceptable carrier, diluent, or recipient.

Several embodiments of the invention are described in the following clauses:

1. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and/or a therapeutic agent (TA) comprising an siRNA, an iRNA, or a microRNA;

wherein (L) optionally comprises at least one releasable linker; (B) is covalently linked to (L); and each of (A) and/or (TA) is covalently linked to (L).

1a. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and

a therapeutic agent comprising an siRNA, an iRNA, or a microRNA;

wherein (L) comprises at least one releasable linker; (B) is covalently linked to (L); and each (A) is covalently linked to (L).

2. The conjugate of clause 1 or 1a, or a pharmaceutically acceptable salt thereof, wherein (L) comprises at least one releasable linker.

3. The conjugate of clause 1 or 1a, or a pharmaceutically acceptable salt thereof, wherein the therapeutic agent (TA) is covalently linked to (L).

4. The conjugate of clause 1 or 1a, or a pharmaceutically acceptable salt thereof, wherein the therapeutic agent (TA) comprises an siRNA.

5. The conjugate of clause 1 or 1a, or a pharmaceutically acceptable salt thereof, wherein the therapeutic agent (TA) comprises an iRNA.

6. The conjugate of clause 1 or 1a, or a pharmaceutically acceptable salt thereof, wherein the therapeutic agent (TA) comprises a microRNA.

7. The conjugate of any one of the preceding clauses, or a pharmaceutically acceptable salt thereof, wherein (B) is a folate.

8. The conjugate of any one of the preceding clauses 1 to 6, or a pharmaceutically acceptable salt thereof, wherein (B) is a PSMA binding ligand.

9. The conjugate of any one of the preceding clauses, or a pharmaceutically acceptable salt thereof, wherein (A) is an inhibitor of the Na⁺/H⁺ exchanger.

10. The conjugate of clause 9, or a pharmaceutically acceptable salt thereof, wherein the ionophore (A) comprises nigericin or salinomycin.

11. The conjugate of any one of the preceding clauses, or a pharmaceutically acceptable salt thereof, wherein (L) comprises a chain of about 7 to about 45 atoms.

12. The conjugate of clause 1 or 1a, having a formula selected from the group consisting of

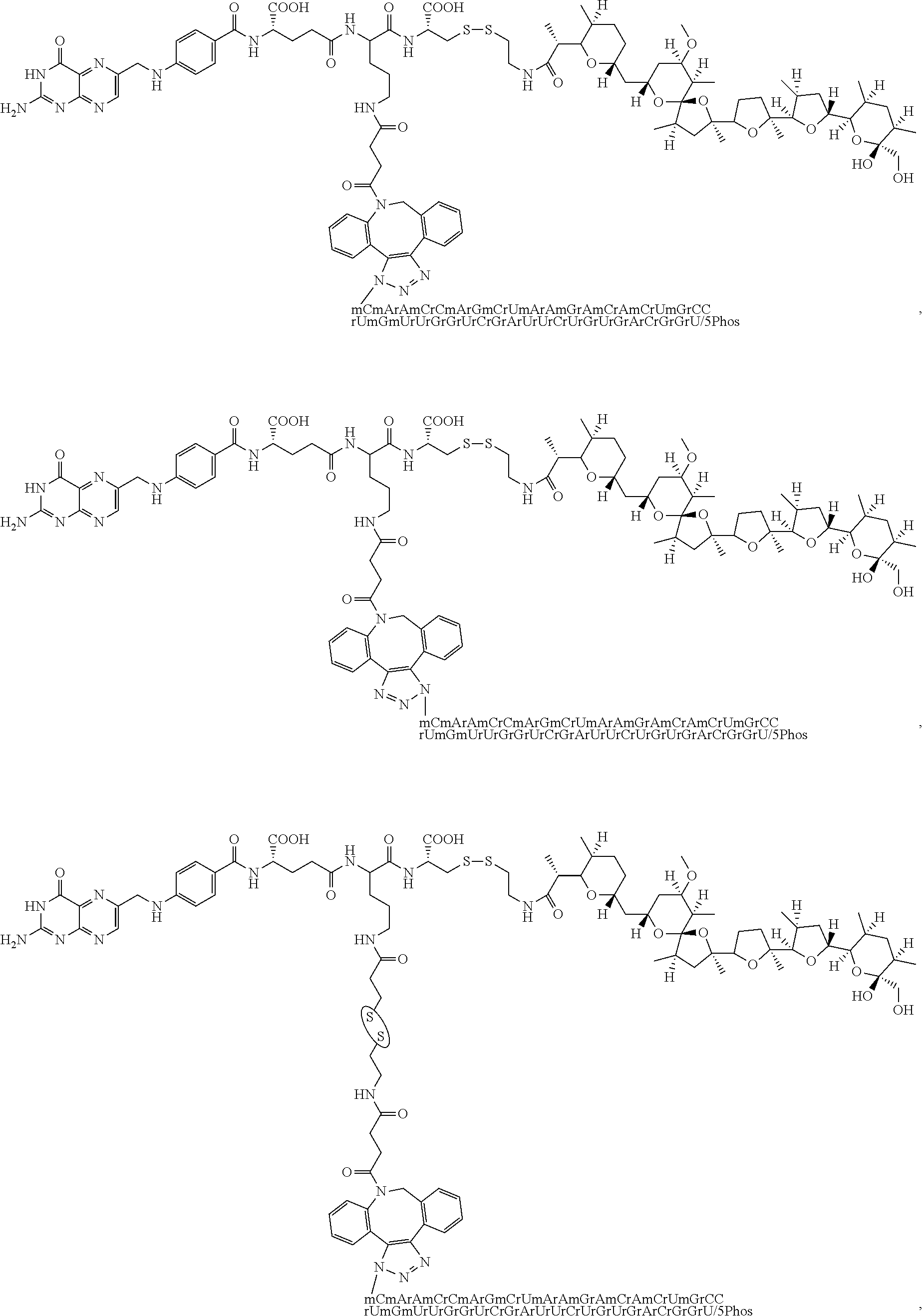

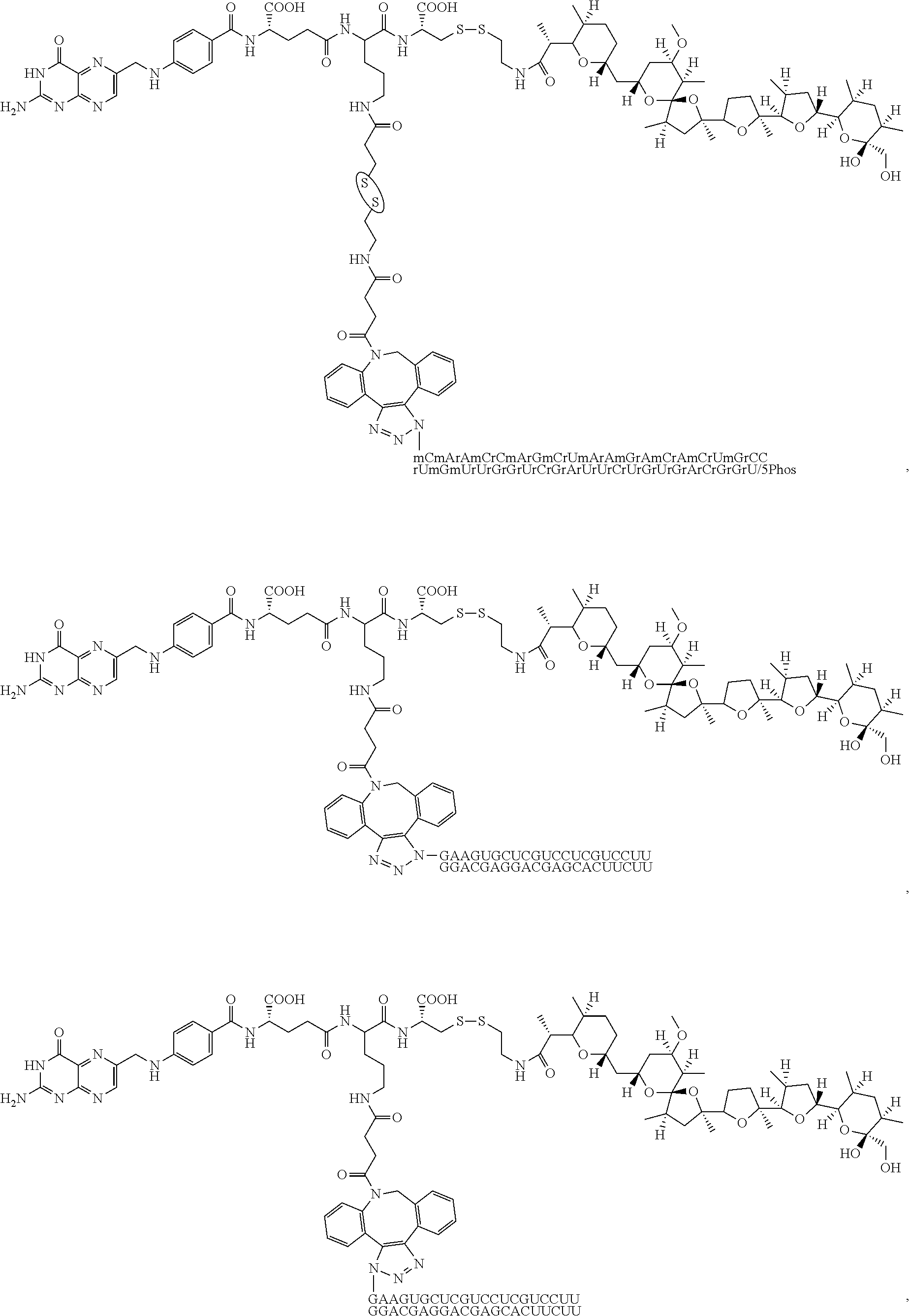

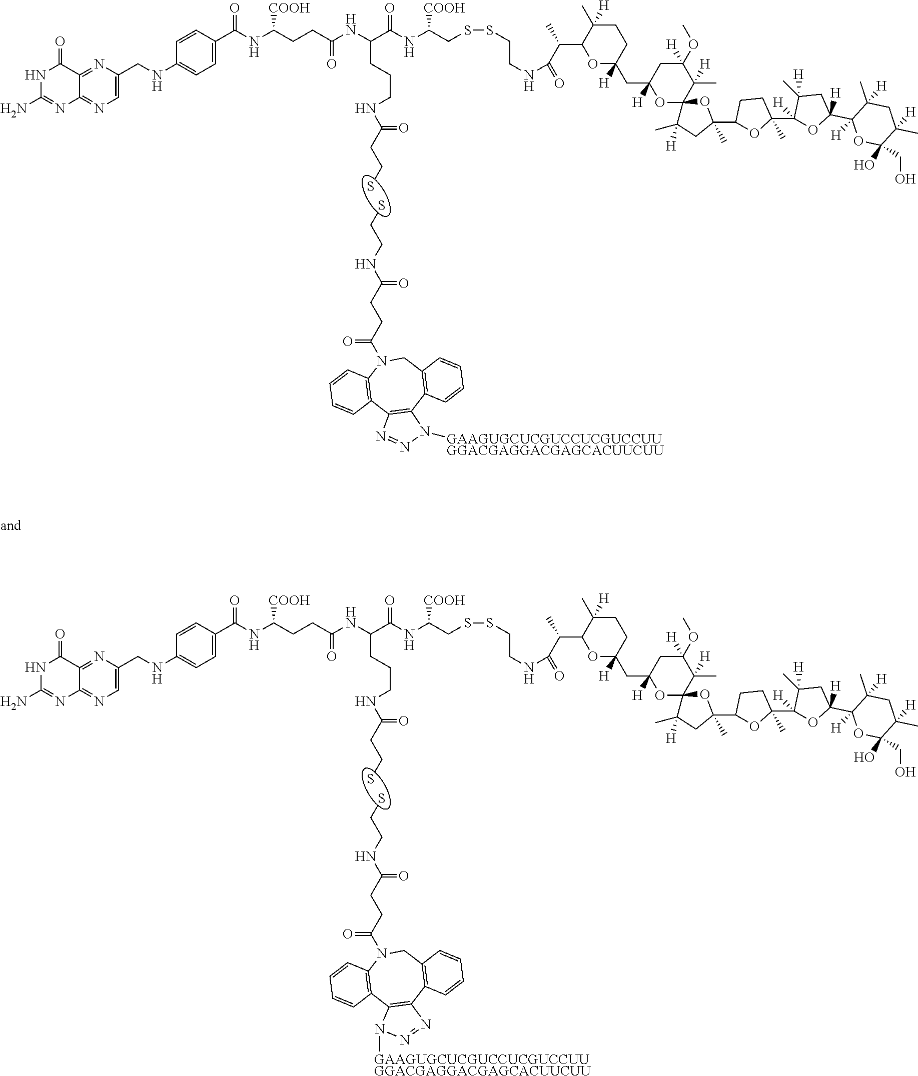

or a pharmaceutically acceptable salt thereof.

13. The conjugate of clause 1 having a formula

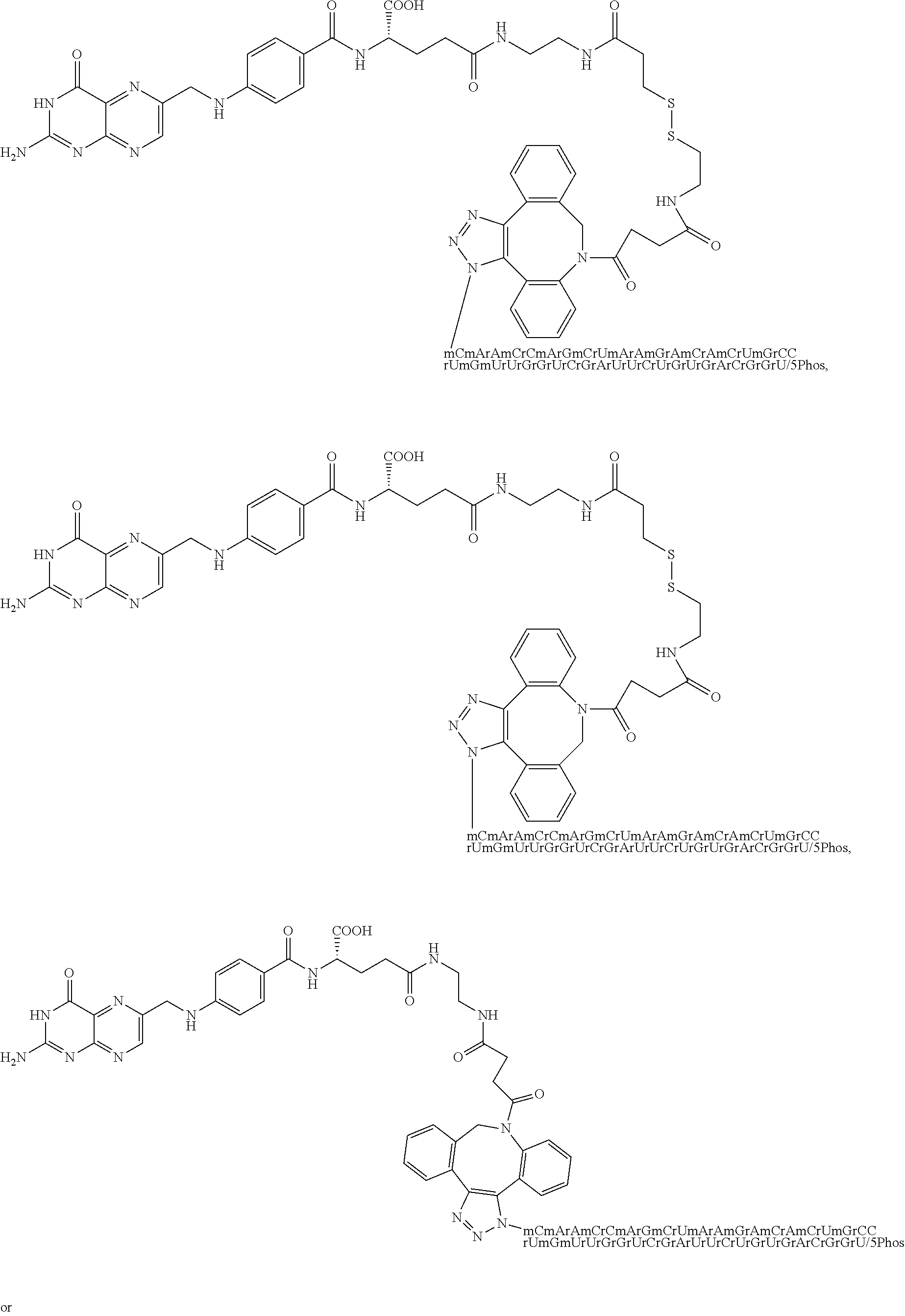

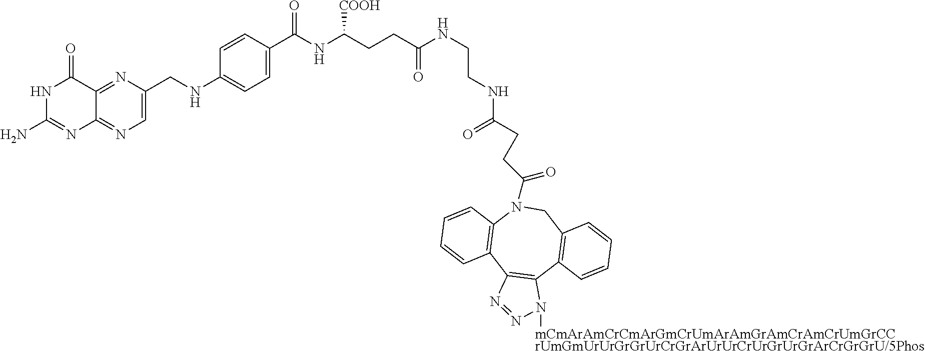

or a pharmaceutically acceptable salt thereof.

14. A pharmaceutical composition comprising at least one conjugate of any one of clauses 1 to 13, or a pharmaceutically acceptable salt thereof, and at least one pharmaceutically acceptable carrier or excipient.

15. A pharmaceutical composition comprising at least one conjugate of any one of clauses 1 to 13, or a pharmaceutically acceptable salt thereof, and an additional therapeutic agent.

16. A method of increasing the endosomal accumulation and escape of a therapeutic agent or an imaging agent, the method comprising the step of administering with the therapeutic agent or the imaging agent an effective amount of the conjugate of any one of clauses 1 to 13, or a pharmaceutically acceptable salt thereof.

17. The method of clause 16 wherein the therapeutic agent or the imaging agent is targeted to a cancer.

18. The method of clause 17 wherein the cancer is selected from the group consisting of ovarian, lung, breast, endometrial, brain, kidney, prostate, and colon cancer.

19. The method of clause 16 wherein the therapeutic agent is targeted to a site of inflammation.

20. The method of clause 19 wherein the site of inflammation is caused by an inflammatory disease selected from the group consisting of rheumatoid arthritis, osteoarthritis, atherosclerosis, diabetes, graft-versus-host disease, multiple sclerosis, osteomyelitis, psoriasis, Crohn's disease, Sjögren's syndrome, lupus erythematosus, and ulcerative colitis.

21. A conjugate, or a pharmaceutically acceptable salt thereof, comprising:

a ligand (B) targeted to a cell-surface receptor;

one or more linkers (L);

one or more of an ionophore (A) which couples efflux of protons (H⁺ ions) to influx of potassium ions (K⁺ ions); an RNA selected from an siRNA, an iRNA, and a microRNA; or an imaging agent (IA);

wherein (L) comprises at least one releasable linker; (B) is covalently linked to (L); and each of (A), the RNA and/or (IA) is covalently linked to (L).

22. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and

a fluorescent dye comprising Cy5;

wherein (L) comprises at least one releasable linker; (B) is covalently linked to (L); and each (A) is covalently linked to (L).

23. The conjugate of clause 21 or 22, having a formula

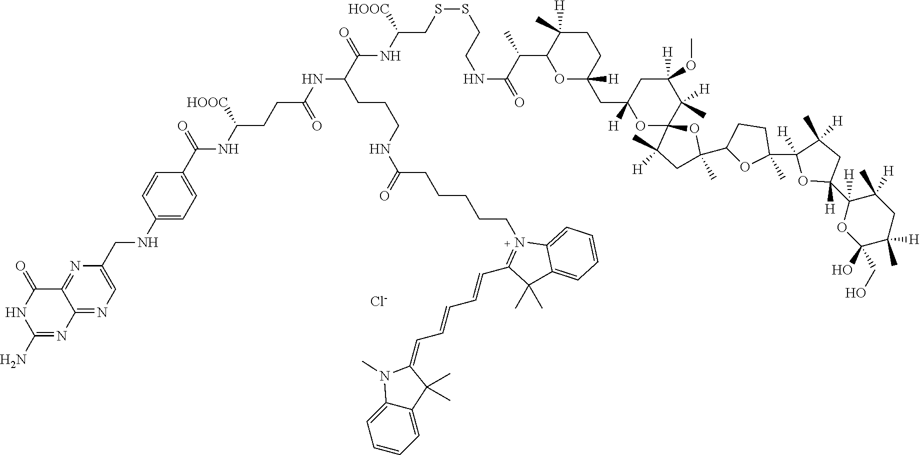

or a pharmaceutically acceptable salt thereof.

23. The conjugate of clause 21, having a formula

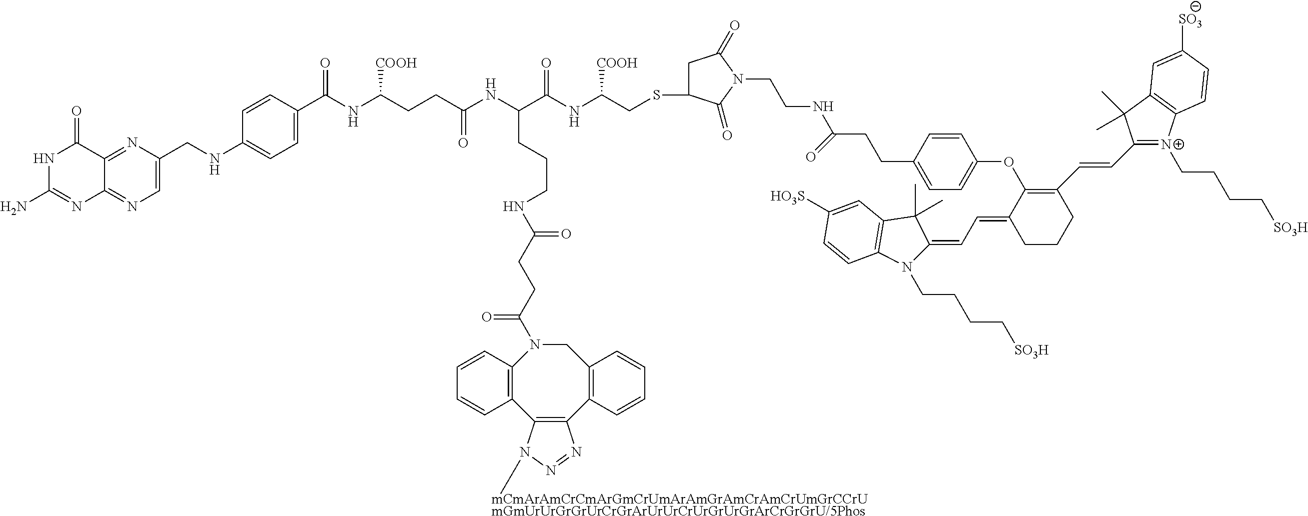

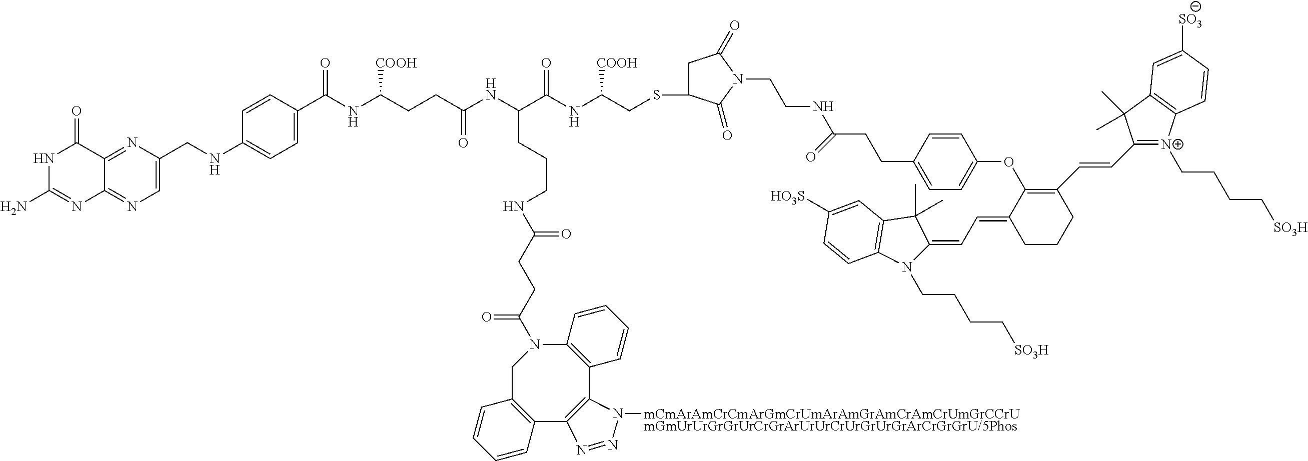

or a pharmaceutically acceptable salt thereof.

### BRIEF DESCRIPTION OF THE DRAWINGS

FIG. 1 shows targeted silencing of miR-34a Renilla sensor using Folate conjugate in vitro. Data points were normalized to Folate-NC (negative control: scrambled miRNA) for each time point. (Fol-DB-miR34a: Folate-DBCO-miR34a; Fol-SS-Nig miR34a: Folate-ss-DBCO-nigericin-miR34a; Fol-DB-Nig miR34a: Folate-DBCO-nigericin-miR34a)

FIG. 2 shows a plot of luciferase relative light units normalized to negative control versus time in hours. The data show that Fol-Nig-siLuc induces early luciferase knockdown in MDA-MB-231 cells. Luciferase activity levels from MDA-MB-231 sensor cells normalized to Fol-negative control (Fol-NC: Folate-DBCO-negative control (scramble RNA) or Fol-Nig-NC: Folate-nigericin-DBCO-negative control (scramble RNA)). Mean±S.D., technical replicates=3, n=3, ** P<0.01. The arrow indicates replacement of media with a new dose of folate conjugates (50 nM). Fol-SiLuc2: Folate-DBCO-siLuc2; Fol-Nig-SiLuc2: Folate-nigericin-DBCO-siLuc2.

FIGS. 3A-B shows live cell images of MDA-MB-231 cells stably expressing Rab5B-GFP treated with Folate-Cy5 (50 nM) 3 h post treatment.

FIGS. 4A-B shows live cell images of MDA-MB-231 cells stably expressing Rab5B-GFP treated with Folate-nigericin-Cy5 (50 nM) 3 h post treatment.

FIGS. 5A-F show specificity of FolamiR uptake in cells in culture. FIG. 5A shows a proposed mechanism of action of FolamiRs. FIG. 5B shows structures of FolamiR-34a conjugates bearing an unreleasable ligand—FolamiR-34a, a releasable ligand—FolamiR-SS-34a (disulfide bond shown in red), and a miR-34a conjugate bearing an unreleasable folate ligand and a NIR moiety (shown in green)—NIR-FolamiR-34a. Folate moiety is shown in blue and miRNA in red. FIG. 5C shows the identification of folate receptor α (FRα) in FR positive MDA-MB-231 breast cancer cells and in FR negative A549 lung cancer cells. Histograms represent overlaid flow cytometry data as a percentage of unstained (A), FRα (C) and isotype control (B) stained cells. FIG. 5D shows NIR-FolamiR-34a uptake in FR positive MDA-MB-231 breast cancer cells compared to FR negative A549 lung cancer cells. Histograms represent overlaid flow cytometry data as a percentage of unstained (denoted with an A), and NIR-FolamiR-34a (50 nM) stained cells (denoted with a B). FIG. 5E shows folate-fluorescein isothiocyanate (Fol-FITC) uptake in FR positive MDA-MB-231 breast cancer cells compared to FR negative A549 lung cancer cells. Scale bar: 50 m. FIG. 5F shows targeted silencing of miR-34a Renilla sensor using FolamiR in vitro.

FIGS. 6A-E show cellular responses to FolamiRs. FIG. 6A shows targeted silencing of miR-34a Renilla sensor using FolamiR in vitro. Data points were normalized to FolamiR-NC (negative control: scrambled miRNA) for each time point. FIG. 6B shows proliferation and survival of MDA-MB-231 cancer cells as a function of FolamiR treatment (50 nM). Data points were normalized to FolamiR-NC for each time point. Error bars represent mean±s.d. Each experiment corresponds to n=3 with at least 4 technical replicates per treatment. FIG. 6C shows dose response of MDA-MB-231 to FolamiR-34a. Renilla values were measured 96 hours post treatment. Data points were normalized to FolamiR-NC. Error bars represent mean±s.d. Each experiment corresponds to n=3 with at least 4 technical replicates per treatment, statistical analysis performed with a one-way ANOVA with post hoc Bonferroni correction, (**, P<0.01; ****, P<0.0001). FIG. 6D shows displacement of NIR-FolamiR-34a binding from human MDA-MB-231 cells (50 nM, 4° C.) with increasing concentrations of folate glucosamine conjugate. Histograms represent overlaid flow cytometry data as a percentage of unstained, and NIR-FolamiR-34a stained cells. FIG. 6E shows in vitro FolamiR-34a competition assay.

FIGS. 7A-G demonstrates that FolamiR-34a inhibits the growth of MDA-MB-231 tumors. FIG. 7A shows a representative live imaging of female Nu/Nu congenic mice implanted with MDA-MB-231 sensor xenografts following intravenous injection of 5 nmol of NIR-FolamiR-34a, NIR-FolamiR-SS-34a or NIR-FolamiR-NC. Left side depicts fluorescent distribution and right side shows miR-34a renilla sensor signal. FIG. 7B shows effects of NIR-FolamiR-34a delivery on MDA-MB-231 Renilla sensor activity; all data normalized to the Renilla signal at day 0, data are shown as mean±s.e.m., with n=3, statistical analysis performed with a two-way ANOVA with post hoc Bonferroni correction, (**, P<0.01). FIG. 7C shows gross images of MB-231 breast tumors and whole body organs visualized for fluorescence (T, tumor; Int, intestines; S, spleen; K, kidneys; Lv, liver; HLu, heart lung). FIG. 7D shows miR-34a levels from excised MDA-MB-231 tumors measured by qRT-PCR at 72 hours post injection with NIR-FolamiR conjugates (n=3; error bars represent mean±s.d, statistical analysis performed with one-way ANOVA with post hoc Bonferroni correction, **, P<0.01). FIG. 7E shows NIR epifluorescence quantification from live animals. Female Nu/Nu congenic mice were implanted with A549 cells on the left shoulder and MDA-MB-231 sensor xenografts on the right shoulder and live imaging was conducted following intravenous injection of 5 nmol of NIR-FolamiR-34a in the presence (right) or absence (left) of ≥100-fold molar excess of folate-glucosamine (n=3 per group). Error bars represent mean±s.d., statistical analysis performed with a one-way ANOVA, **, P<0.01; ***, P<0.001; ****, P<0.0001). FIG. 7F shows fluorescent distribution of procured organs and tumors from FIG. 7E: A549: FR negative tumor; MB231 (MDA-MB-231): FR positive tumor; Int, intestines; S, spleen; K, kidneys; Lv, liver; HLu, heart lung). FIG. 7G shows tumor size following FolamiR-34a treatment (n=5, error bars represent mean±s.e.m., statistical analysis performed with a two-way ANOVA, **, P<0.01; ***, P<0.001). Arrows represent treatment times (1 nmol i.v. injection). Tumors were measured with a vernier caliper and tumor volume was calculated by: volume (mm³)=width×(length²)×2⁻¹.

FIGS. 8A-D shows that Murine Kras^{LSL-G12D/+}; p53^{flx/flx} lung adenocarcinomas express FR (folate receptor). FIG. 8A shows fluorescent imaging ligand OTL38 (On Target Laboratories, LLC., West Lafayette, Ind.; folate receptor-alpha (FRα)-targeting ligand conjugated to a fluorescent near infrared (NIR) dye) is preferentially retained in lung tumors and cleared from normal healthy tissue. ^{KrasLSL-G12D/+}; p53^{Flox/Flox} mice were injected with 5 nmol of OTL38 eight weeks after tumor induction and sacrificed 24 hours post injection. A noninduced healthy mouse was used as control. RC, right caudal lobe; RM, right medial lobe; RA, right accessory lobe, RCr, right cranial lobe; L, left lobe. FIG. 8B shows a histological view of right lobe of lungs from mice treated with OTL38. Left, NIR imaging of whole organ view with matching H&E stained slide. Right, high magnification images of tumorous and healthy tissue. H&E images represent the type of tissue shown in bright field and near infrared images. Scale bar: 50 m; Inset: 20 μm. Numbered boxes shown on low magnification images correlate with numbers on high magnification images. FIG. 8C shows whole organ view of lung lobes from mice treated with OTL38 (5 nmoles) in the presence or absence of ≥100-fold molar excess of folate-glucosamine (n=3 per group). FIG. 8D shows representative histological views of tissues from FIG. 8C. FIG. 8B, D shows low magnification H&E stained tissues with their corresponding high magnification images of tumorous tissue. On left, whole organ NIR image view with matching high magnification NIR images. H&E images represent the type of tissue shown in near infrared images. Squares in low magnification H&E image correlate with images shown in high magnification. 10 Scale bar: 20 m.

FIGS. 9A-F demonstrate that targeted replacement of miR-34a via FolamiR has beneficial effects in a murine model of lung adenocarcinoma. FIG. 9A shows MRI-measured tumor burden following FolamiR-34a treatment (n=4 or 5, error bars represent mean±s.e.m., statistical analysis performed with a two-way ANOVA and Bonferroni post hoc tests, **, P<0.01). Arrows represent treatment times (1 nmol intravenous injection; total 10). FIG. 9B shows representative MRI images and 3D renders of mice treated with FolamiRs during (day 8) and at the end of treatment period (day 29). FIG. 9C shows Tumor/whole lung ratios at the indicated times showing the percentage of lung volume occupied by tumors. Error bars represent mean±s.d., statistical analysis performed with a one-way ANOVA, *, P<0.05. FIG. 9D shows representative H&E stained tissue of the left lobe of animals from each treatment group. Scale bars=1 mm. FIG. 9E shows overall tumor burden is calculated from total tumor area averaged from three sections obtained from each treated animal relative to the total area of the lung. (Bars indicate the median, FolamiR-NC: n=4, FolamiR-34a: n=5; unpaired t-test). FIG. 9F shows miR-34 target genes, Met, Myc, and Bcl-2 were evaluated by qRT-PCR, normalized to Actin, and graphed relative to FolamiR-NC treated tumors. (Bars indicate the median, unpaired t-test: * P<0.05).

FIG. 10 shows miR34a Renilla sensor response to miRNA mimic transfection. MDA-MB-231 breast cancer cells and A549 lung cancer cells transiently expressing a miR-34a Renilla sensor were used to monitor miR-34a delivery and activity. MDA-MB-231 and A549 cells were transfected with 50 nM of miR-34a mimic using Lipofectamine RNAimax (Life Technologies) and Renilla signal was measured 96 hours post treatment.

FIGS. 11A-B show evaluation of MDA-MB-231 miR-34a sensor cells. FIG. 11A shows miR-34a sensor specificity and silencing activity of endogenous miR-34a in MDA-MB-231 cells. Error bars represent the mean±s.d., experiments were performed in triplicate. FIG. 11B shows selection of MB-231 clones based on renilla activity. Renilla readings were performed using 1×10⁴ cells per clone and renilla levels were measured using the Renilla Glo Luciferase Kit (Promega).

FIG. 12 shows tumor growth response to increasing doses of FolamiR34a. Tumor size following FolamiR-34a treatment (n=5, error bars represent mean±s.e.m., statistical analysis performed with a two-way ANOVA, ***, P<0.001). Arrows represent treatment times (intravenous injection). Tumors were measured with a vernier caliper and tumor volume was calculated by: volume (mm³)=width×(length²)×2⁻¹.

FIGS. 13A-B show miR34a copy number in tumors treated with FolamiR. MiR-34a levels measured by qRT-PCR from (FIG. 13A) breast cancer xenografted tumors (FIG. 13B) and lung adenocarcinoma KrasLSL-G12D/+;p53flx/flx tumors at 24 hours post last injection (n=5; error bars represent mean±s.d, statistical analysis performed with one-way ANOVA or Student's t-test).

FIGS. 14A-B show serum cytokines and Maximum Tolerated Dose Study. FIG. 14A shows serum obtained from FolamiR treated Nu/Nu mice bearing MDA-MB-231 tumors was evaluated for relevant cytokines: tumor necrosis factor (TNF)α, and interleukin (IL)-6 (n=5). Serum from lipopolysaccharides (LPS) treated mice was included as a positive detection control (n=2; statistical analysis was performed with a one-way ANOVA with post hoc Bonferroni correction). FIG. 14B shows body weight before and after intravenous administration of increasing doses of FolamiR-34a. Statistical analysis was performed with a two-way ANOVA with post hoc Bonferroni correction.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 20%, within 10%, within 5%, or within 1% of a stated value or of a stated limit of a range.

In the present disclosure the term “substantially” can allow for a degree of variability in a value or range, for example, within 70%, within 80%, within 90%, within 95%, or within 99% of a stated value or of a stated limit of a range.

Several embodiments of the invention are described by the following enumerated clauses and any combination of these embodiments with the embodiments described in this Detailed Description section is contemplated.

1. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and/or a therapeutic agent (TA) comprising an siRNA, an iRNA, or a microRNA;

wherein (L) optionally comprises at least one releasable linker; (B) is covalently linked to (L); and each of (A) and/or (TA) is covalently linked to (L).

1a. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and

a therapeutic agent comprising an siRNA, an iRNA, or a microRNA;

wherein (L) comprises at least one releasable linker; (B) is covalently linked to (L); and each (A) is covalently linked to (L).

2. The conjugate of clause 1 or 1a, or a pharmaceutically acceptable salt thereof, wherein (L) comprises at least one releasable linker.

3. The conjugate of clause 1 or 1a, or a pharmaceutically acceptable salt thereof, wherein the therapeutic agent (TA) is covalently linked to (L).

4. The conjugate of clause 1 or 1a, or a pharmaceutically acceptable salt thereof, wherein the therapeutic agent (TA) comprises an siRNA.

5. The conjugate of clause 1 or 1a, or a pharmaceutically acceptable salt thereof, wherein the therapeutic agent (TA) comprises an iRNA.

6. The conjugate of clause 1 or 1a, or a pharmaceutically acceptable salt thereof, wherein the therapeutic agent (TA) comprises a microRNA.

7. The conjugate of clause 1 or 1a, or a pharmaceutically acceptable salt thereof, wherein (B) is a folate.

8. The conjugate of clause 1 or 1a, or a pharmaceutically acceptable salt thereof, wherein (B) is a PSMA binding ligand.

9. The conjugate of clause 1 or 1a, or a pharmaceutically acceptable salt thereof, wherein (A) is an inhibitor of the Na⁺/H⁺ exchanger.

10. The conjugate of clause 1 or 1a, or a pharmaceutically acceptable salt thereof, wherein the ionophore (A) comprises nigericin or salinomycin.

11. The conjugate of clause 1 or 1a, or a pharmaceutically acceptable salt thereof, wherein (L) comprises a chain of about 7 to about 45 atoms.

12. The conjugate of clause 1 or 1a, having a formula selected from the group consisting of

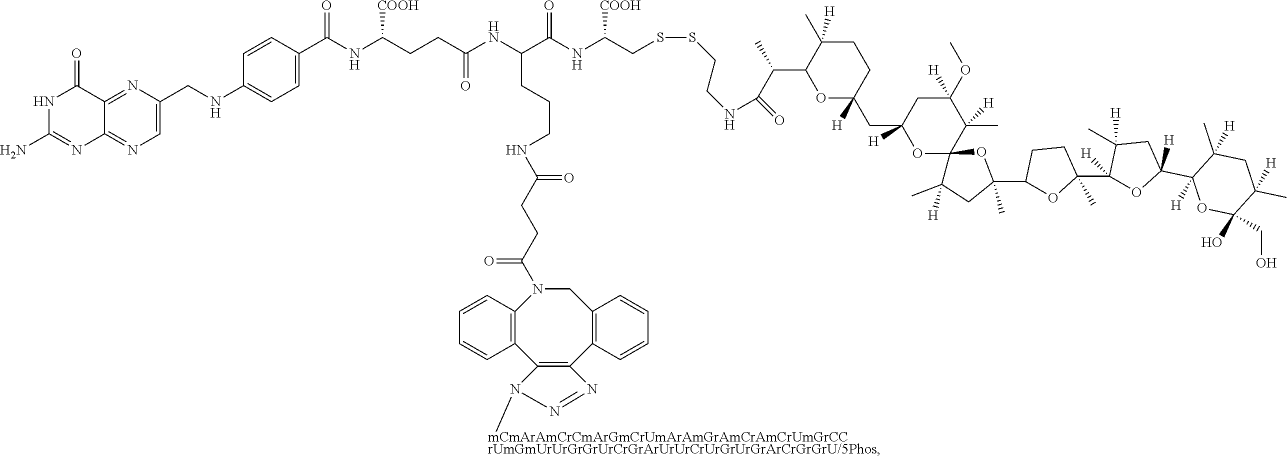

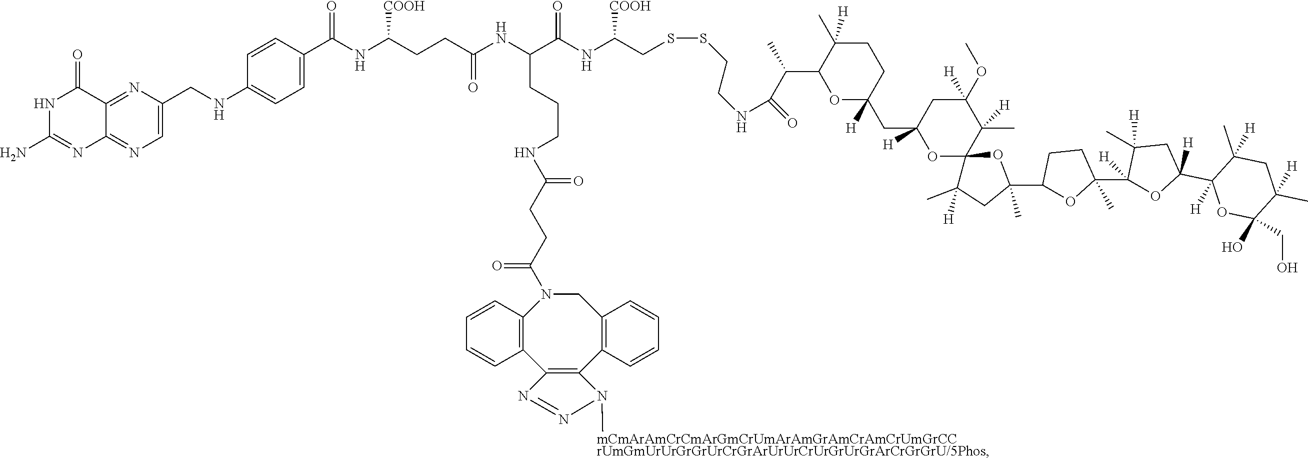

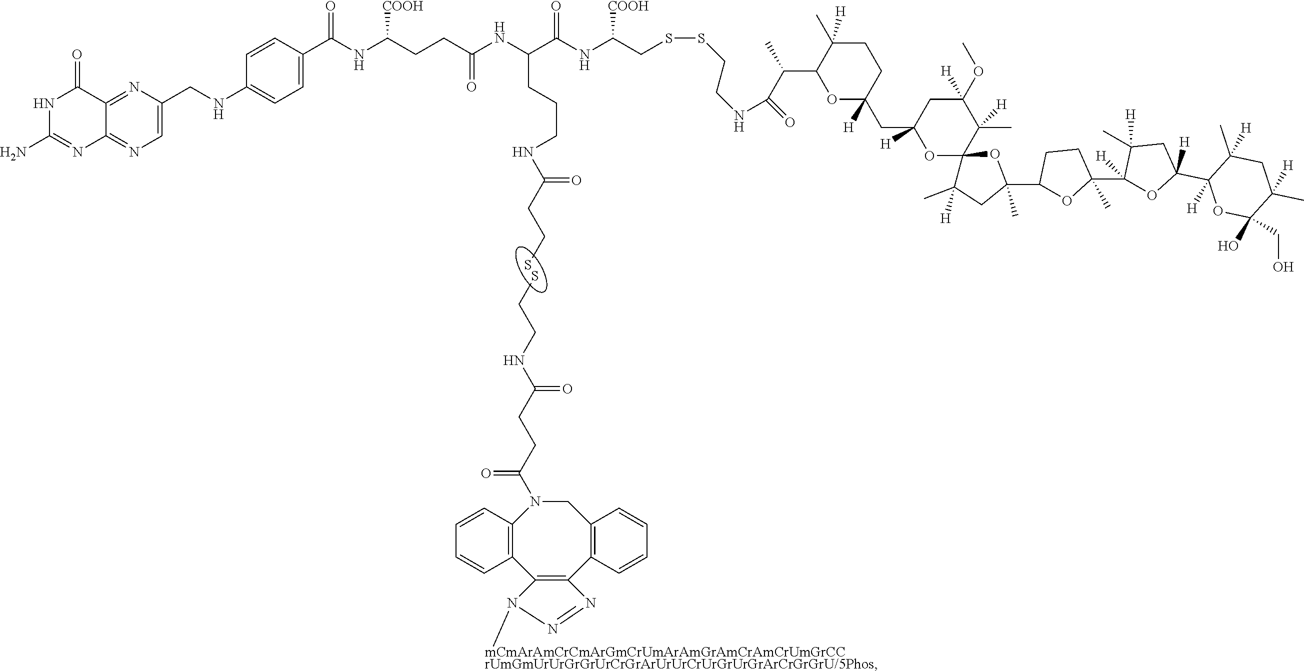

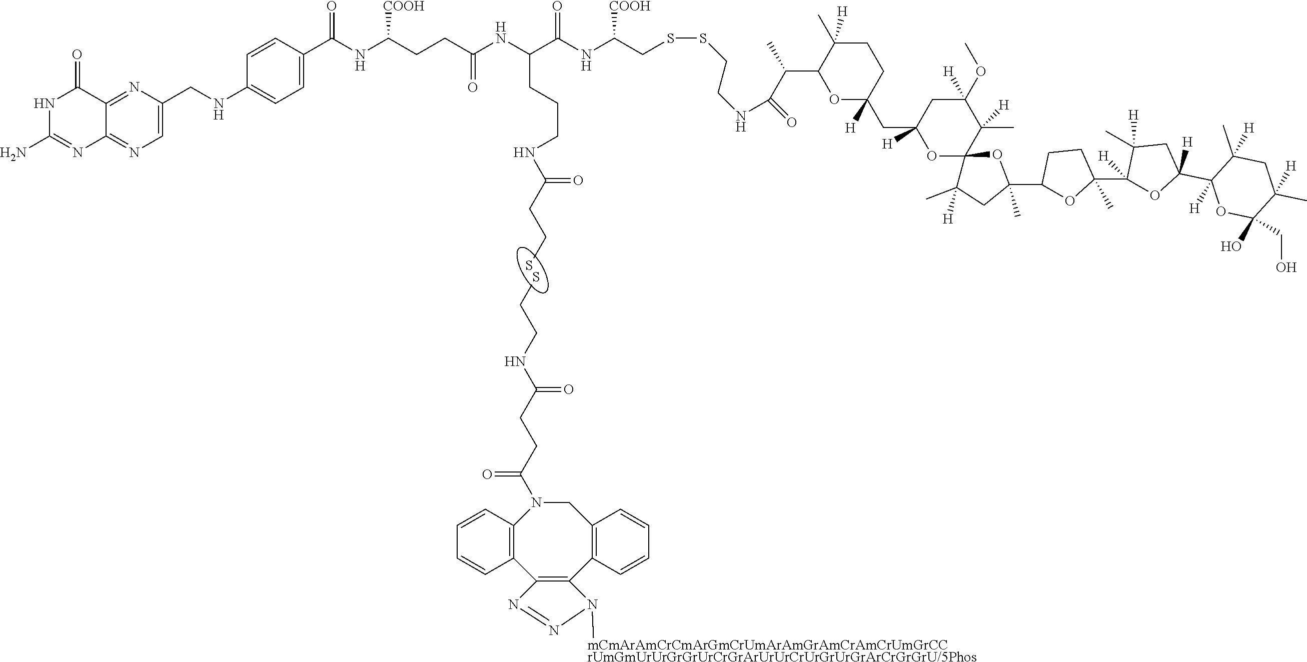

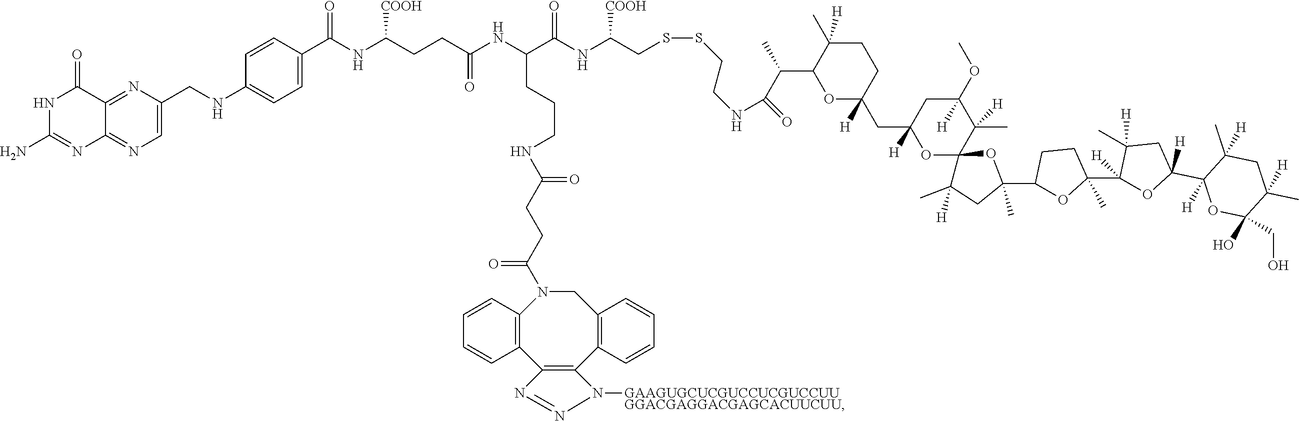

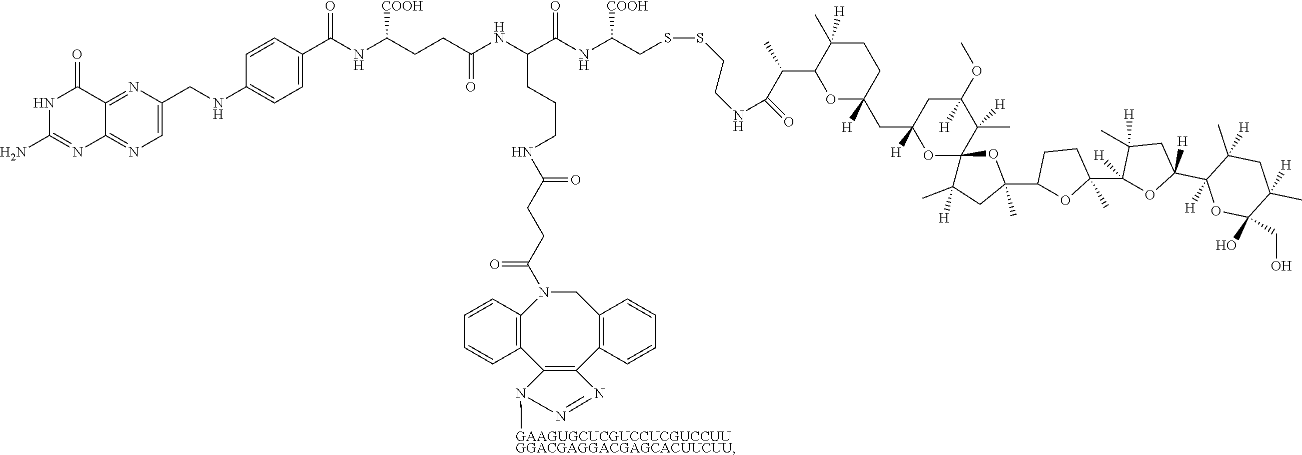

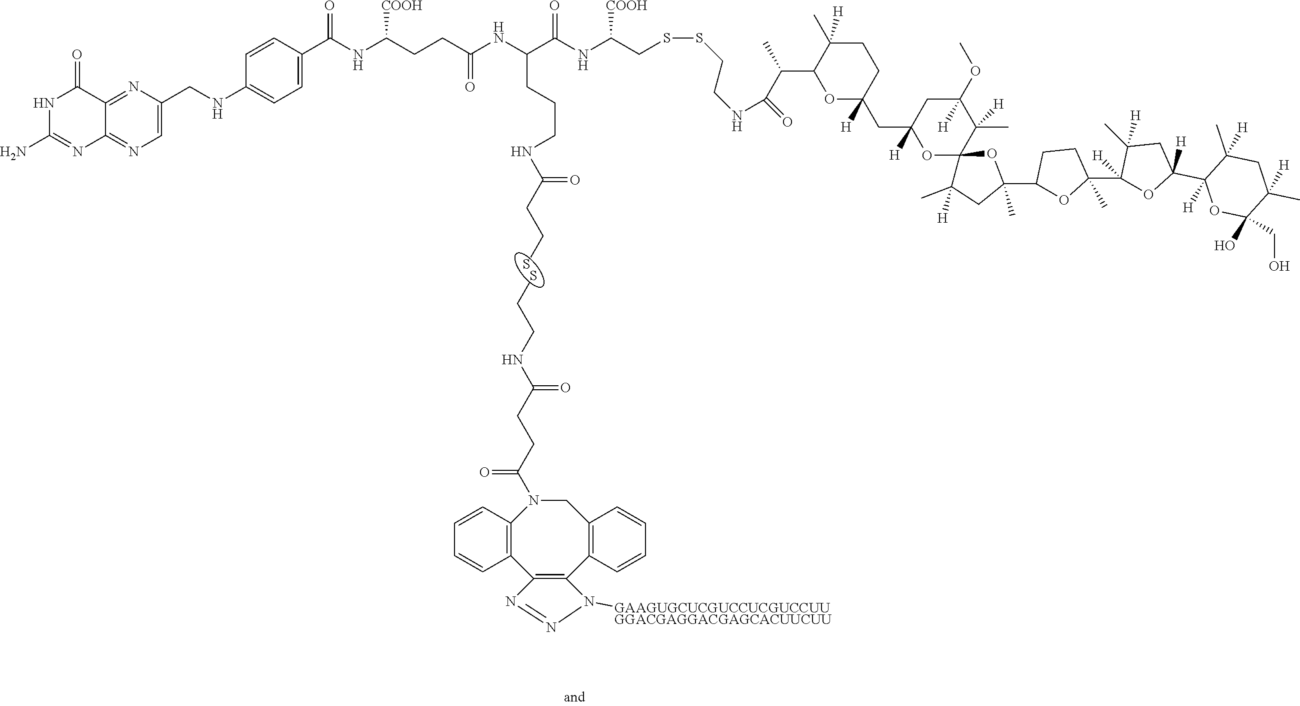

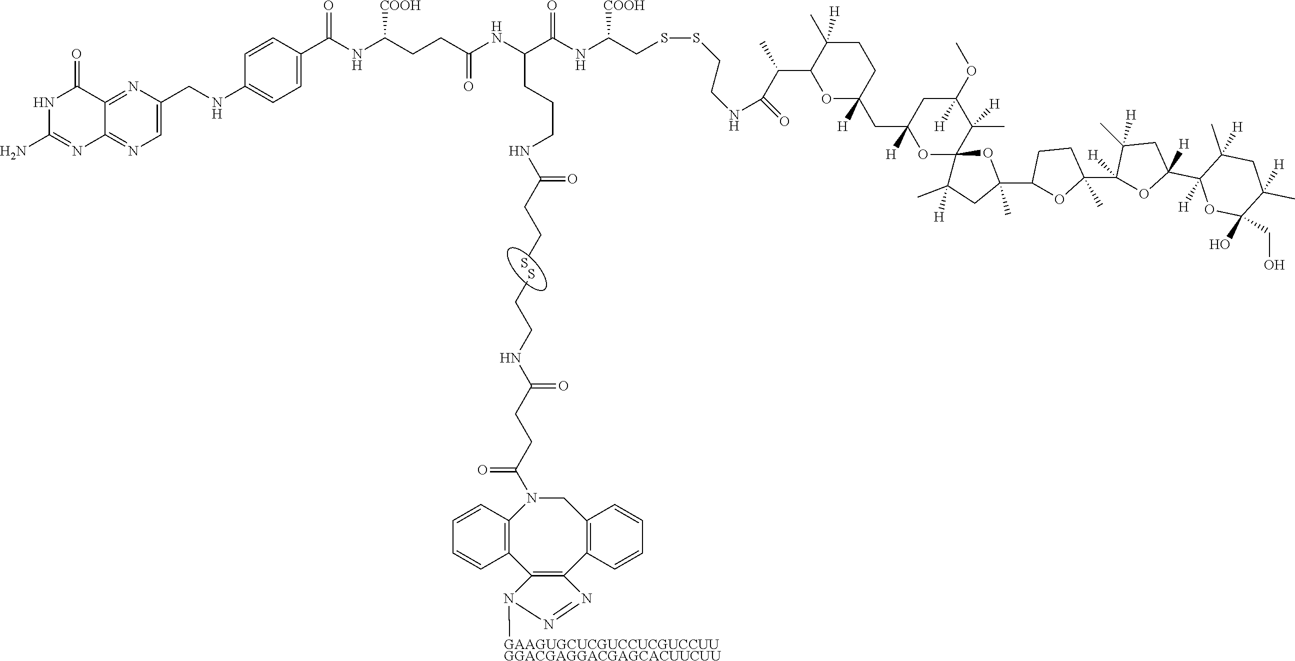

or a pharmaceutically acceptable salt thereof.

13. The conjugate of clause 1 having a formula

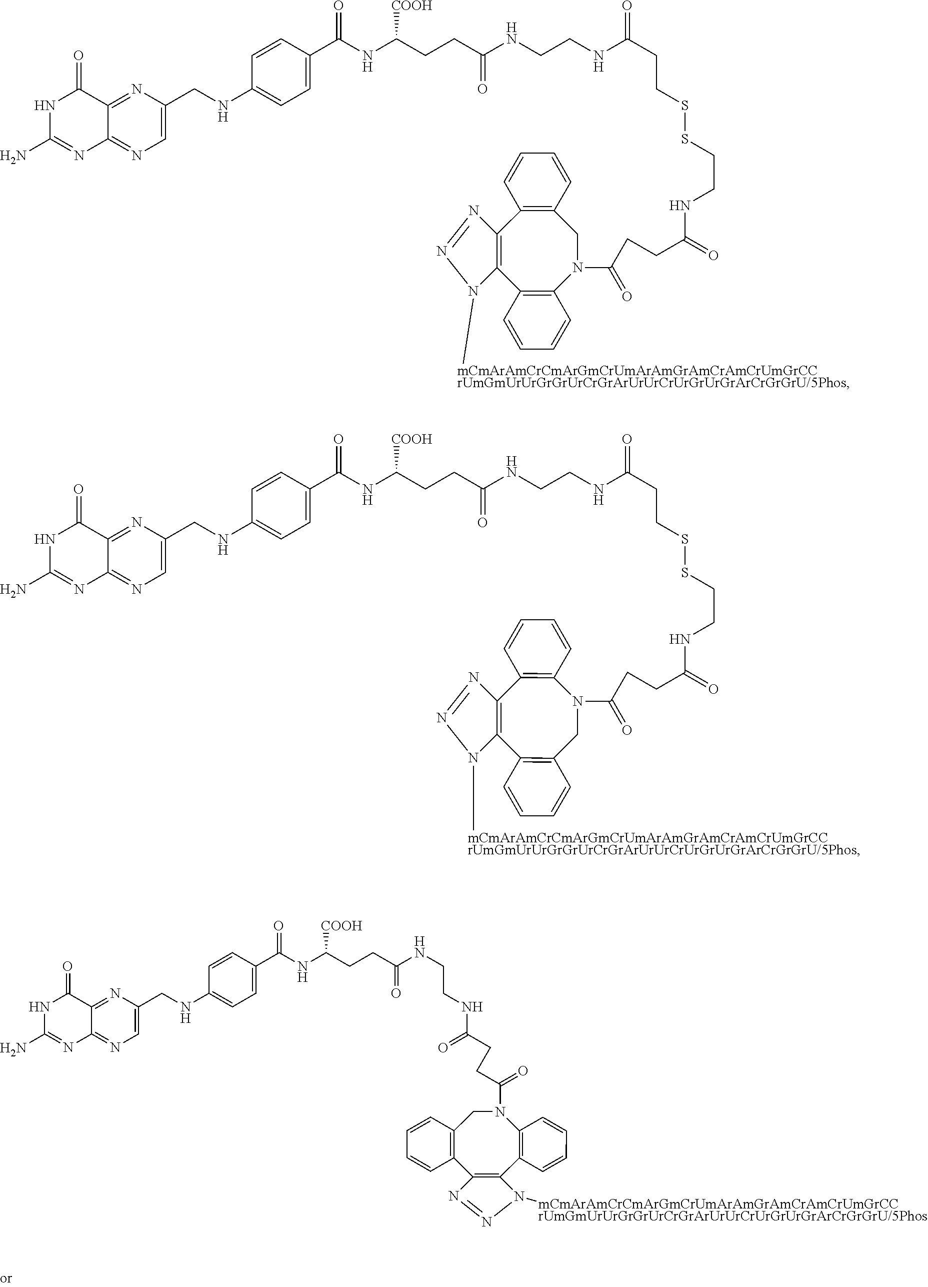

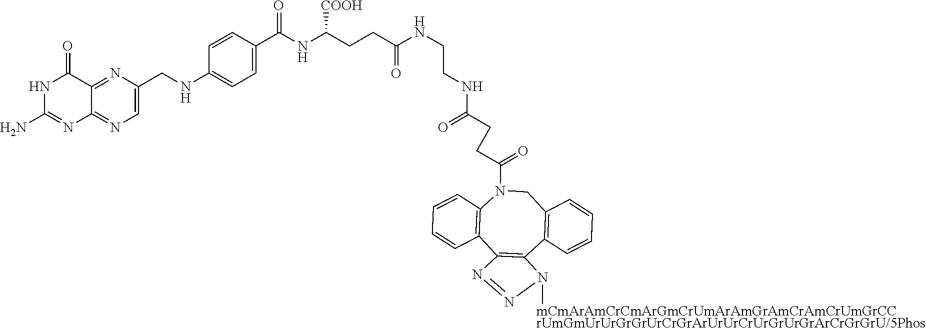

or a pharmaceutically acceptable salt thereof.

14. A pharmaceutical composition comprising at least one conjugate of any one of clauses 1 to 13, or a pharmaceutically acceptable salt thereof, and at least one pharmaceutically acceptable carrier or excipient.

15. A pharmaceutical composition comprising at least one conjugate of any one of clauses 1 to 13, or a pharmaceutically acceptable salt thereof, and an additional therapeutic agent.

16. A method of increasing the endosomal accumulation and escape of a therapeutic agent or an imaging agent, the method comprising the step of administering with the therapeutic agent or the imaging agent an effective amount of the conjugate of any one of clauses 1 to 13, or a pharmaceutically acceptable salt thereof.

17. The method of clause 16 wherein the therapeutic agent or the imaging agent is targeted to a cancer.

18. The method of clause 17 wherein the cancer is selected from the group consisting of ovarian, lung, breast, endometrial, brain, kidney, prostate, and colon cancer.

19. The method of clause 16 wherein the therapeutic agent is targeted to a site of inflammation.

20. The method of clause 19 wherein the site of inflammation is caused by an inflammatory disease selected from the group consisting of rheumatoid arthritis, osteoarthritis, atherosclerosis, diabetes, graft-versus-host disease, multiple sclerosis, osteomyelitis, psoriasis, Crohn's disease, Sjögren's syndrome, lupus erythematosus, and ulcerative colitis.

21. A conjugate, or a pharmaceutically acceptable salt thereof, comprising:

a ligand (B) targeted to a cell-surface receptor;

one or more linkers (L);

one or more of an ionophore (A) which couples efflux of protons (H⁺ ions) to influx of potassium ions (K⁺ ions); an RNA selected from an siRNA, an iRNA, and a microRNA; or an imaging agent (IA);

wherein (L) comprises at least one releasable linker; (B) is covalently linked to (L); and each of (A), the RNA and/or (IA) is covalently linked to (L).

22. A conjugate, or a pharmaceutically acceptable salt thereof, comprising:

a ligand (B) targeted to a cell-surface receptor;

one or more linkers (L);

one or more ionophores (A) each of which couples efflux of protons (H⁺ ions) to influx of potassium ions (K⁺ ions); and

a fluorescent dye comprising Cy5;

wherein (L) comprises at least one releasable linker; (B) is covalently linked to (L); and each (A) is covalently linked to (L).

23. The conjugate of clause 21 or 22, having a formula

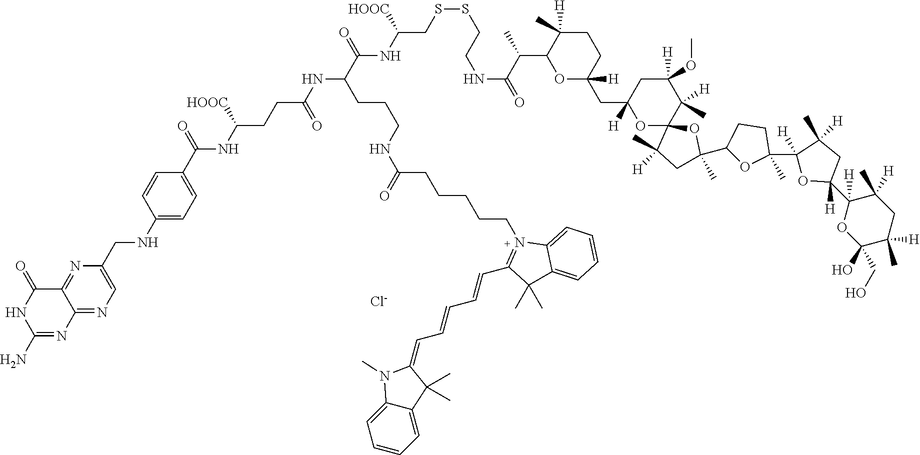

or a pharmaceutically acceptable salt thereof.

23. The conjugate of clause 21, having a formula

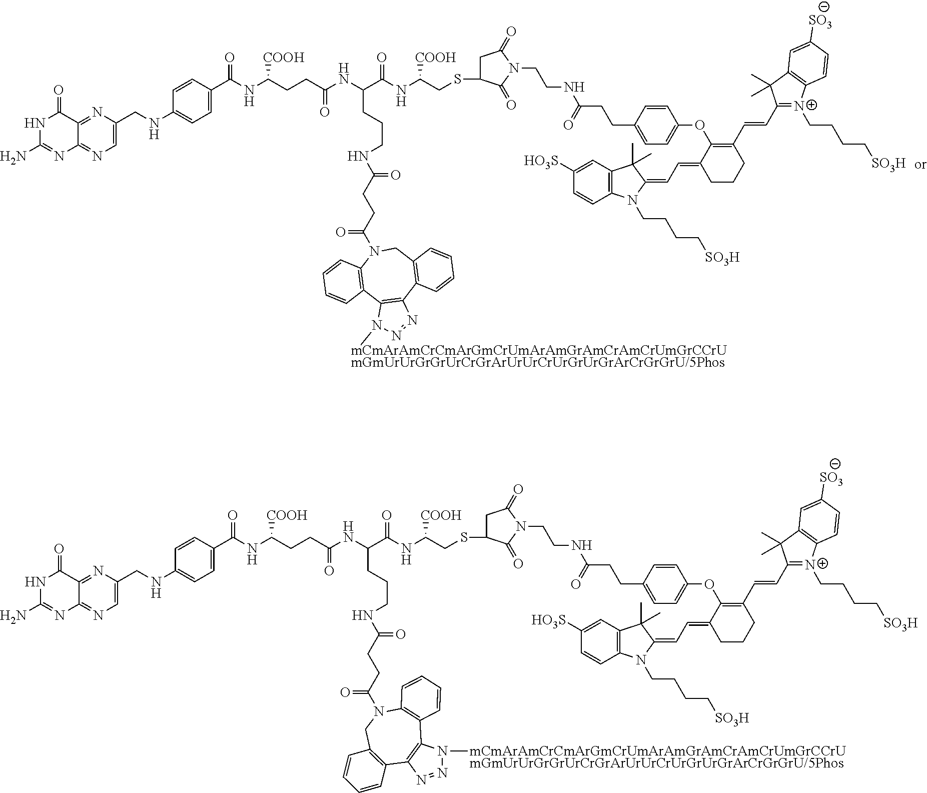

or a pharmaceutically acceptable salt thereof.

Several alternative embodiments of the invention are described by the following enumerated clauses and any combination of these embodiments with the embodiments described in this Detailed Description section is contemplated. It will be appreciated that each of the following embodiments can be combined with any other embodiment(s) described in the application to the extent that such embodiment(s) do not conflict with one another.

1. A conjugate comprising:

a ligand (B) targeted to a cell-surface receptor;

a linker (L); and

one or more ionophores (A) each of which couples efflux of protons (H⁺ ions) to influx of potassium ions (K⁺ ions);

wherein (L) comprises at least one releasable linker; (B) is covalently linked to (L); and each (A) is covalently linked to (L).

2. The conjugate of clause 1 wherein (L) comprises at least one releasable linker.

3. The conjugate of clause 1 or 2 further comprising a therapeutic agent, and/or an imaging agent wherein the therapeutic agent or the imaging agent is covalently linked to (L).

4. The conjugate of any of clauses 1 to 3 wherein (B) is targeted to a folate receptor or a prostate specific membrane antigen (PSMA).

5. The conjugate of clause 2 wherein (B) is a folate.

6. The conjugate of clause 5 further comprising a therapeutic agent.

7. The conjugate of clause 5 or 6 wherein (B) is folate.

8. The conjugate of clause 5 having the formula

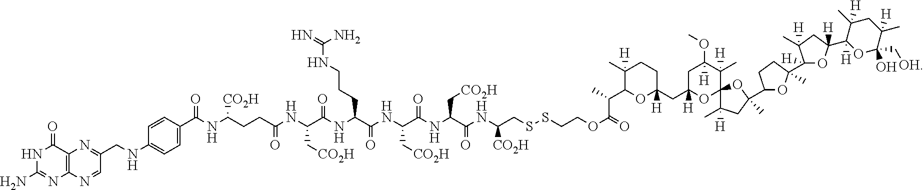

9. The conjugate of clause 5 having the formula

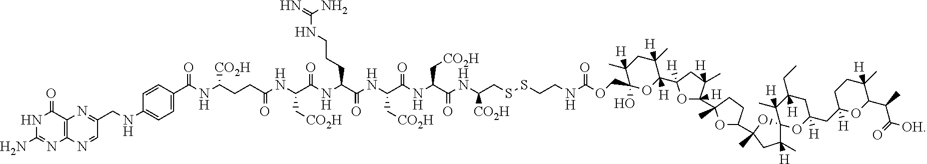

10. The conjugate of clause 6 having the formula

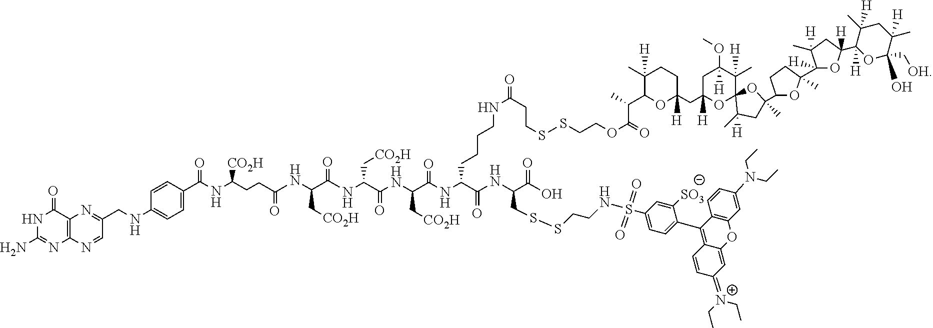

11. The conjugate of any one of clauses 1 to 4 wherein (B) is a PSMA binding ligand;

12. The conjugate of clause 11 further comprising a therapeutic agent or an imaging agent.

13. The conjugate of clause 11 or 12 wherein the PSMA binding ligand is 2-[3-(1-carboxy-2-mercaptoethyl)ureido]pentanedioic acid (MUPA) or 2-[3-(1,3-dicarboxypropyl)-ureido]pentanedioic acid (DUPA).

14. The conjugate of clause 13 having the formula

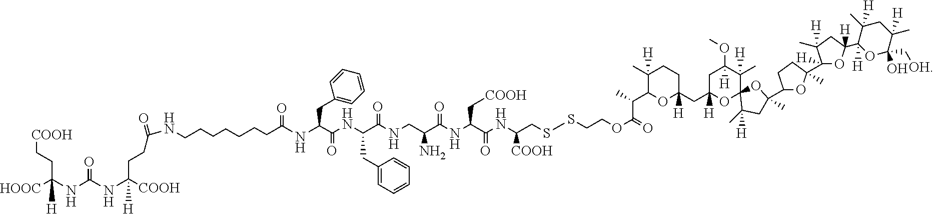

15. The conjugate of any of the preceding clauses 3-4, 6-7 or 12-13 wherein the therapeutic agent comprises a low molecular weight drug, a polypeptide, a peptide, an oligonucleotide, a nucleotide, an siRNA, an iRNA, a microRNA, a ribozyme, an antisense oligonucleotide, a protein, a glycoprotein, an antibody, an antigen, a synthetic amino acid, an aptamer, an oligosaccaride, or a polysaccaride.

16. The conjugate of clause 15 wherein the therapeutic agent is siRNA, miRNA or iRNA.

17. The conjugate of clause 15 wherein the therapeutic agent comprises a low molecular weight drug.

18. The conjugate of clause 15 wherein the therapeutic agent comprises a peptide or a synthetic amino acid.

19. The conjugate of clause 15 wherein the therapeutic agent comprises a low molecular weight chemotherapeutic agent.

20. The conjugate of clause 19 wherein the therapeutic agent comprises a taxane or an analog thereof, a vinca alkaloid or an analog thereof, camptothecin or an analog thereof, a tubulysin or an analog thereof, or doxorubicin or an analog thereof.

21. The conjugate of clause 15 wherein the therapeutic agent comprises a low molecular weight anti-inflammatory agent.

22. The conjugate of clause 15 wherein the therapeutic agent comprises a lipophilic anti-inflammatory steroid.

23. The conjugate of clause 3 or 12 comprising an imaging agent.

24. The conjugate of clause 23 wherein the imaging agent comprises a fluorescent dye.

25. A conjugate of any of the preceding clauses wherein (A) is an inhibitor of the Na⁺/H⁺ exchanger.

26. The conjugate of clause 25 further comprising an ionophore wherein the ionophore couples efflux of protons (H⁺ ions) to influx of potassium ions (K⁺ ions).

27. The conjugate of clause 25 wherein the inhibitor is amiloride or HOE 694.

28. The conjugate of any of clauses 25-27 wherein the inhibitor is amiloride.

29. The conjugate of any of the preceding clauses 1-7, 11-13, 15-24 and 26-28 wherein the ionophore (A) is selected from the group consisting of nigericin or salinomycin.

30. The conjugate of clause 29 wherein the ionophore is nigericin.

31. The conjugate of any of clauses 1-7, 11-13 and 15-30 wherein (L) comprises a chain of about 7 to about 45 atoms.

32. A pharmaceutical composition comprising the conjugate of any of clauses 1-31, and 15-22 and further comprising at least one pharmaceutically acceptable carrier or excipient.

33. A pharmaceutical composition comprising the conjugate as described in any of clauses 3, 12 and 15-22 further comprising an additional therapeutic agent.

34. A method of increasing the endosomal accumulation and escape of a therapeutic agent, or an imaging agent comprising the step of administering with the therapeutic agent or the imaging agent an effective amount of a ligand-ionophore conjugate wherein the ionophore couples efflux of protons (H⁺ ions) to influx of potassium ions (K⁺ ions) and wherein the therapeutic agent or the imaging agent is targeted to a cell-surface receptor.

35. The method of clause 34 wherein the ionophore is selected from the group consisting of nigericin or salinomycin.

36. The method of clause 35 wherein the ionophore is nigericin.

37. The method of any of clauses 34-36 wherein the imaging agent or the therapeutic agent is not linked to the conjugate.

38. The method of any of clauses 34-36 wherein the imaging agent or the therapeutic agent is linked to the conjugate.

39. The method of clause 37 or 38 wherein the imaging agent or the therapeutic agent is targeted to the same receptor as the ligand-ionophore conjugate.

40. The method of clause 37 or 38 wherein the ligand-ionophore conjugate is the conjugate of any of clauses 1-2, 4 and 29-31.

41. The method of clause 39 wherein the ligand-ionophore conjugate is a conjugate of formula (B)-(L)-(A) and further comprises the imaging agent or the therapeutic agent, covalently linked to (L) and wherein the therapeutic agent or the imaging agent is as described in any of clauses 3 or 15-24.

42. The method of any of clauses 34-41 wherein the cell-surface receptor targeted by the ligand-ionophore conjugate is the folate receptor or the prostate specific membrane antigen (PSMA).

43. The method of clause 42 wherein the cell-surface receptor targeted by the ligand-ionophore conjugate is the folate receptor.

44. The method of clause 42 wherein the cell-surface receptor targeted by the ligand-ionophore conjugate is PSMA.

45. The method of clause 43 or 44 wherein the therapeutic agent or the imaging agent is targeted to a cancer or a site of inflammation.

46. The method of clause 45 wherein the cancer is selected from the group consisting of ovarian, lung, breast, prostate, endometrial, brain, kidney and colon cancer.

47. The method of clause 46 wherein the cancer is lung cancer.

48. The method of clause 46 wherein the cancer is ovarian cancer.

49. The method of clause 45 wherein the therapeutic agent or imaging agent is targeted to a site of inflammatory disease.

50. The method of clause 49 wherein the inflammatory disease is selected from the group consisting of rheumatoid arthritis, osteoarthritis, atherosclerosis, diabetes, graft-versus-host disease, multiple sclerosis, osteomyelitis, psoriasis, Sjögren's syndrome, lupus erythematosus, Crohn's disease, and ulcerative colitis.

51. The method of clause 42 wherein the cell-surface receptor targeted by the ligand-ionophore conjugate is the prostate specific membrane antigen (PSMA).

52. The method of clause 51 wherein the ligand-ionophore conjugate is the conjugate described in any of clauses 11-24 and 29-31.

53. The method of clause 51 or 52 wherein the targeted cell-surface receptor is over-expressed PSMA.

54. The method of clause 53 wherein the therapeutic agent or the imaging agent is targeted to a malignant prostate cell population.

55. The method of any of clauses 34-54 comprising the administration of an inhibitor of the Na⁺/H⁺ exchanger (antiporter).

56. The method of clause 55 wherein the inhibitor of the Na⁺/H⁺ exchanger (antiporter) is amiloride or HOE 694.

57. The method of clause 55 or 56 wherein the inhibitor of the Na⁺/H⁺ exchanger (antiporter) is conjugated to the ligand.

58. The method of clause 55 or 56 wherein the inhibitor of the Na⁺/H⁺ exchanger (antiporter) is covalently linked to the ligand-ionophore conjugate and is releasable.

59. The method of any of clauses 34-58 wherein the imaging agent or the therapeutic agent is administered as a liposome, dendrimer or large molecular weight polymer complex in a targeted form.

60. The method of any of clauses 34-59 wherein the imaging agent or the therapeutic agent comprises an anticancer agent, an anti-inflammatory agent, a radionuclide, or a fluorescent dye.

61. The method of clause 60 wherein the therapeutic agent comprises a vinca alkaloid, doxorubicin, an antifolate or a corticosteroid.

62. Use of a folate-targeted ligand-ionophore conjugate as described in any of clauses 5-10, 15-20, 23-24 and 29-31 for the imaging or treatment of a cancer that expresses or overexpresses the folate receptor.

63. Use of a folate-targeted ligand-ionophore conjugate as described in any of clauses 5-10, 15-20, 23-24 and 29-31 for the manufacture of an agent for use in a method for imaging or treatment of a cancer that expresses or overexpresses the folate receptor.

64. An agent for use in imaging or treatment of a cancer that expresses or overexpresses the folate receptor, comprising a folate-targeted ligand-ionophore conjugate as described in any of clauses 5-10, 15-20, 23-24 and 29-31.

65. A method of using an effective amount of a folate-targeted ligand-ionophore conjugate as described in any of clauses 5-10, 15-20, 23-24 and 29-31 in a method for imaging or treatment of a cancer, that expresses or overexpresses the folate receptor, in a subject in need thereof.

66. Use of a folate-targeted ligand-ionophore conjugate as described in any of clauses 5-10, 15-18, 21-24 and 29-31 for imaging or treatment of an inflammatory disease at a site of inflammation.

67. Use of a folate-targeted ligand-ionophore conjugate as described in any of clauses 5-10, 15-18, 21-24 and 29-31 for the manufacture of an agent for use in a method for imaging or treatment of an inflammatory disease at a site of inflammation.

68. An agent for use in imaging or treatment of an inflammatory disease, comprising a folate-targeted ligand-ionophore conjugate as described in any of clauses 5-10, 15-18, 21-24 and 29-31.

69. A method of using an effective amount of a folate-targeted ligand-ionophore conjugate as described in any of clauses 5-10, 15-18, 21-24 and 29-31 for imaging or treatment of an inflammatory disease in a subject in need thereof.

70. Use of a PSMA-targeting ligand-ionophore conjugate as described in any of clauses 11-14, 15-20, 23-24 and 29-31 for the imaging or treatment of a cancer that expresses or overexpresses PSMA.

71. Use of a PSMA-targeting ligand-ionophore conjugate as described in any of clauses 11-14, 15-20, 23-24 and 29-31 for the manufacture of an agent for use in a method for imaging or treatment of a cancer that expresses or overexpresses PSMA.

72. An agent for use in imaging or treatment of a cancer that expresses or overexpresses PSMA, comprising a folate-targeted ligand-ionophore conjugate as described in any of clauses 11-14, 15-20, 23-24 and 29-31.

73. A method of using an effective amount of a folate-targeted ligand-ionophore conjugate as described in any of clauses 12-13, 15-20, 23-24 and 29-31 in a method for imaging or treatment of a cancer, that expresses or overexpresses PSMA, in a subject in need thereof.

74. Use of a folate-targeted ligand-ionophore conjugate as described in any of clauses 5-10, 15-18, 21-24 and 29-31 in association with a therapeutic agent or an imaging agent wherein the conjugate is internalized by endocytosis.

75. Use of a folate-targeted ligand-ionophore conjugate as described in any of clauses 5-10, 15-18, 21-24 and 29-31 for the manufacture of an agent for use in a method for imaging or treatment of a cancer, for use in association with a therapeutic agent, or an imaging agent that is internalized by endocytosis.

76. An agent for use in imaging or treatment of a cancer in association with a therapeutic agent, or an imaging agent that is internalized by endocytosis, wherein the agent comprises a folate-targeted ligand-ionophore conjugate as described in any of clauses 5-10, 15-18, 21-24 and 29-31.

77. A method of using an effective amount of a folate-targeted ligand-ionophore conjugate as described in any of clauses 5-10, 15-18, 21-24 and 29-31 in a method for imaging or treatment of a cancer in association with a therapeutic agent, or an imaging agent that is internalized by endocytosis.

78. Use of a folate-targeted ligand-ionophore conjugate as described in any of clauses 5-10, 15-18, 21-24 and 29-31 in association with a therapeutic agent, or an imaging agent that is internalized by endocytosis for imaging or treating an inflammatory disease at a site of inflammation.

79. Use of a folate-targeted ligand-ionophore conjugate as described in any of clauses 5-10, 15-18, 21-24 and 29-31 for the manufacture of an agent for use in a method for imaging or treatment of an inflammatory disease in association with a therapeutic agent, or an imaging agent that is internalized by endocytosis.

80. An agent for use in imaging or treatment of an inflammatory disease in association with a therapeutic agent, or an imaging agent that is internalized by endocytosis, wherein the agent comprises a folate-targeted ligand-ionophore conjugate as described in any of clauses 5-10, 15-18, 21-24 and 29-31.

81. A method of using an effective amount of a folate-targeted ligand-ionophore conjugate as described in any of clauses 5-10, 15-18, 21-24 and 29-31 in a method for imaging or treatment of an inflammatory disease in association with a therapeutic agent, or an imaging agent that is internalized by endocytosis.

82. Use of a PSMA-targeted ligand-ionophore conjugate as described in any of clauses 11-14, 15-20, 23-24 and 29-31 in association with a therapeutic agent, or an imaging agent that is internalized by endocytosis, for the imaging or treatment of a cancer that expresses or overexpresses PSMA.

83. Use of a PSMA-targeted ligand-ionophore conjugate as described in any of clauses 11-14, 15-20, 23-24 and 29-31 for the manufacture of an agent for use in a method for imaging or treatment, in association with a therapeutic agent, or an imaging agent that is internalized by endocytosis, of a cancer which expresses or overexpresses PSMA.

84. An agent for use in imaging or treatment of a cancer, in association with a therapeutic agent, or an imaging agent that is internalized by endocytosis, wherein the agent comprises a PSMA-targeting ligand-ionophore conjugate as described in any of clauses 11-14, 15-20, 23-24 and 29-31.

85. A method of using an effective amount of a PSMA-targeting ligand-ionophore conjugate as described in any of clauses 11-14, 15-20, 23-24 and 29-31 in a method for imaging or treatment of a cancer that expresses or overexpresses PSMA, in association with a therapeutic agent, or an imaging agent that is internalized by endocytosis.

As used herein the term “nucleotide” is given its usual and customary meaning, and can include ribonucleotides. The abbreviations for ribonucleotides (e.g. A, G, C, U) are given their usual and customary meaning. In some embodiments, conjugates provided herein can comprise an RNA sequence (i.e. a micro RNA or “miRNA”). In some embodiments, ribonucleotides are represented by their customary one letter abbreviation immediately preceded by the letter “r” in the sequence (e.g. rA, rG, rC). In some embodiments, the RNA sequence can include modified ribonucleotides. In such embodiments, the ribonucleotides are represented by a one letter abbreviation immediately preceded in the sequence by a letter to denote the modification. It will be appreciated that common modifications include, but are not limited to, methyl (m), ethyl (e), amino (a), deamino (o), and the like. For example, a methylated cytidine can be denoted by mC in a sequence as described herein. It will be appreciated that other modifications known in the art are also contemplated by the present disclosure.

As used herein, the term “conjugate” means the ligand-ionophore (ligand-ionophore means with or without a linker between the ligand and the ionophore) conjugate or a ligand-ionophore (ligand-ionophore means with or without a linker between the ligand and the ionophore) conjugate with a linked therapeutic agent or imaging agent, or a pharmaceutically acceptable salt of the conjugate, or a solvate thereof; and the conjugate may be present in solution or suspension in an ionized form, including a protonated form.

As used herein, the term “ionophore” also means a cluster of ionophores, for example, in a dendritic construct. Similarly, a therapeutic agent, or an imaging agent conjugated to the ligand-ionophore conjugate may be a cluster of agents, for example, in a dendritic construct.

As used herein, the term “releasable” means that the particular moiety is covalently linked to the linker (L) by a releasable linker.

As used herein, the terms drug, therapeutic agent, chemotherapeutic agent, etc. include analogs thereof which can be incorporated into a conjugate or administered separately, in targeted form.

As used herein the term “endocytosis” has its art-recognized meaning and includes several analogous processes, such as the process of PSMA internalization.

It will be appreciated that the therapeutic agent or the imaging agent may comprise an agent prepared by synthetic chemistry, an agent isolated from a natural source, a biologically synthesized agent, or a macromolecular structure such as a liposome or a dendrimer comprising the therapeutic agent, or the imaging agent.

The therapeutic agent can be any molecule capable of modulating or otherwise modifying cell function, including pharmaceutically active compounds. Therapeutic agents may be antibiotics; analgesics; bronchodilators; beta-blockers; antimicrobial agents; antihypertensive agents; cardiovascular agents including antiarrhythmics, cardiac glycosides, antianginals and vasodilators; central nervous system agents including stimulants, psychotropics, antimanics and antidepressants; antiviral agents; antihistamines; cancer drugs including chemotherapeutic agents; tranquilizers; anti-depressants; H-2 antagonists; anticonvulsants; antinauseants; prostaglandins and prostaglandin analogs; muscle relaxants; anti-inflammatory substances; stimulants; decongestants; antiemetics; diuretics; antispasmodics; antiasthmatics; anti-Parkinson agents; expectorants; cough suppressants; mucolytics; and mineral and nutritional additives, or any other therapeutic agent known to a skilled artisan.

When a therapeutic agent is an anticancer agent, the therapeutic agent can be any drug known in the art which is cytotoxic, enhances tumor permeability, inhibits tumor cell proliferation, promotes apoptosis, decreases anti-apoptotic activity in tumor cells, enhances an endogenous immune response directed to the tumor cells, or is useful for treating a cancer.

Therapeutic agents suitable for use in accordance with this invention include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and antifolates, such as methotrexate and aminopterin, busulfan, carboplatin, chlorambucil, cisplatin and other platinum compounds, taxanes, such as tamoxiphen, taxol, paclitaxel, paclitaxel derivatives, Taxotere™, and the like, maytansines and analogs and derivatives thereof, cyclophosphamide, daunomycin, doxorubicin, rhizoxin, T2 toxin, plant alkaloids, prednisone, hydroxyurea, teniposide, mitomycins, discodermolides, microtubule inhibitors, epothilones, everolimus, tubulysin, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vincristine, vinblastine, and analogs and derivatives thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, doxorubicin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin.

When the therapeutic agent is a chemotherapeutic agent, it is selected from those which are, for example, cytotoxic themselves or can work to enhance tumor permeability, and are also suitable for use in the method of the invention in combination with the ligand-ionophore conjugates. Such chemotherapeutic agents include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, aminopterin, any art-recognized antifolate, an everolimus, busulfan, carboplatin, chlorambucil, cisplatin and other platinum compounds, tamoxiphen, taxol, paclitaxel, paclitaxel derivatives, Taxotere™, cyclophosphamide, daunomycin, doxorubicin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin.

When the therapeutic agent is an anti-inflammatory agent, it may comprise an anti-inflammatory steroid, a topically administered anti-inflammatory steroid, a water soluble anti-inflammatory steroid, a non-steroidal anti-inflammatory drug (NSAID), which also may be denoted as a non-steroidal anti-inflammatory agent (NSAIA) or as a non-steroidal anti-inflammatory medicine (NSAIM), or another drug useful in the treatment of rheumatoid arthritis or another autoimmune disease including an antiproliferative, immunomodulator or immunosuppressant agent.

When the therapeutic agent is an anti-inflammatory agent it may comprise a systemically administered (lipophilic) anti-inflammatory steroid. In one embodiment, the anti-inflammatory steroid is betamethasone, dexamethasone, flumethasone, methylprednisolone, paramethasone, prednisolone, prednisone, triamcinolone, hydrocortisone, or cortisone. In a further embodiment, the anti-inflammatory steroid is betamethasone.

When the therapeutic agent comprises a topically administered anti-inflammatory steroid, the anti-inflammatory steroid can be alcomethasone dipropionate, amcinonide, betamethasone dipropionate, betamethasone monopropionate, betamethasone 17-valerate, budesonide, budesonide disodium phosphate, ciclomethasone, clobetasol-17-propionate, clobetasone-17-butyrate, cortisone acetate, deprodone propionate, desonide, desoxymethasone, dexamethasone acetate, diflucortolone valerate, diflurasone diacetate, diflucortolone, difluprednate, flumetasone pivalate, flunisolide, fluocinolone acetonide acetate, fluocinonide, fluocortolone, fluocortolone caproate, fluocortolone hexanoate, fluocortolone pivalate, fluormetholone acetate, fluprednidene acetate, fluticasone propionate, halcinonide, halometasone, hydrocortisone acetate, hydrocortisone-17-butyrate, hydrocortisone-17-valerate, medrysone, methylprednisolone acetate, mometasone furoate, parametasone acetate, prednicarbate, prednisolone acetate, prednylidene, rimexolone, tixocortol pivalate, triamcinolone acetonide, triamcinolone alcohol or triamcinolone hexacetonide. In one embodiment, it is budesonide, flunisolide or fluticasone propionate.

When the therapeutic agent is an anti-inflammatory agent it may comprise a water soluble anti-inflammatory steroid. In one embodiment, the anti-inflammatory steroid can be betamethasone sodium phosphate, desonide sodium phosphate, dexamethasone sodium phosphate, hydrocortisone sodium phosphate, hydrocortisone sodium succinate, cortisone sodium phosphate, cortisone sodium succinate, methylprednisolone disodium phosphate, methylprednisolone sodium succinate, methylprednisone disodium phosphate, methylprednisone sodium succinate, prednisolone sodium phosphate, prednisolone sodium succinate, prednisone sodium phosphate, prednisone sodium succinate, prednisolamate hydrochloride, triamcinolone acetonide disodium phosphate or triamcinolone acetonide dipotassium phosphate. In one embodiment, the therapeutic agent is budesonide disodium phosphate.

When the therapeutic agent is an anti-inflammatory agent it can be a non-steroidal anti-inflammatory drug (NSAID), and the NSAID can comprise a propionic acid derivative such as, for example, ibuprofen, naproxen, fenoprofen, ketoprofen, flurbiprofen or oxaprozin; or the NSAID can comprise an acetic acid derivative, such as, for example, indomethacin, sulindac, etodolac or diclofenac; or the NSAID can comprise an oxicam derivative, such as, for example, piroxicam, meloxicam, tenoxicam, droxicam, lornoxicam or isoxicam; or the NSAID can comprise a fenamic acid derivative, such as, for example, mefenamic acid, meclofenamic acid, flufenamic acid or tolfenamic acid; or the NSAID can comprise a selective COX-2 (cyclooxygenase-2) inhibitor (coxib), such as, for example, celecoxib, rofecoxib, valdecoxib, parecoxib, lumiracoxib or etoricoxib.

When the therapeutic agent is an anti-inflammatory agent it can comprise a drug useful in the treatment of rheumatoid arthritis or another autoimmune disease including an antiproliferative, immunomodulator or immunosuppresant agent. In one embodiment the anti-inflammatory agent can comprise, for example, aspirin, methotrexate, sulfasalazine, D-penicillamine, nambumetone, aurothioglucose, auranofin, other gold-containing compound, colloidal gold, cyclosporin, tacrolimus, pimecrolimus or sirolimus.

In some embodiments, the therapeutic agent is a biologic, such as a polypeptide, a peptide, an oligonucleotide, a nucleotide, an siRNA, an iRNA, a microRNA, a ribozyme, an antisense oligonucleotide, a protein, a glycoprotein, an antibody, an antigen, a synthetic amino acid, an aptamer, an oligosaccaride, or a polysaccaride. In some embodiments, the therapeutic agent comprises an siRNA, an iRNA, or a microRNA.

When the agent is an imaging agent (IA), the agent may comprise a fluorescent agent, an X-ray contrast agent, such as for example iobitridol, a PET imaging agent, a near IR dye (NIR dye), or a radionuclide, such as for example, an isotope of gallium, indium, copper, technitium or rhenium. Fluorescent agents include fluorescein, 5-amino-fluorescein, 6-amino-fluorescein, fluorescein isocyanate (FITC), NHS-fluorescein, Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, BODIPY fluorescent agents, including but not limited to BODIPY F1, BODIPY 505, and the like, rhodamine fluorescent agents, including but not limited to 5-carboxytetramethylrhodamine (5-TAMRA), rhodamine B, rhodamine 6G, TRITC, Texas Red, rhodamine 123, sulforhodamine 101, tetramethylrhodamine, and the like, DyLight fluorescent agents, including but not limited to DyLight 647, DyLight 680, DyLight 800, and the like, CW 800, phycoerythrin, and others. Representative near infrared dyes that may be used in accordance with the present teachings include but are not limited to LS288, IR800, SP054, S0121, KODAK, IRD28, S2076, S0456, and derivatives thereof.

Certain isotopically-labelled conjugates, for example, those incorporating a radioactive isotope, may be useful in drug and/or substrate tissue distribution studies. The radioactive isotopes tritium (i.e., ³H), and carbon-14 (i.e., ¹⁴C) are particularly useful for this purpose in view of their ease of incorporation and ready means of detection.

Substitution with positron emitting isotopes, such as ¹¹C, ¹⁸F, and ¹³N, may be useful in Positron Emission Topography (PET) studies for examining substrate receptor occupancy. Isotopically-labeled conjugates may generally be prepared by conventional techniques known to those skilled in the art or by processes analogous to those described in the accompanying Examples using an appropriate isotopically-labeled reagents in place of the non-labeled reagent previously employed.

The preparation and use of releasable linkers for releasing the “payload” is well documented. The conjugation of the ligand and ionophore, may utilize procedures which are analogous to those used for single or dual conjugation of a drug employing releasable linkers, as described, for example, inter alia, in WO 2003/097647, WO 2004/069159, WO 2006/012527, WO 2007/022493, WO 2007/022494, WO 2009/002993 WO 2010/033733 and WO 2010/045584. The disclosures of each of the foregoing patent applications are incorporated herein by reference. These same references also describe methods that can be used to link the therapeutic agent or the imaging agent to the ligand-ionophore conjugate, or to prepare separate ligand-therapeutic agent or ligand-imaging agent compounds.

Uses and preparation of PMSA targeting ligands and intermediates linked to ionophores useful for the instant invention are described, inter alia, in WO 2009/026177, WO 2010/045598 and WO 2011/106639. The disclosures of each of the foregoing patent applications are incorporated herein by reference. These same references also describe methods that can be used to link the therapeutic agent or the imaging agent to the ligand-ionophore conjugate, or to prepare separate ligand-therapeutic agent or ligand-imaging agent compounds. DUPA binds selectively to prostate-specific membrane antigen (Ligand-Targeted Delivery of Small Interfering RNAs to Malignant Cells and Tissues. Thomas, M., Kularatne, S. A., Qi, L., Kleindl, P., Leamon, C. P., Hansen, M. J., and Low, P. S. Ann. N.Y. Acad. Sci. 1175, 32-39 (2009)).

In an illustrative example, nigericin, an ionophore and hydrogen ion/potassium ion antiporter, containing free hydroxyl and carboxylic acid functional groups is chemically attached to a ligand through releasable linkers bound to the hydroxyl or carboxylic acid groups, as shown in the examples. In one illustrative example, in a folate-nigericin ester conjugate, a folate ligand is conjugated via a disulfide containing linker to nigericin through the carboxylic acid functional group. A similar conjugation method is used for the folate-S,S-nigericin-S,S-rhodamine dual conjugate. In another illustrative example, a folate ligand is conjugated via a disulfide linkage to the hydroxyl group to form a folate-nigericin conjugate.

miRNA Duplexes can be constructed using two RNA oligonucleotides: denoted as miR-34a-5p guide strand and miR-34a-3p passenger strand. In some embodiments, the miR-34a-3p passenger strand comprises a 20 nt RNA oligo double modified with an azide linker on the 5′ end and 2′-O-methyl RNA bases (labeled as m) in positions 1, 2, 4, 6, 8, 10, 12, 14, 16 and 18, and the miR-34a-5p guide strand comprises a 22 nt RNA oligo with a phosphate group on the 5′ end and 2′-O-methyl RNA bases on the 3′ in positions 20 and 21.

It will be appreciated that that lentiviral- and liposomal-mediated delivery of tumor suppressive miRNA, miR-34a, reduces tumor burden in various non-small cell lung cancer (NSCLC) mouse models. It will also be appreciated that in addition to vehicle- and viral-mediated miRNA delivery, systemic injection of naked oligonucleotides has also been tested, and can be problematic. Without being bound by theory, it may be that pharmacokinetic and stability limitations associated with intravenous delivery requires reliance either on local delivery or achieving a high oligonucleotide concentration that is often only seen in kidneys and liver. In some embodiments, local delivery can be an option. In some embodiments, achieving delivery beyond sites that are accessible to local delivery, such as to micrometastatic lesions, can be achieved using a conjugate of the present disclosure.

In some embodiments, overcoming the challenges of non-targeted delivery can be achieved by applying conjugates of cell-surface receptors that are specifically overexpressed on tumor cells. In some embodiments, conjugates of the present disclosure can be applied to provide miRNA mimic delivery beyond sites accessible by local delivery. In some embodiments, a ligand that binds to a cell-surface receptor can be conjugated to a functionally active miRNA, and the resulting molecule can be used to target miRNAs specifically to tumor cells. In some embodiments, the target receptor can be a folate receptor (FR). Folate receptors are known to be overexpressed on the cancer cell relative to normal cells, and the expression level of the receptor must be sufficient to enable delivery of therapeutic quantities of a miRNA to the cancer cell. The folate receptor (FR) is known to be overexpressed on many epithelial cancers, including cancers of the breast, lung, ovary, kidney, and colon, and various hematological malignancies such as acute myeloid leukemia.

In contrast, the presence of the FR on normal tissues appears to be limited in quantity, inconsequential for targeted drug applications, or inaccessible to blood-borne folates.

In some embodiments, the binding ligand can be the FR ligand, Vitamin B9 (folic acid), that binds to the FR with high binding affinity, is selective for the FR, and contains a derivatizable functional group for facile conjugation to imaging or therapeutic agents that does not interfere with binding to the receptor. In some embodiments, FR/folate-conjugate therapy is provided herein for delivery of small RNAs such as miRNA or siRNA.

Successful folate-targeted delivery, with payloads as diverse as small radiopharmaceutical agents to large DNA-containing formulations, has been exemplified both at the preclinical and clinical levels. However, folate-mediated delivery of small RNAs lags behind due to the hypothesis that RNAs in circulation need to be protected. To achieve this level of protection, various strategies pursued in the field of small RNA delivery have incorporated folate onto a carrier vehicle (dendrimer, copolymer, liposome). These complexes can have a very large size, which often leads to hampered penetration of target tissues due to the dense extracellular matrix found in most solid tumors. Herein, we provide evidence for conjugates that directly link miRNA mimics to the folate ligand, which we have termed FolamiRs. Without being bound by theory it may be that the FolamiRs described herein perfuse solid tumors more easily than larger miRNA encapsulating vehicles. One possible difficulty is that the native form of small RNAs are relatively unstable in blood. In some embodiments, conjugates of the present disclosure comprise a passenger strand of the miRNA mimic that is minimally modified with 2′-O-methyl RNA bases, which may stabilize the RNA and possible increase nuclease resistance without impairing Argonaute loading.

It will be appreciated that folate linked to rhodamine saturates a solid tumor after i.v. injection in less than five minutes. The speed by which the folate-conjugated molecules enter the tumor demonstrates that FolamiRs described herein need only to survive in circulation for a very short period of time.

In some embodiments, the present disclosure provides a method for delivering functional and virtually unprotected miRNAs specifically and rapidly to tumor tissue. It is demonstrated herein that miRNA-34a (miR-34a) can be selectively targeted to a tumor, enter tumorigenic cells, can downregulate target gene, and can suppress growth of tumors in vivo. In some embodiments, fast tumor uptake that is mediated by directly conjugating miR-34a to folate (FolamiR-34a) can be beneficial.

The invention described herein also includes pharmaceutical compositions comprising the ligand-ionophore conjugate described herein and further comprising at least one pharmaceutically acceptable carrier or excipient. The ligand-ionophore conjugate is preferably administered to the patient (i.e., subject in need thereof) parenterally, e.g., intradermally, subcutaneously, intramuscularly, intraperitoneally, intravenously, or intrathecally. Alternatively, the ligand-ionophore conjugate can be administered to a patient (e.g., human or animal) by other medically useful processes, such as by inhalation, nasal administration, buccal absorption, transdermal, rectal or vaginal suppository, per os (oral), and any effective dose and suitable dosage form, including prolonged release dosage forms, can be used.

Examples of parenteral dosage forms include aqueous solutions of the ligand-ionophore conjugate in an isotonic saline solution, a glucose solution or other well-known pharmaceutically acceptable liquid carriers such as liquid alcohols, glycols, esters, and amides or suspensions of liposomes. The parenteral dosage form in accordance with this invention can be in the form of a reconstitutable lyophilizate comprising the dose of the ligand-ionophore conjugate. In one embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

The ligand-ionophore conjugate can be administered to the patient prior to, after, or at the same time as the therapeutic agent, or imaging agent that is internalized by endocytosis, as determined by the relevant medical professional.

### EXAMPLES

The following examples further illustrate specific embodiments of the invention; however, the following illustrative examples should not be interpreted in any way to limit the invention. Abbreviations used herein include: DCC, dicyclohexylcarbodiimide; Py, 2-pyridyl; RT, room temperature.

### Preparative Examples

### Materials

Amino acids for peptide synthesis were purchased from Aapptec, USA. N-Hydroxybenzotriazole (HOBt), 1-[Bis(dimethylamino)methylene]-1H-1,2,3-triazolo[4,5-b]pyridinium 3-oxid hexafluorophosphate (HATU) and benzotriazol-1-yloxytris(pyrrolidino)phosphonium hexafluorophosphate) (PyBOP) were obtained from Sigma-Aldrich. Solid phase peptide synthesis (SPPS) was performed using a standard peptide synthesis apparatus (Chemglass, Vineland, N.J.). Unless otherwise specified, all other chemicals were purchased from Sigma-Aldrich. All folate conjugates were purified by preparative reverse phase (RP)-HPLC (Agilent) and LC/MS analyses were obtained using a Agilent mass spectrometer coupled with a UV diode array detector. For avoidance of doubt, in each of the representations of compounds provided below where the compound is shown in a split view, it will be appreciated that a covalent attachment occurs between the two open bonds depicted as “-” on the parts of the compound. For example,

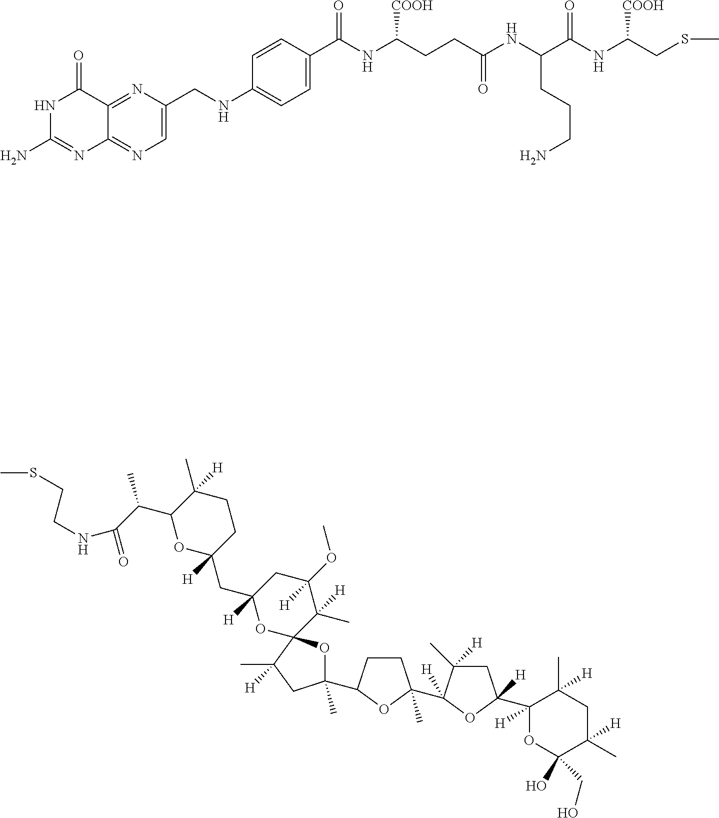

represents

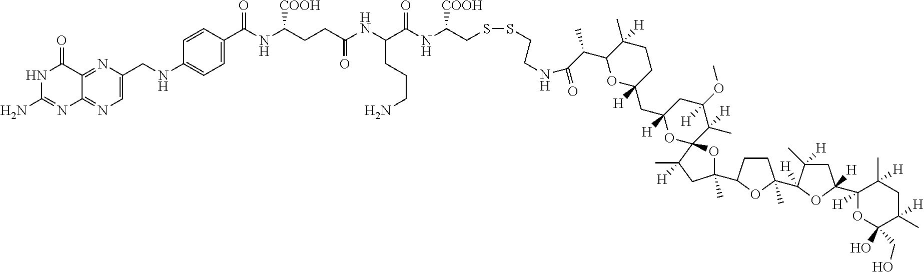

### Synthesis of Folate-EDA Conjugate

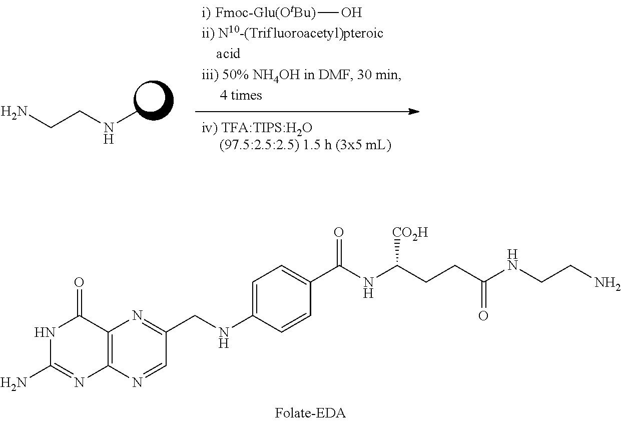

In a peptide synthesis vessel ethylenediamine, polymer-bound (200-400 mesh)-resin (1.000 g, 0.17 mmol, 1. eq.) was loaded and swollen with dichloromethane (3 mL) followed by dimethylformamide (3 mT) for 1 h. To the vessel was then introduced the Fmoc-Glu-OtBu solution (0.1808 g, 0.425 mmol, 2.5. eq.) in DMF, N,N-Diisopropylethylamine (DIPEA-i-Pr₂Net, 0.2202 g, 1.7 mmol, 10. eq.) and (Benzotriazol-1-yloxy)tripyrrolidinophosphonium hexafluorophosphate (PyBOP, 0.2212 g, 0.43 mmol, 2.5. eq.). Argon was bubbled for 4h, the coupling solution was drained, and the resin was washed with DMF (3×10 mL) and isopropanol (i-PrOH) (3×10 mL). Kaiser tests were performed to assess reaction completion. Fmoc deprotection was carried out using 20% piperidine in DMF (3×10 mL), before each amino acid coupling. The above sequence was repeated to complete reaction with Tfa.Pteroic-acid. At the end the resin was washed with 50% ammonium hydroxide in DMF 3×10 mL (5 min) to cleave the trifluoro-acetyl protecting group on pteroic acid and washed with i-PrOH (3×10 mL) followed by DMF (3×10 mL). The resin was dried under argon for 30 min. Folate-EDA peptide was cleaved from the resin using a cleavage mixture consisting of 95% CF₃COOH, 2.5% H₂O and 2.5% triisopropylsilane. 10 ml of the cleavage mixture was introduced and argon was bubbled for 1.5 h. The cleavage mixture was drained into a clean flask. The resin was washed 3 times with more cleavage mixture. The combined mixture was concentrated under reduced pressure to a smaller volume (˜5 mL) and precipitated by adding cold ethyl ether. The precipitate was collected by centrifugation, washed with ethyl ether (3 times) and dried under high vacuum. Crude reaction mixture was purified by RP-HPLC, (mobile phase A=10 mM ammonium acetate, pH=7; organic phase B=acetonitrile; method: 0% B to 30% B in 35 minutes at 13 ml/min) and furnished folate-EDA in 65% yield. LC-MS (A=10 mM ammonium bicarbonate, pH=7; organic phase B=acetonitrile; method: 0% B to 30% B in 12 minutes) R_{T}=3.26 min (M+H⁺=484.0).

### Synthesis of Folate-DBCO Conjugate

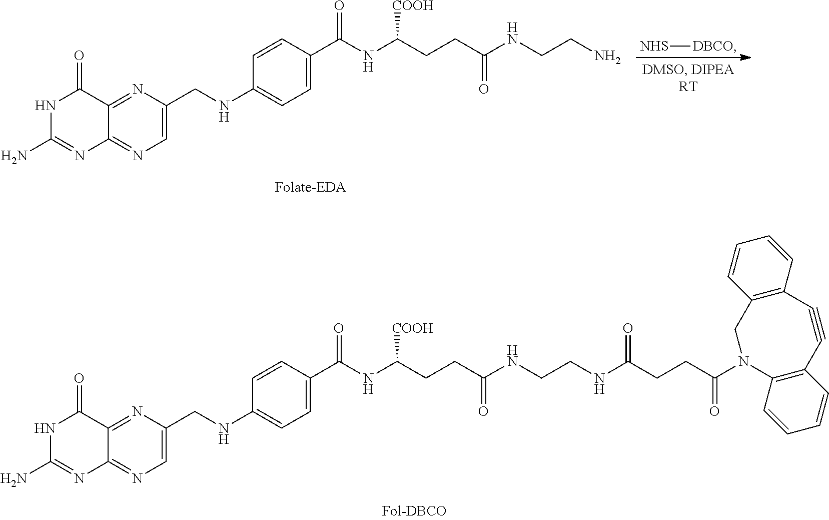

To a stirred solution of Folate-EDA (0.0100 g, 0.0206 mmol, 1 eq.) and NHS-DBCO (0.0091 g, 0.0227 mmol, 1.1 eq.) in DMSO, DIPEA (0.0039 g, 0.0309 mmol, 1.5 eq.) was added dropwise. The reaction mixture continued for stirring at room temp. Progress of the reaction was monitored by LCMS. After complete conversion of Folate-EDA the crude reaction mixture was purified by RP-HPLC. (mobile phase A=10 mM ammonium acetate, pH=7; organic phase B=acetonitrile; method: 0% B to 50% B in 35 minutes at 13 ml/min) and furnished Folate-DBCO in 85% yield. LC-MS (A=10 mM ammonium bicarbonate, pH=7; organic phase B=acetonitrile; method: 0% B to 50% B in 12 minutes) R_{T}=4.62 min (M+H⁺=771.0)

### Synthesis of Folate-SS-DBCO Conjugate

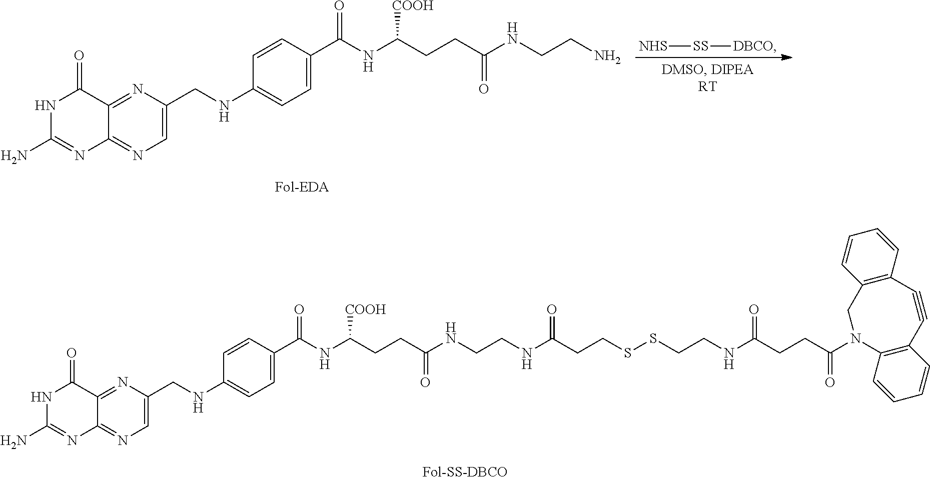

To a stirred solution of Folate-EDA (0.0100 g, 0.0206 mmol, 1 eq.) and NHS-SS-DBCO (0.0128 g, 0.0227 mmol, 1.1 eq.) in DMSO, DIPEA (0.0039 g, 0.0309 mmol, 1.5 eq.) was added dropwise. The reaction mixture continued for stirring at room temp. Progress of the reaction was monitored by LCMS. After complete conversion of Folate-EDA the crude reaction mixture was purified by RP-HPLC. (mobile phase A=10 mM ammonium acetate, pH=7; organic phase B=acetonitrile; method: 0% B to 50% B in 35 minutes at 13 ml/min) and furnished Folate-SS-DBCO in 82% yield. LC-MS (A=10 mM ammonium bicarbonate, pH=7; organic phase B=acetonitrile; method: 0% B to 50% B in 12 minutes) R_{T}=5.4 min (M+H⁺=934.0)

### Synthesis of Folate-Cys Conjugate

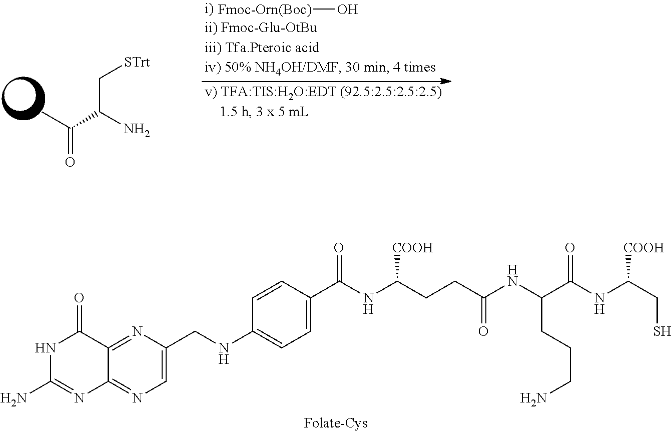

In a peptide synthesis vessel H-Cys(Trt)-2-Cl-Trt-resin (100-200 mesh, 0.200 g, 0.088 mmol, 1 eq.) was loaded and swollen with dichloromethane (3 mL) followed by dimethylformamide (3 mL) for 1 h. To the vessel was then introduced the Fmoc-Orn(Boc)-OH solution (0.080 g, 0.176 mmol, 2.0 eq.) in DMF, i-Pr₂NEt (0.0684 g, 0.528 mmol, 6.0 eq.), and PyBOP (0.1832 g, 0.35 mmol, 4.0 eq.). Argon was bubbled for 4h, the coupling solution was drained, and the resin was washed with DMF (3×10 mL) and i-PrOH (3×10 mL). Kaiser tests were performed to assess reaction completion. Fmoc deprotection was carried out using 20% piperidine in DMF (3×10 mL), before each amino acid coupling. The above sequence was repeated to complete reaction with Fmoc-Glu-OtBu (0.0749 g, 0.176 mmol, 2.0 eq.) and Tfa.Pteroic-acid (0.0359 g, 0.088 mmol, 1.0 eq.) coupling steps. At the end the resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min. Folate-Cys peptide was cleaved from the resin using a cleavage mixture consisting of 92.5% CF₃COOH, 2.5% H₂O, 2.5% ethanedithiol and 2.5% triisopropylsilane. 10 ml of the cleavage mixture was introduced and argon was bubbled for 1.5 h. The cleavage mixture was drained into a clean flask. The resin was washed 3 times with more cleavage mixture. The combined mixture was concentrated under reduced pressure to a smaller volume (˜5 mL) and precipitated in ethyl ether. The precipitate was collected by centrifugation, washed with ethyl ether (3 times) and dried under high vacuum. Crude reaction mixture was purified by RP-HPLC, (mobile phase A=10 mM ammonium acetate, pH=7; organic phase B=acetonitrile; method: 0% B to 30% B in 35 minutes at 13 ml/min) and furnished Folate-Cys 72% yield. LC-MS (A=10 mM ammonium bicarbonate, pH=7; organic phase B=acetonitrile; method: 0% B to 30% B in 12 minutes) R_{T}=3.73 min (M+H⁺=659.0).

Preparation of Pyridyldisulfide Amide Derivative of Nigericin

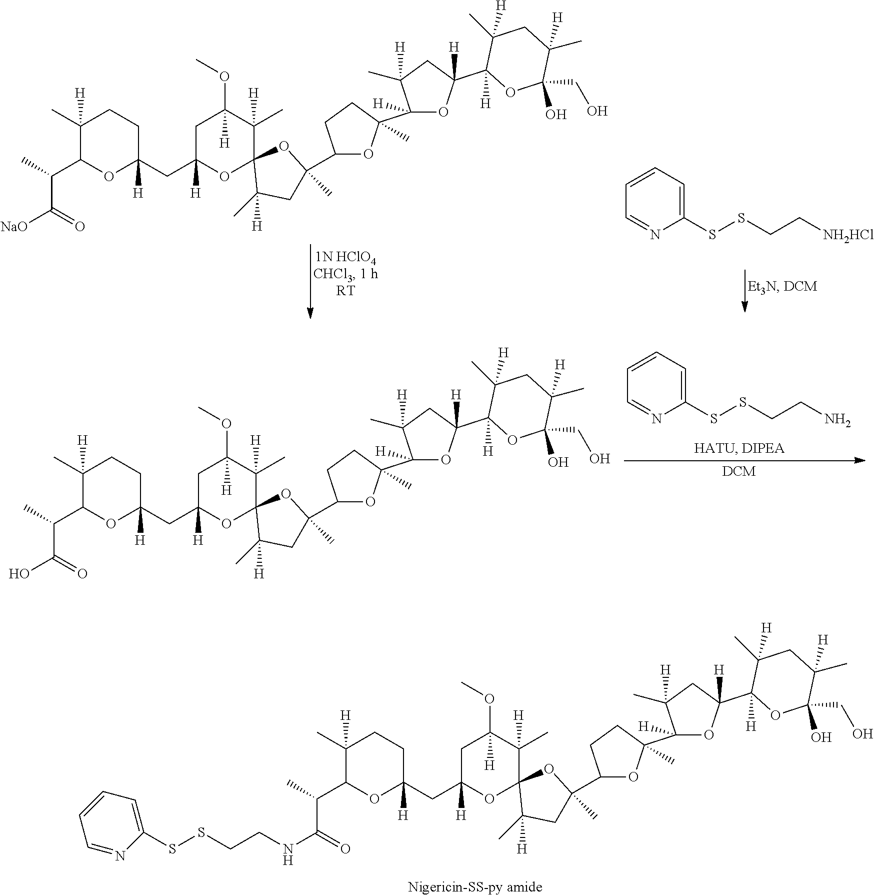

Nigericin free acid (0.035 mmol), Py-SS—(CH₂)₂NH₂ (0.052 mmol), HATU (0.052 mmol), and DIPEA (0.069 mmol) were dissolved in anhydrous CH₂Cl₂ (2.0 mL) and stirred under argon at room temperature overnight. Progress of the reaction was monitored by LCMS. After complete conversion of nigericin free acid, the crude reaction mixture was subjected to purification by R-HPLC, (mobile phase A=10 mM ammonium acetate, pH=7; organic phase B=acetonitrile; method: 0% B to 100% B in 35 minutes at 13 ml/min) and furnished nigericin-SS-amide derivative 55% yield. LC-MS (A=10 mM ammonium bicarbonate, pH=7; organic phase B=acetonitrile; method: 0% B to 100% B in 12 minutes) R_{T}=7.53 min (M+Na⁺=910.5)

### Preparation of Folate-Pyridyldisulfide Amide Derivative of Nigericin

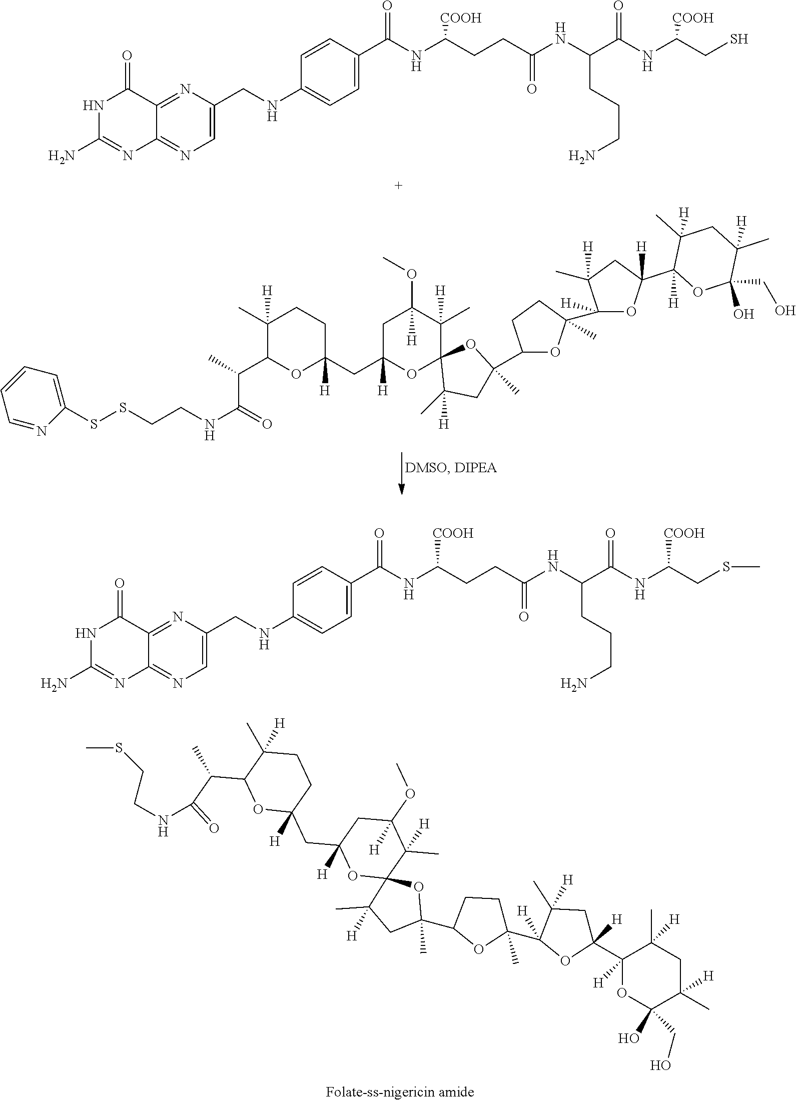

To a stirred solution of Folate-Cys (0.004 g, 0.007 mmol, 1.5 eq.) and Pyridyldisulfide amide derivative of Nigericin (0.004 g, 0.005 mmol, 1.0 eq.)) in DMSO, DIPEA was added dropwise. The reaction mixture continued for stirring at room temp. Progress of the reaction was monitored by LCMS. After complete conversion of Folate-Cys the crude reaction mixture was purified by RP-HPLC, (mobile phase A=10 mM ammonium acetate, pH=7; organic phase B=acetonitrile; method: 0% B to 40% B in 35 minutes at 13 ml/min) and furnished Folate-Pyridyldisulfide amide derivative of Nigericin 65% yield. LC-MS (A=10 mM ammonium bicarbonate, pH=7; organic phase B=acetonitrile; method: 0% B to 100% B in 12 minutes) R_{T}=4.2 min (M+H⁺=1441.0)

### Preparation of Folate-DBCO-Pyridyldisulfide Amide Derivative of Nigericin (Non-Releasable Conjugate)

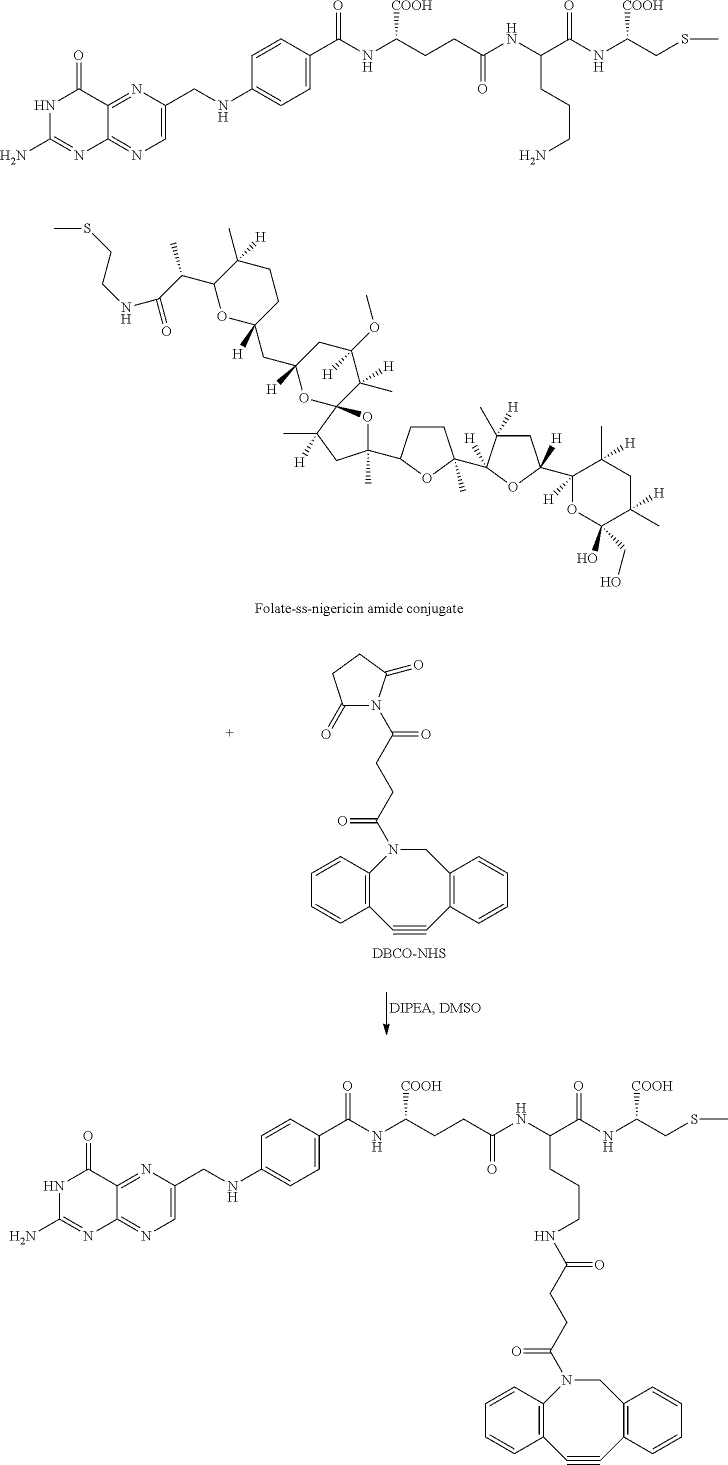

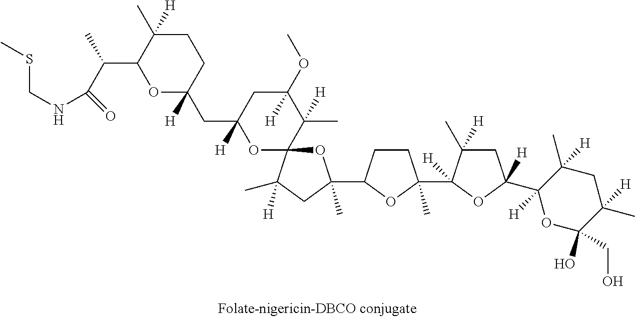

To a stirred solution of Folate-Pyridyldisulfide amide derivative of Nigericin (0.010 g, 0.0069 mmol, 1 eq.) and NHS-DBCO (0.003 g, 0.0076 mmol, 1.5 eq.) in DMSO, DIPEA (0.0013 g, 0.010 mmol, 1.5 eq.) was added dropwise. The reaction mixture continued for stirring at room temp. Progress of the reaction was monitored by LCMS. After complete conversion of Folate-SS-nigericin, the crude reaction mixture was purified by RP-HPLC, (mobile phase A=10 mM ammonium acetate, pH=7; organic phase B=acetonitrile; method: 0% B to 50% B in 35 minutes at 13 ml/min) and furnished Folate-DBCO-nigericin 65% yield. LC-MS (A=10 mM ammonium bicarbonate, pH=7; organic phase B=acetonitrile; method: 0% B to 100% B in 12 minutes) RT=5.38 min (M+H⁺=1728.4)

### Preparation of Folate-SS-DBCO-Pyridyldisulfide Amide Derivative of Nigericin (Releasable Conjugate)

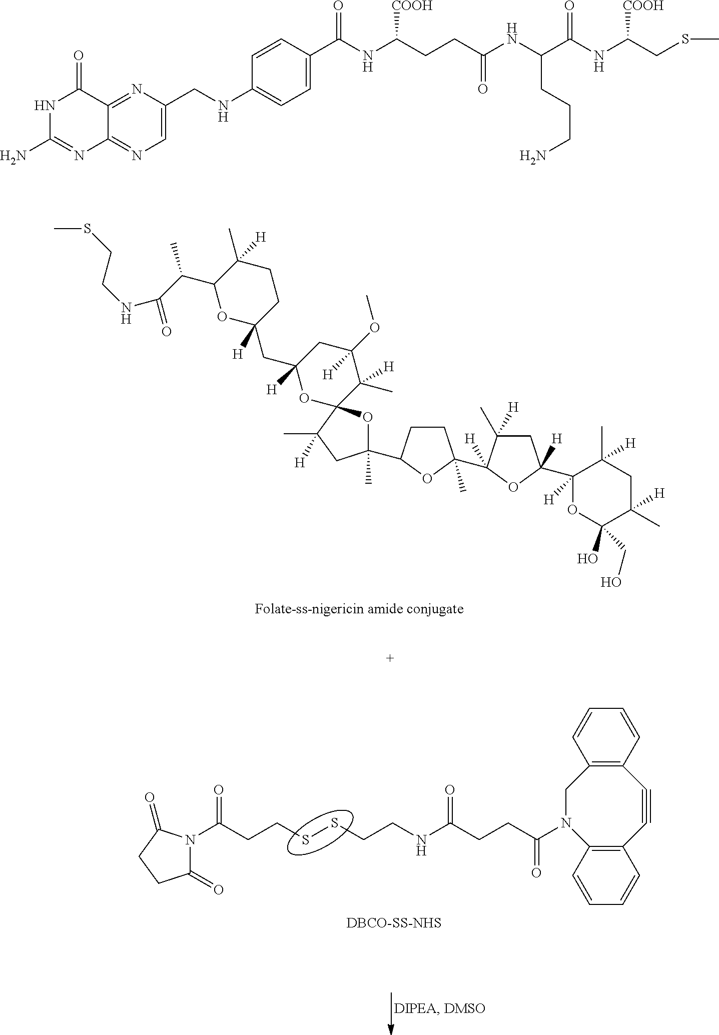

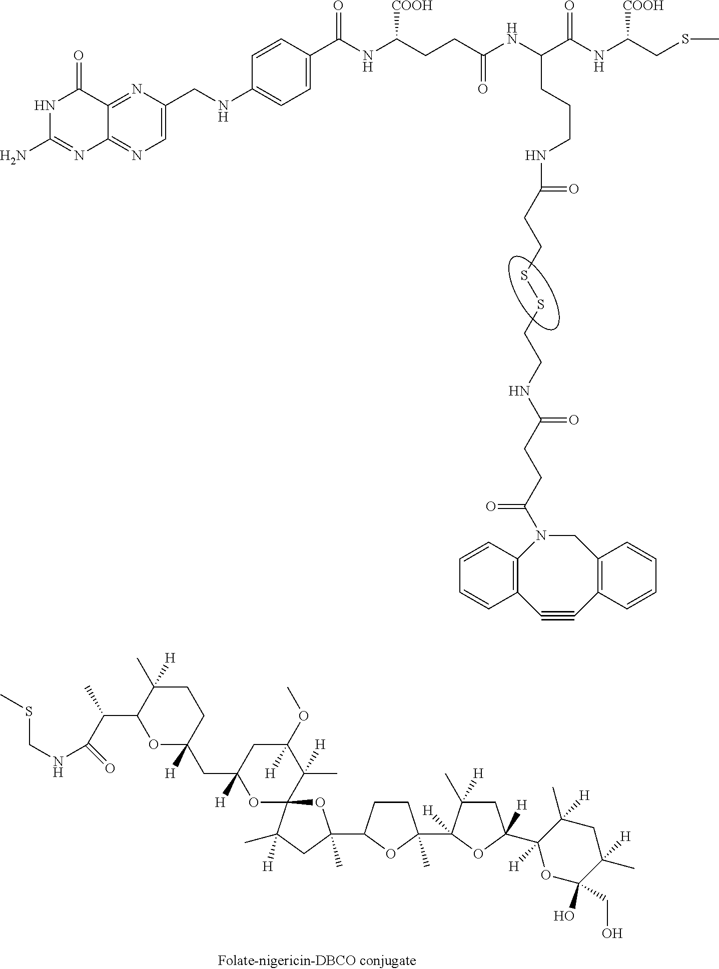

To a stirred solution of Folate-Pyridyldisulfide amide derivative of Nigericin (0.010 g, 0.0069 mmol, 1 eq.) and NHS-SS-DBCO (0.005 g, 0.010 mmol, 1.5 eq.) in DMSO, DIPEA (0.0013 g, 0.010 mmol, 1.5 eq.) was added dropwise. The reaction mixture continued for stirring at room temp. Progress of the reaction was monitored by LCMS. After complete conversion of Folate-SS-nig the crude reaction mixture was purified by RP-HPLC, (mobile phase A=10 mM ammonium acetate, pH=7; organic phase B=acetonitrile; method: 0% B to 50% B in 35 minutes at 13 ml/min) and furnished Folate-SS-DBCO-nigericin 65% yield. LC-MS (A=10 mM ammonium bicarbonate, pH=7; organic phase B=acetonitrile; method: 0% B to 100% B in 12 minutes) RT=6.52 min (M/2+H+=945.4)

### Preparation of Folate-Nigericin-miR-34a Conjugate

Folate-Nigericin DBCO miR-34a Conjugate:

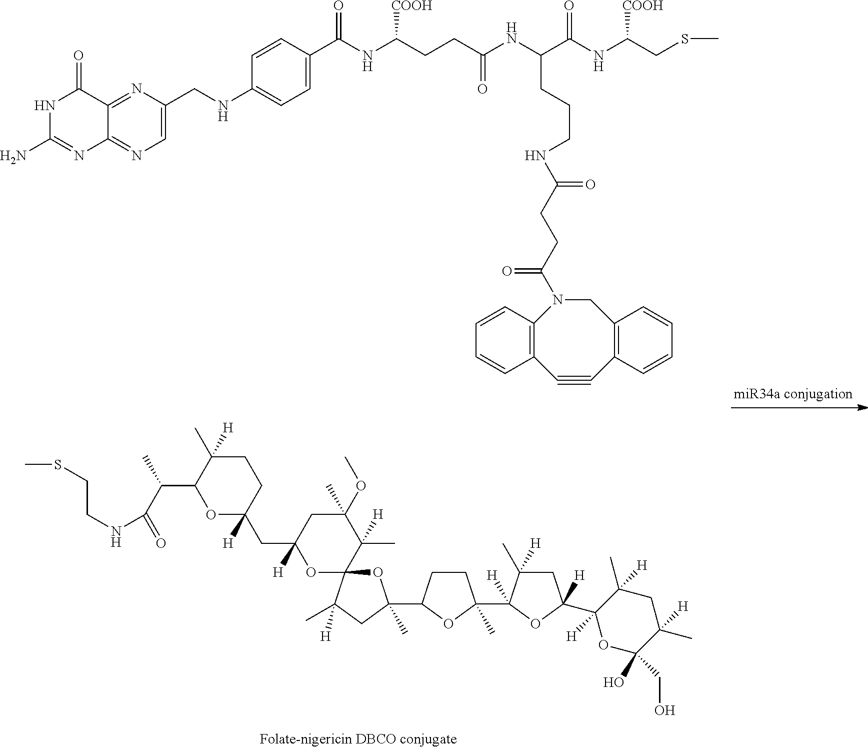

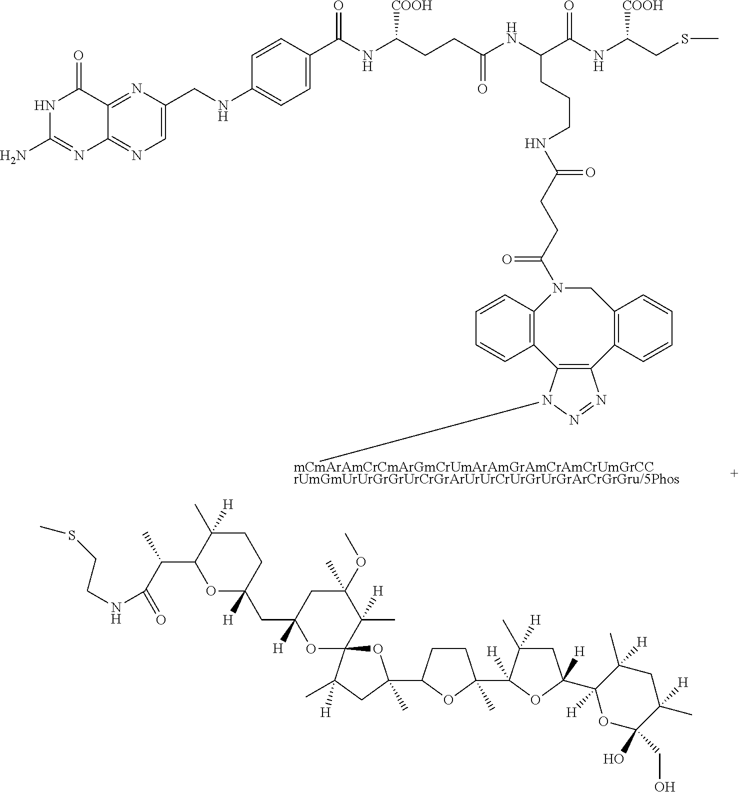

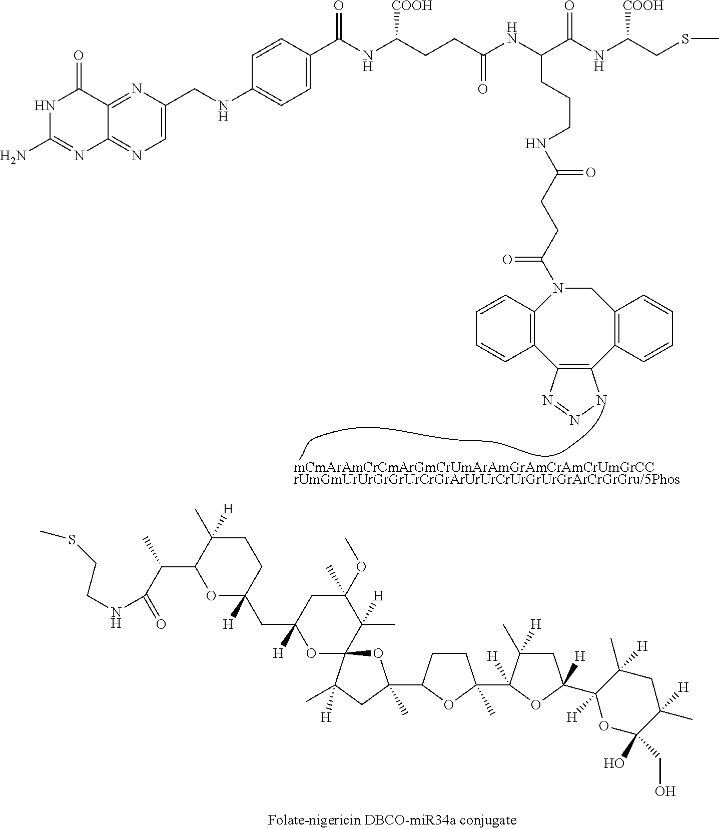

Folate-Nigericin-SS-DBCO miR-34a Conjugate:

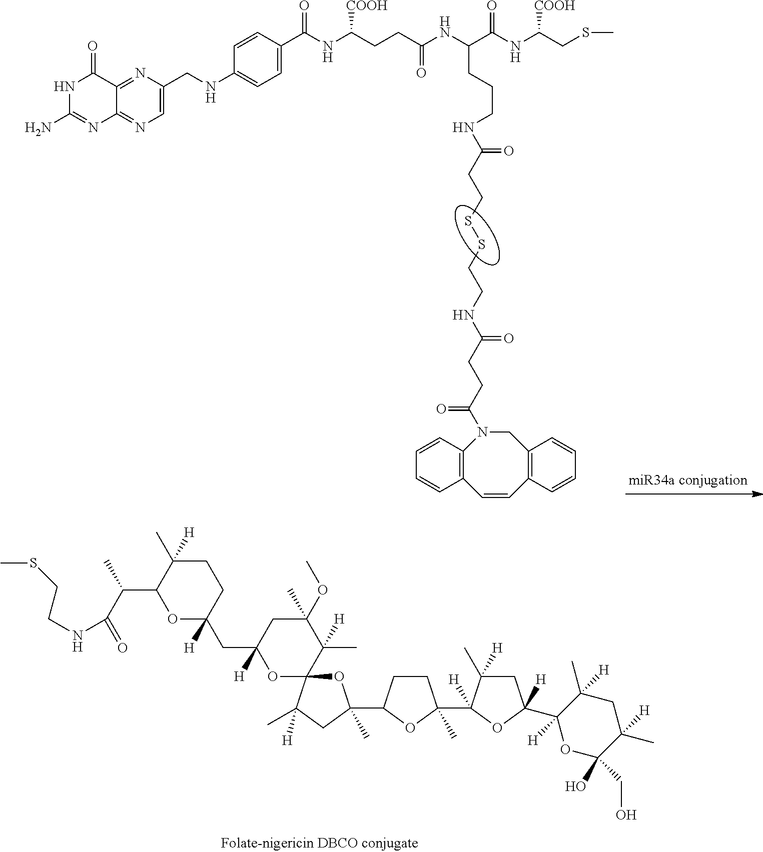

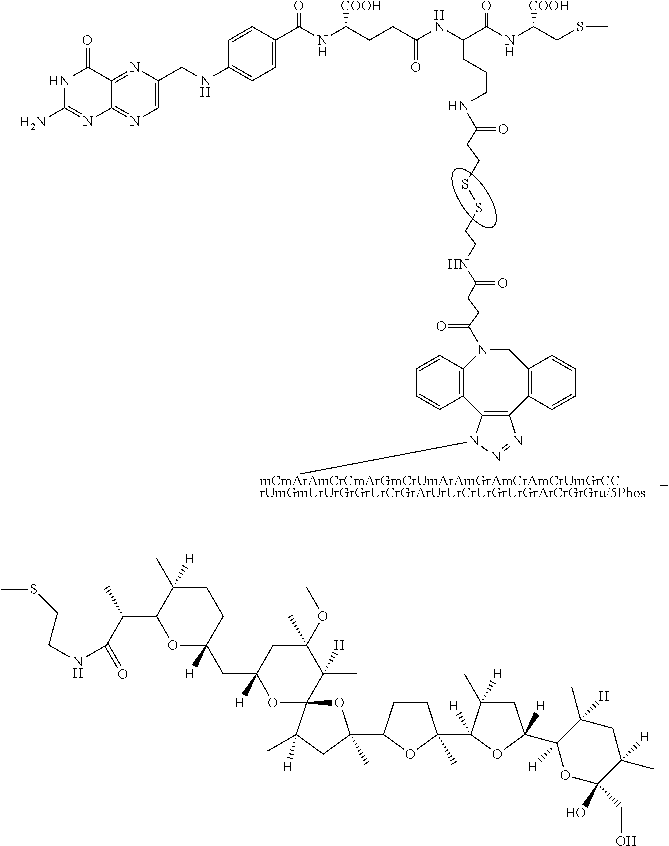

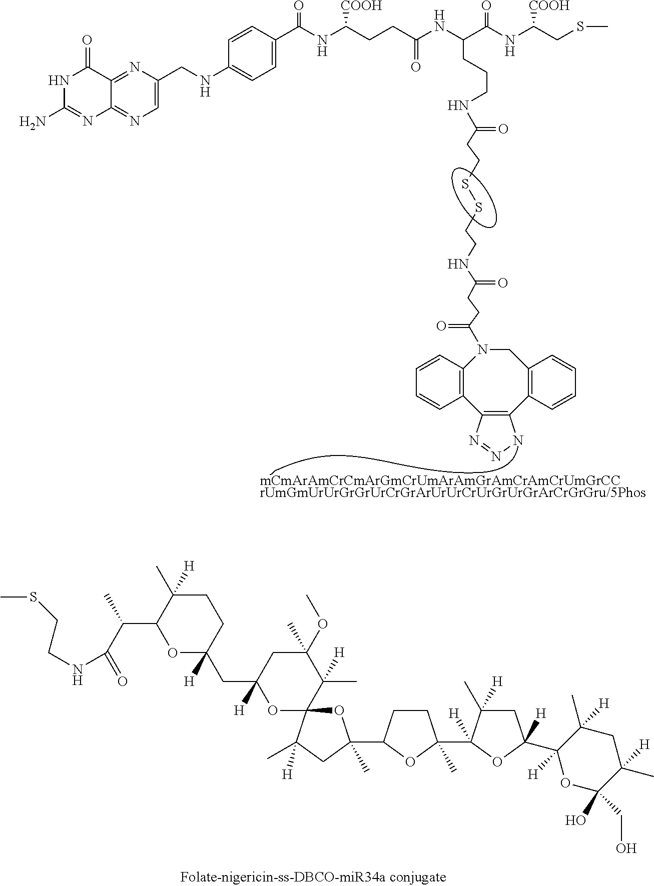

MiRNA duplexes were constructed using two RNA oligonucleotides: denoted as miR-34a-5p guide strand and miR-34a-3p passenger strand (both prepared by Integrated DNA Technologies). The miR-34a-3p passenger strand comprises a 20 nt RNA oligo double modified with an azide linker on the 5′ end and 2′-O-methyl RNA bases on the 3′ end (mCmArAmCrCmArGmCrUmArAmGrAmCrAmCrUmGrCC), and the miR-34a-5p guide strand comprises a 22 nt RNA oligo with minimal modifications on the 3′ with 2′-O-methyl RNA bases (rUmGmUrUrGrGrUrCrGrArUrUrCrUrGrUrGrArCrGrGrU/5Phos). A scrambled miRNA (Negative control) synthesized with the same modifications was used to form a control duplex. A bi-orthogonal click reaction was performed between Folate-DBCO-nigericin or Folate-SS-DBCO-nigericin conjugate and azide modified antisense miR-34a (or scramble).

Click reaction was performed at a 1:10 molar ratio (azide oligo:Folate conjugate) at room temperature in water for eight hours and then cooled to 4° C. for four hours. Unconjugated folate was removed from the reaction using Oligo Clean and Concentrator (Zymo Research) per manufacturer instructions. Conjugation was verified using 15% TAE native PAGE and MALDI spectral analysis. After conjugation, the miR-34a-5p guide strand was annealed to the folate conjugates. Briefly, folate-miR-34a-3p and miR-34a-5p were mixed in an equal molar ratio (1:1, final concentration 5 μM each) in annealing buffer: 10 mM Tris buffer pH 7 (Sigma), supplemented with 50 mM NaCl (Sigma), and 1 mM EDTA (Sigma), and incubated at 95° C. for five minutes and then ramp cooled to room temperature over a period of one hour and then stored at −80° C.

Preparation of Folate-Nigericin-DBCO-siRNA Conjugates

Folate-Nigericin DBCO-siLuc2 Conjugate:

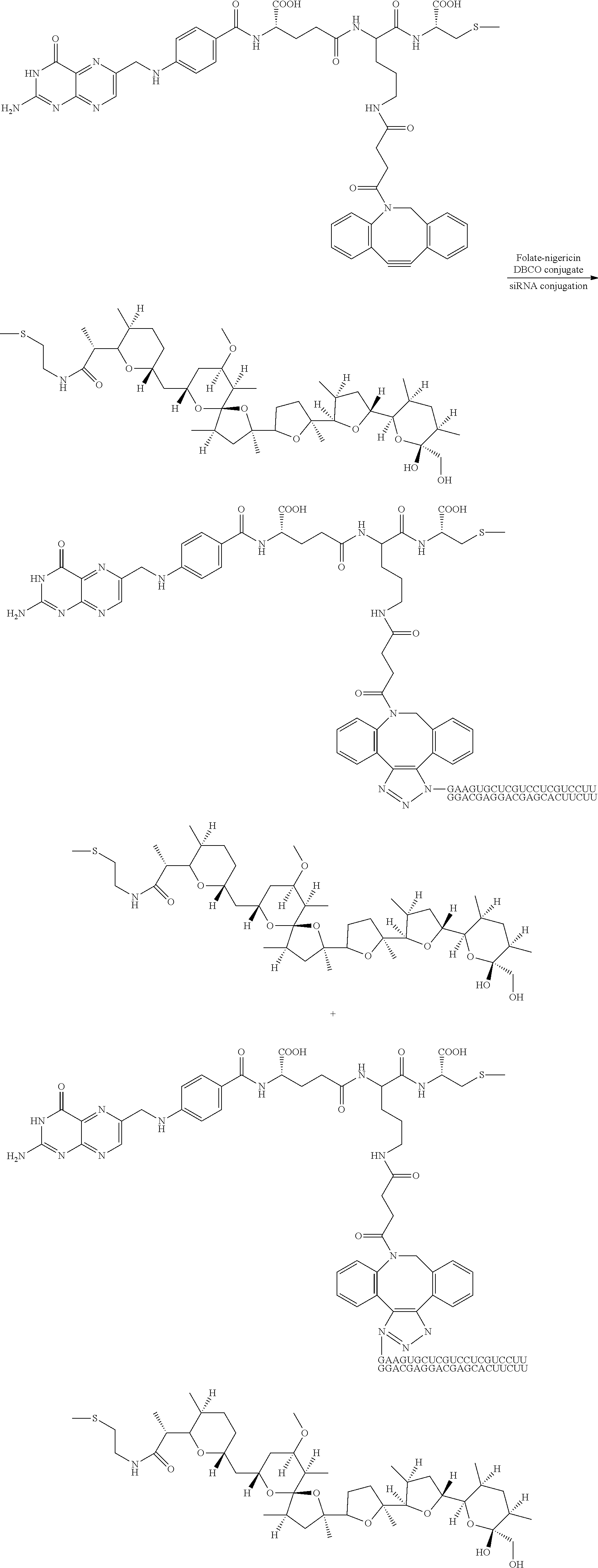

Folate-Nigericin-SS-DBCO-siLuc2 Conjugate:

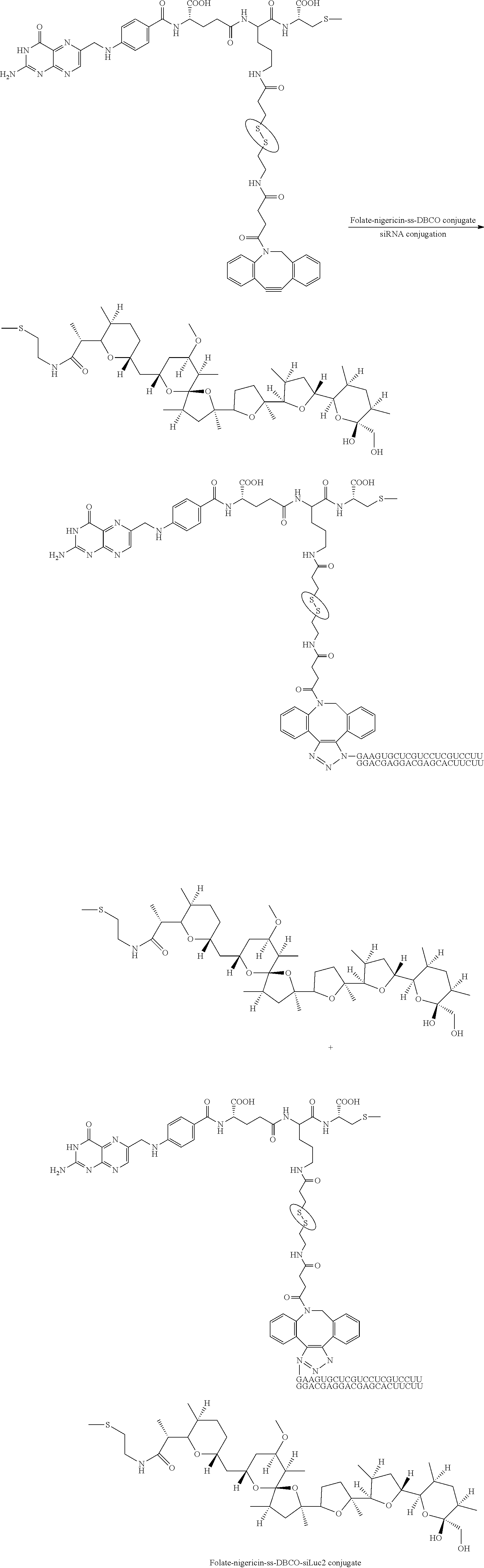

siRNA duplexes were constructed using two RNA oligonucleotides: denoted as siLuc2 sense strand (GGACGAGGACGAGCACUUCUU) and siLuc2 antisense strand (GAAGUGCUCGUCCUCGUCCUU) (Integrated DNA Technologies). A bi-orthogonal click reaction was performed between Folate-nigericin-DBCO or Folate-nigericin-ss-DBCO and azide modified antisense siRNA (or scramble). Click reaction was performed at a 1:10 molar ratio (azide oligo:Folate-nigericin-DBCO) at room temperature in water for eight hours and then cooled to 4° C. for four hours. Unconjugated folate was removed from the reaction using Oligo Clean and Concentrator (Zymo Research) per manufacturer instructions. Conjugation was verified using 15% TAE native PAGE and MALDI spectral analysis. After conjugation, the siLuc2 sense strand was annealed to the Folate-nigericin-DBCO-siRNA antisense conjugates. Briefly, folate-siLuc2 antisense and siLuc2 sense were mixed in an equal molar ratio (1:1, final concentration 5 μM each) in annealing buffer: 10 mM Tris buffer pH 7 (Sigma), supplemented with 50 mM NaCl (Sigma), and 1 mM EDTA (Sigma), and incubated at 95° C. for five minutes and then cooled slowly to room temperature and then stored at −80° C.

### Preparation of Folate-Cy5 Dye Conjugate

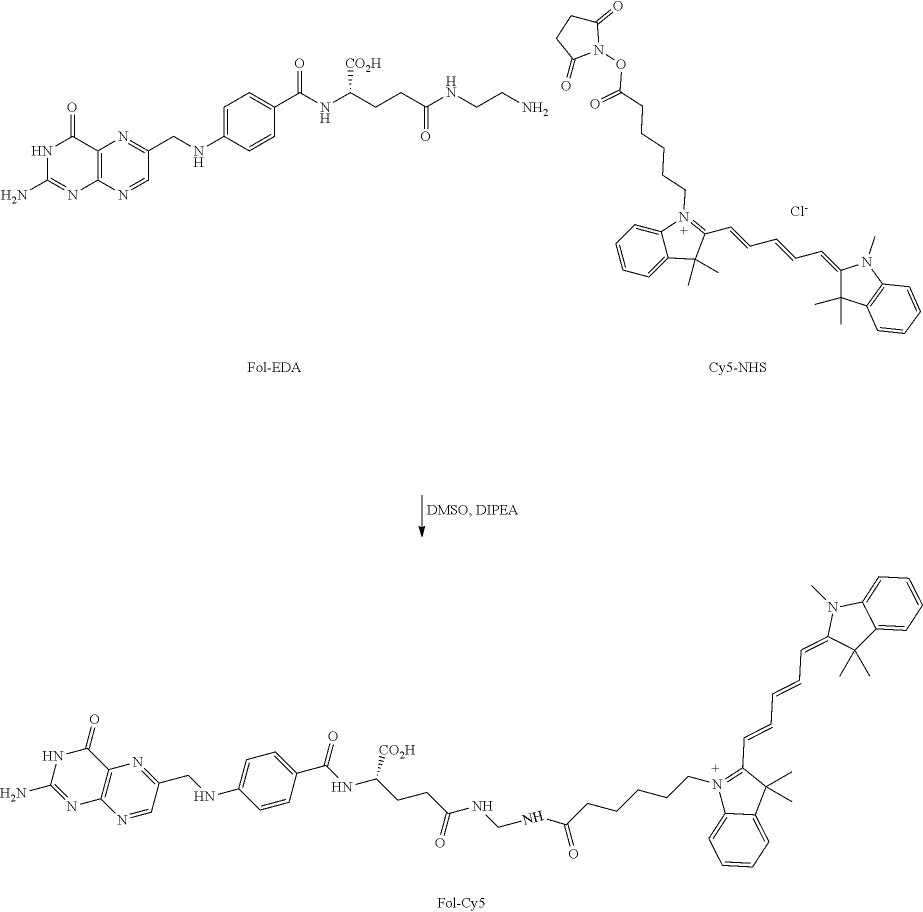

To a stirred solution of Folate-EDA (0.0008 g, 0.0016 mmol, 1 eq.) and NHS-Cy5 (0.001 g, 0.001 mmol, 1.1 eq.) in DMSO, DIPEA (0.0004 g, 0.0032 mmol, 2 eq.) was added dropwise. The reaction mixture continued for stirring at room temp. Progress of the reaction was monitored by LCMS. After complete conversion of Folate-EDA the crude reaction mixture was purified by RP-HPLC. (mobile phase A=10 mM ammonium acetate, pH=7; organic phase B=acetonitrile; method: 0% B to 50% B in 35 minutes at 13 ml/min) and furnished Folate-Cy5 dye conjugation 85% yield. LC-MS (A=10 mM ammonium bicarbonate, pH=7; organic phase B=acetonitrile; method: 0% B to 50% B in 12 minutes) RT=2.40 min (M+H⁺=949.2)

### Preparation of Folate-Cy5-Pyridyldisulfide Amide Derivative of Nigericin Dye Conjugate

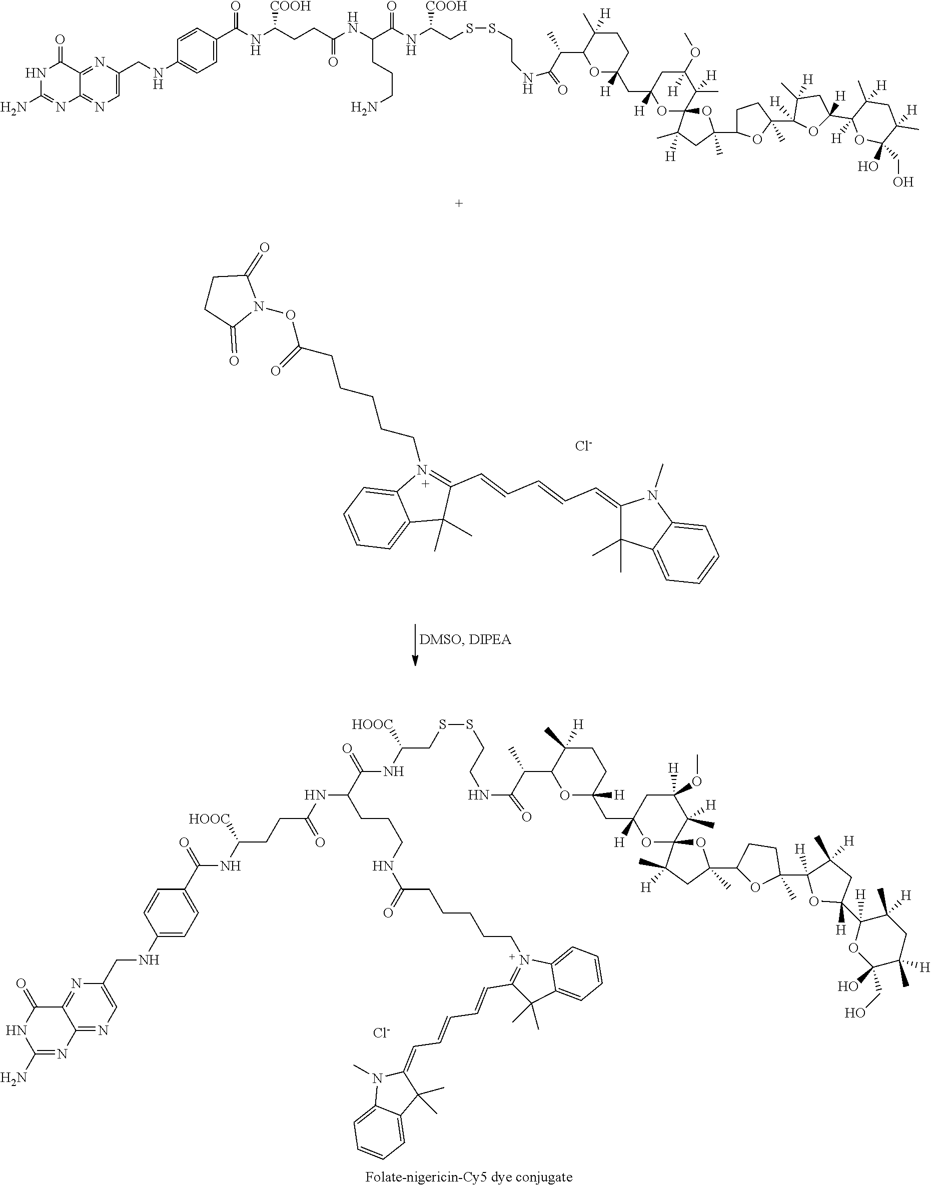

To a stirred solution of Folate-Pyridyldisulfide amide derivative of Nigericin (0.0023 g, 0.0016 mmol, 1 eq.) and NHS-Cy5 (0.001 g, 0.0016 mmol, 1 eq.) in DMSO, DIPEA (0.0004 g, 0.0032 mmol, 2. eq.) was added dropwise. The reaction mixture continued for stirring at room temp. Progress of the reaction was monitored by LCMS. After complete conversion of Folate-ss-nig the crude reaction mixture was purified by RP-HPLC, (mobile phase A=10 mM ammonium acetate, pH=7; organic phase B=acetonitrile; method: 0% B to 50% B in 35 minutes at 13 ml/min) and furnished Folate-nigericin-Cy5 65% yield. LC-MS (A=10 mM ammonium bicarbonate, pH=7; organic phase B=acetonitrile; method: 0% B to 100% B in 12 minutes) RT=7.05 min (M+^{H}=1906.0)

### Synthesis of Folate-NIR Dye Conjugate:

To a stirred solution of Folate-Cys (0.010 g, 0.015 mmol, 1 eq.)) and Maleimide-NIR Dye (0.019 g, mmol, 1.1 eq.)) in DMSO, DIPEA (0.0029 g, 0.0228 mmol, 1.5 eq.) was added dropwise. The reaction mixture continued with stirring at room temp. Progress of the reaction was monitored by LC-MS. After complete conversion of Folate-Cys the crude reaction mixture was purified by RP-HPLC, (mobile phase A=10 mM ammonium acetate, pH=7; organic phase B=acetonitrile; method: 0% B to 30% B in 35 minutes at 13 ml/min) and furnished Folate-NIR 85% yield. LC-MS (A=10 mM ammonium bicarbonate, pH=7; organic phase B=acetonitrile; method: 0% B to 30% B in 12 minutes) R_{T}=3.30 min (M+^{H}=1179.0).

### Synthesis of Folate-DBCO-NIR Dye Conjugate:

To a stirred solution of Folate-NIR dye (0.0050 g, 0.0027 mmol, 1 eq.) and NHS-DBCO (0.0016 g, 0.0041 mmol, 1.5 eq.) in DMSO, DIPEA (0.0054 g, 0.0041 mmol, 1.5 eq.) was added dropwise. The reaction mixture continued with stirring at room temp. Progress of the reaction was monitored by LC-MS. After complete conversion of Folate-NIR dye the crude reaction mixture was purified by RP-HPLC, (mobile phase A=10 mM ammonium acetate, pH=7; organic phase B=acetonitrile; method: 0% B to 50% B in 35 minutes at 13 ml/min) and furnished Folate-DBCO-NIR 86% yield. LC-MS (A=10 mM ammonium bicarbonate, pH=7; organic phase B=acetonitrile; method: 0% B to 50% B in 12 minutes) RT=4.75 min (M/2+H⁺=1043.0).

### Synthesis of Folate-SS-DBCO-NIR Conjugate:

To a stirred solution of Folate-NIR dye (0.0050 g, 0.0027 mmol, 1 eq.) and NHS-SS-DBCO (0.0023 g, 0.0041 mmol, 1.5 eq.) in DMSO, DIPEA (0.0054 g, 0.0041 mmol, 1.5 eq.) was added dropwise. The reaction mixture continued with stirring at room temp. Progress of the reaction was monitored by LCMS. After complete conversion of Folate-NIR dye the crude reaction mixture was subjected to purification by R-HPLC, (mobile phase A=10 mM ammonium acetate, pH=7; organic phase B=acetonitrile; method: 0% B to 50% B in 35 minutes at 13 ml/min) and furnished Folate-SS-DBCO-NIR 84% yield. LC-MS (A=10 mM ammonium bicarbonate, pH=7; organic phase B=acetonitrile; method: 0% B to 50% B in 12 minutes) RT=4.991 min (M/2+H⁺=1125.0).

### Preparation of Folate-miRNAs (FolamiRs)

MiRNA duplexes were constructed using two RNA oligonucleotides: denoted as miR-34a-5p guide strand and miR-34a-3p passenger strand (both prepared by Integrated DNA Technologies). The miR-34a-3p passenger strand comprises a 20 nt RNA oligo double modified with an azide linker on the 5′ end and 2′-O-methyl RNA bases on the 3′ end (mCmArAmCrCmArGmCrUmArAmGrAmCrAmCrUmGrCC), and the miR-34a-5p guide strand comprises a 22 nt RNA oligo with minimal modifications on the 3′ with 2′-O-methyl RNA bases (rUmGmUrUrGrGrUrCrGrArUrUrCrUrGrUrGrArCrGrGrU/5Phos). A scrambled miRNA (Negative control) synthesized with the same modifications was used to form a control duplex. A bi-orthogonal click reaction was performed between Folate-DBCO or Folate-SS-DBCO, and azide modified antisense miR-34a (or scramble). Click reaction was performed at a 1:10 molar ratio (azide oligo:Folate DBCO or Folate-SS-DBCO) at room temperature in water for eight hours and then cooled to 4° C. for four hours. Unconjugated folate was removed from the reaction using Oligo Clean and Concentrator (Zymo Research) per manufacturer instructions.

Conjugation was verified using 15% TAE native PAGE and MALDI spectral analysis. For folate-NIR compound conjugation an additional verification was done using Licor Odyssey CLX (Licor).

After conjugation, the miR-34a-5p guide strand was annealed to the folate and NIR-folate conjugates. Briefly, folate-miR-34a-3p and miR-34a-5p were mixed in an equal molar ratio (1:1, final concentration 5 μM each) in annealing buffer: 10 mM Tris buffer pH 7 (Sigma), supplemented with 50 mM NaCl (Sigma), and 1 mM EDTA (Sigma), and incubated at 95° C. for five minutes and then ramp cooled to room temperature over a period of one hour and then stored at −80° C.

### Stability Assay in Serum

The duplex RNA oligonucleotides and the FolamiR conjugates were incubated in 50% fetal bovine serum (Sigma) in water at 37° C. for the indicated times. RNA samples were collected and analyzed using 15% TAE polyacrylamide gel electrophoresis (PAGE).

TABLE 1
Abbreviations and Source Information

| Term | Description | Source |
|---|---|---|
| Calcein | Calcein dye | Life Technologies, Div. |
| | | of Fisher Scientific, |
| | | Pittsburgh, PA |
| | CHCl₃ | EMD Millipore, |
| | | Billerica, MA |
| | CH₂Cl₂ (anhydrous) | Sigma-Aldrich, St. |
| | | Louis, MO |
| | CH₃COOH | Sigma-Aldrich, St. |
| | | Louis, MO |
| | Diphosgene | Acros Organics, |
| | | distributed by Fisher |
| | | Scientific, Pittsburgh, |
| | | PA |
| DMSO | Dimethyl sulfoxide | Sigma-Aldrich, St. |
| | | Louis, MO |
| EC-119 | (2R,5S,8S,11S,14S,19S)-19-(4-(((2- | Endocyte, Inc., West |
| | amino-4-oxo-3,4-dihydropteridin-6- | Lafayette, IN |
| | yl)methyl)amino)benzamido)- | |
| | 5,8,14-tris(carboxymethyl)-11-(3- | |
| | guanidinopropyl)-2- | |
| | (mercaptomethyl)-4,7,10,13,16- | |
| | pentaoxo-3,6,9,12,15- | |
| | pentaazaicosane-1,20-dioicacid | |
| | HClO₄ | Sigma-Aldrich, St. |
| | | Louis, MO |
| DCC | N,N□-Dicyclohexylcarbodiimide | Alfa Aesar, Ward Hill, |
| | | MA |
| EtOAc | Ethyl Acetate | Sigma-Aldrich, St. |
| | | Louis, MO |
| FDRPMI | Folate-Deficient RPMI (Roswell | Sigma-Aldrich, St. |
| | Park Memorial Institute) Medium | Louis, MO |
| FR | Folate Receptor | |
| | HClO₄ | Sigma-Aldrich, St. |
| | | Louis, MO |
| LC-MS | Liquid Chromatography-Mass | Performed on a Waters |
| | Spectrometry | LC-MS system |
| | | (Milford, MA) with a |
| | | Waters Micromass ZQ |
| | | mass spectrometer; |
| | | Xbridge ™ Shield RP- |
| | | 18, 5 μm, 3.0 × 50 mm |
| | | column; flow rate of |
| | | 0.75 mL/min; mobile |
| | | phase of 20 mM |
| | | NH₄HCO₃ buffer, pH 7. |
| MeOH | Methanol | Sigma-Aldrich, St. |
| | | Louis, MO |
| | Na₂SO₄ | Mallinckrodt-Baker, |
| | | Phillipsburg, NJ |
| | Nigericin, sodium salt | A.G. Scientific, San |
| | | Diego, CA |
| Proton | Registered trademark for N, N, | Sigma-Aldrich, St. |
| Sponge^{□} | N□, N□-tetramethyl-1,8- | Louis, MO |
| | naphthalenediamine | |
| PyS- | 2-(2-pyridyldithio)-ethanol | Endocyte, Inc., West |
| S(CH₂)₂OH | | Lafayette, IN |
| | Pyridyldisulfide ethylamine HCl | Molecular Biosciences, |
| | | Boulder, CO |
| | Pyrrolidinopyridine | Sigma-Aldrich, St. |
| | | Louis, MO |
| RP-HPLC | Reversed-Phase High-Performance | Performed on a Waters |
| | Liquid Chromatography | RP-HPLC system |
| | | (Milford, MA); |
| | | XTerra^{□} Prep MS C18 |
| | | OBD ™ 50 μm, 19 × |
| | | 30 mm column; binary |
| | | gradient elution with |
| | | 10 mM |
| | | triethylammonium |
| | | acetate buffer, pH 7 |
| | | and methanol; flow |
| | | rate 26 mL/min; UV |
| | | detection 280 nm |
| RT | Room Temperature | |
| TLC | Thin-Layer Chromatography: Silica | EMD Millipore, |
| | Gel 60 F254 | Billerica, MA |
| | Triethylamine | Sigma-Aldrich, St. |
| | | Louis, MO |
| | Triethylammonium acetate | Sigma-Aldrich, St. |
| | | Louis, MO |
| TFA | Trifluoro acetic acid | Sigma-Aldrich, St. |
| | | Louis, MO |
| TIPS | Trisisopropylsilane | Sigma-Aldrich, St. |
| | | Louis, MO |
| DMF | N, N′-dimethylformamide | Sigma-Aldrich, St. |
| | | Louis, MO |
| DIPEA | N,N′-diisopropylethylamine | Sigma-Aldrich, St. |
| | | Louis, MO |
| HOBt | N-Hydroxybenzotriazole | Sigma-Aldrich, St. |
| | | Louis, MO |
| HATU | 1-[Bis(dimethylamino)methylene]- | Sigma-Aldrich, St. |
| | 1H-1,2,3-triazolo[4,5-b]pyridinium | Louis, MO |
| | 3-oxid hexafluorophosphate | |
| PyBOP | benzotriazol-1- | Sigma-Aldrich, St. |
| | yloxytris(pyrrolidino)phosphonium | Louis, MO |
| | hexafluorophosphate) | |
| Fol | Folate | Sigma-Aldrich, St. |
| | | Louis, MO |
| DBCO | dibenzocyclooctyne | Sigma-Aldrich, St. |
| | | Louis, MO |
| NHS | N-Hydroxysuccinimide | Sigma-Aldrich, St. |
| | | Louis, MO |
| Nig | nigericin | Sigma-Aldrich, St. |
| | | Louis, MO |
| MiR34a | microRNA-34a | Sigma-Aldrich, St. |
| | | Louis, MO |
| EDT | ethanedithiol | Sigma-Aldrich, St. |
| | | Louis, MO |

### Method Examples

### Cell Lines

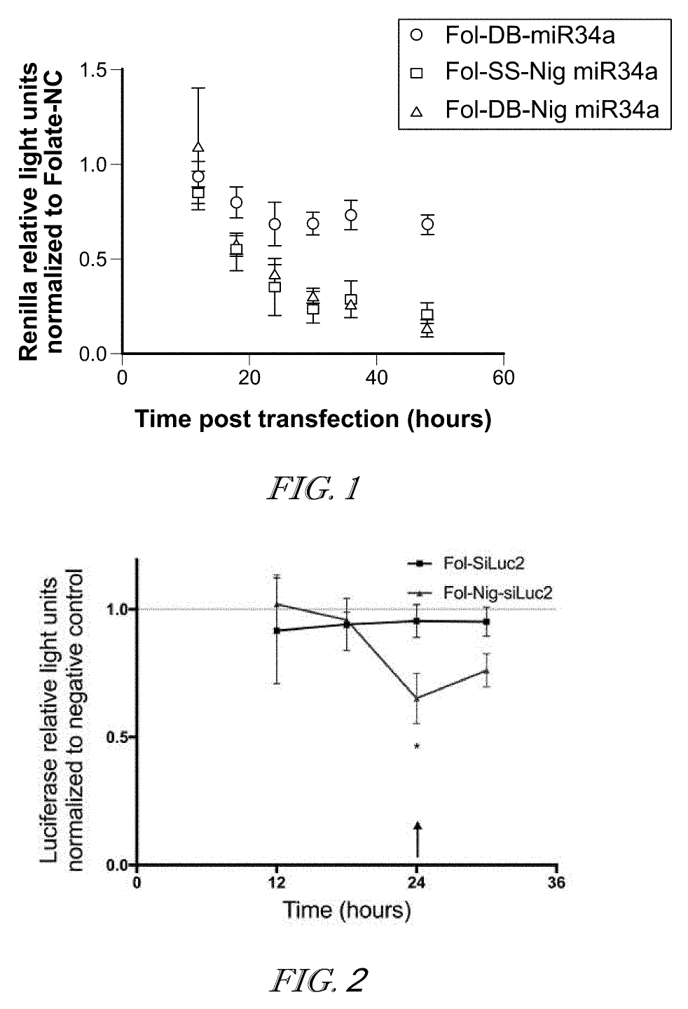

To monitor FolamiR-34a conjugates activity in cells MDA-MB-231 cells were generated that express a miR-34a Renilla sensor (MB-231 sensor). Firstly, specificity of the miRNA sensor was monitored by transiently expressing the miR-34a sensor or a mutated sensor along a miR-34a mimic or a negative control (scrambled RNA) in MDA-MB-231 breast cancer cells. For that purpose, 1×10⁴ cells were seeded in 96-well plates and co-transfected with 25 ng of plasmid and 6 nM of miRNA mimic using Lipofectamine 2000 (Life Technologies). Renilla activity was measured 48 hours post transfection using the Renilla Glo Luciferase kit (Promega). The results suggest that miR-34a is endogenously active in MB-231 cells and that delivery of exogenous miR-34a promotes further silencing of the Renilla sensor. These results suggest that the miR-34a sensor is specific to miR-34a and that the endogenous levels of miR-34a in MB-231 cells are not enough to fully silence the miR-34a sensor thus leaving some room to increase the knockdown with exogenous miR-34a. Stable clones were generated and tested for Renilla activity. Amongst 15 single stable clones, clone 5 was selected for further experiments due to high Renilla levels and its ability to monitor miR-34a activity. (FIG. 1)

In vitro Renilla Luciferase Activity

MDA-MB-231 triple-negative breast cancer cells (HTB-26, mycoplasma free, tested for mycoplasma contamination via MycoAlert Mycoplasma Detection Kit—Lonza) were grown in RPMI 1640 medium, no folic acid (Life Technologies) supplemented with 10% fetal bovine serum (Sigma), 100 U ml⁻¹ penicillin and 100 μg ml⁻¹ streptomycin (Hyclone, GE Healthcare Life Sciences) and maintained at 37° C. in 5% CO2. For luciferase reporter experiments, a miR-34a sensor plasmid was generated by inserting the antisense sequence to miR-34a into the 3′ untranslated region of Renilla luciferase in the vector (psiCHECK, Promega). MiR-34a specific silencing was confirmed in MDA-MB-231 cells by transiently transfecting a miR-34a sensor or a mutated miR-34a sensor. MiR-34a sensor expressing cells were transfected with a miR-34a mimic using Lipofectamine RNAimax (Life Technologies) to confirm silencing mediated by exogenous miRNA. To generate stable clones, MDA-MB-231 cells were seeded in six-well plates at a density of 1×10⁶ cells/well and were transfected with 2 ug of miR-34a sensor plasmid using Lipofectamine 2000 (Life Technologies). Stable clones were selected using Hygromycin B (500 g/mL; Hyclone, GE Healthcare Life Sciences) as a selection marker. Single clones were evaluated for Renilla expression and the clone with the highest Renilla expression was selected. MB-231 sensor cells were seeded into 96-well plates containing Folate-DBCO-miR-34a, Folate-nigericin-DBCO-miR34a, Folate-nigericin-SS-DBCO-miR34a and FolamiR-NC (negative control) in folic acid and serum free RPMI medium for a final concentration of 50 nM. Untreated and unconjugated duplex miRNA were included as controls. Renilla luciferase values were obtained between 12-48 h post incubation using the Renilla Glo Luciferase kit (Promega) following the manufacturer instructions. Renilla levels were normalized to FolamiR-NC for each time point. Experiments were performed three times with technical triplicates for each condition.

miR34a-Renilla Luciferase Gene Knockdown Activity

MB-231 sensor cells (in the absence of transfection reagent) treated with Folate-nigericin-DBCO-miR-34a or Folate-nigericin-SS-DBCO-miR-34a exhibits decrease in Renilla activity upto 80% after 48 h treatment (FIG. 1). However, Folate-DBCO-miR-34a lacking nigericin shows only 30% gene knockdown activity up to 48 h, which confirms nigericin helping for release of miR-34a from endosome. Surprisingly, both the releasable and unreleasable Folate-nigericin-miR-34a's efficiently entered the cell and retain activity based on the data, suggesting that the conjugated folate does not interfere with loading of miR-34a-5p into Argonaute.

siRNA Gene Knockdown Assay

For siRNA targeting assays, MDA-MB-231 cells were seeded in 6 well plates at a density of 1×10⁶ cells/well and transfected with 2 ug of pmiRGlo plasmid (Promega) using Lipofectamine 2000 (Life Technologies). After 24 hours, cells were re-seeded into 96-well plates containing Fol-DBCO-siLuc2, Fol-nigericin-DBCO-siLuc2, Fol-DBCO-NC (negative control), Fol-nigericin-DBCO-NC in folic acid and serum free RPMI medium for a final concentration of 50 nM. Untreated and unconjugated duplex miRNA were also included as controls. For each time point, renilla and firefly luciferase values were obtained using the Dual Luciferase Reporter kit (Promega) following the manufacturer's instructions. Firefly/Renilla ratios were normalized to Fol-DBCO-NC or Fol-nigericin-DBCO-NC for each time point. Experiments were performed three times with technical triplicates for each condition. Two-way analysis of variance (ANOVA) and Bonferroni post hoc test were used to test for statistical significance.

siRNA Gene Knockdown Assay Results and Discussion

Luciferase targeting assays were performed whereby MDA-MB-231 cells were incubated with a siRNA for luciferase (siLuc2) conjugated to folate (Fol-DBCO-siLuc) or a modified folate ligand carrying a molecule of nigericin (Fol-nigericin-DBCO-siLuc). These cell based experiments indicated a rapid reduction in luciferase activity in Fol-nigericn-DBCO-siLuc treated cells as soon as 18 hours post treatment and reaches 40% after 24 h (FIG. 2). However, there is no reduction in luciferase activity in Fol-DBCO-siLuc even up to 32 h. Interestingly, this reduction in luciferase activity was not observed on Fol-NC (negative control) treated cells or in Fol-siLuc treated cells suggesting that it is specific to the presence of nigericin.

The difference in luciferase repression between the Fol-siLuc2 and its nigericin counterpart demonstrates the facilitation of nigericin in the escape of folate-siLuc2 conjugate from the endosome to the cytoplasm.

Endosomal Escape Assay: Live cell Confocal Experiments

Live Cell Imaging

For live cell imaging, cells were plated on two-well chambered slides with glass bottom (Lab-Tek™ Chambered Coverglass, Thermo Fisher Scientific, Denmark). Briefly, chambered slides were pre-treated with Poly-D-Lysine (0.1 mg/mL; Sigma-Aldrich) for five minutes, washed with PBS and let to air dry for five minutes. MDA-MB-231 cells stably expressing Rab5B-GFP were plated one day before the experiment at 3×10⁴ cells/well and maintained in RPMI 1640 medium, no folic acid (Life Technologies) supplemented with 10% fetal bovine serum (Sigma), 100 U ml⁻¹ penicillin and 100 μg ml⁻¹ streptomycin (Hyclone, GE Healthcare Life Sciences) at 37° C. in 5% CO2. On the day of the experiment, and medium was replaced with medium supplemented with 10 mM 4-(2-hydroxyethyl)-1-piperazineethanesulfonic acid (HEPES, Sigma-Aldrich) and slides were placed in a Nikon A1Rsi confocal microscope with a resonant scanner and piezo z-drive (Nikon Instruments Inc.) equipped with a Tokai hit live imaging chamber (INU-TIZ-F1; Tokai Hit Corp., Japan) with temperature set to 37° C. Image acquisition was conducted on a single focal plane and was started after addition of folate-Cy5 conjugates (50 nM) using NIS-elements software 4.5 (Nikon Instruments Inc., Japan). Images were further analyzed using ImageJ 2.0.0 (NIH).

Live Cell Imaging

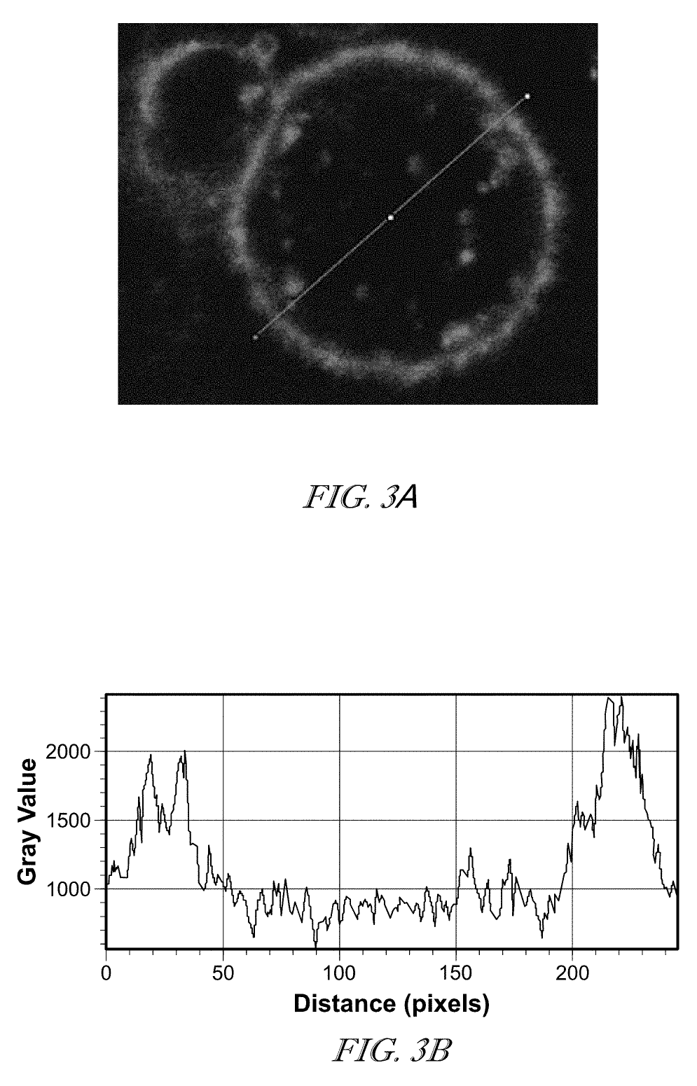

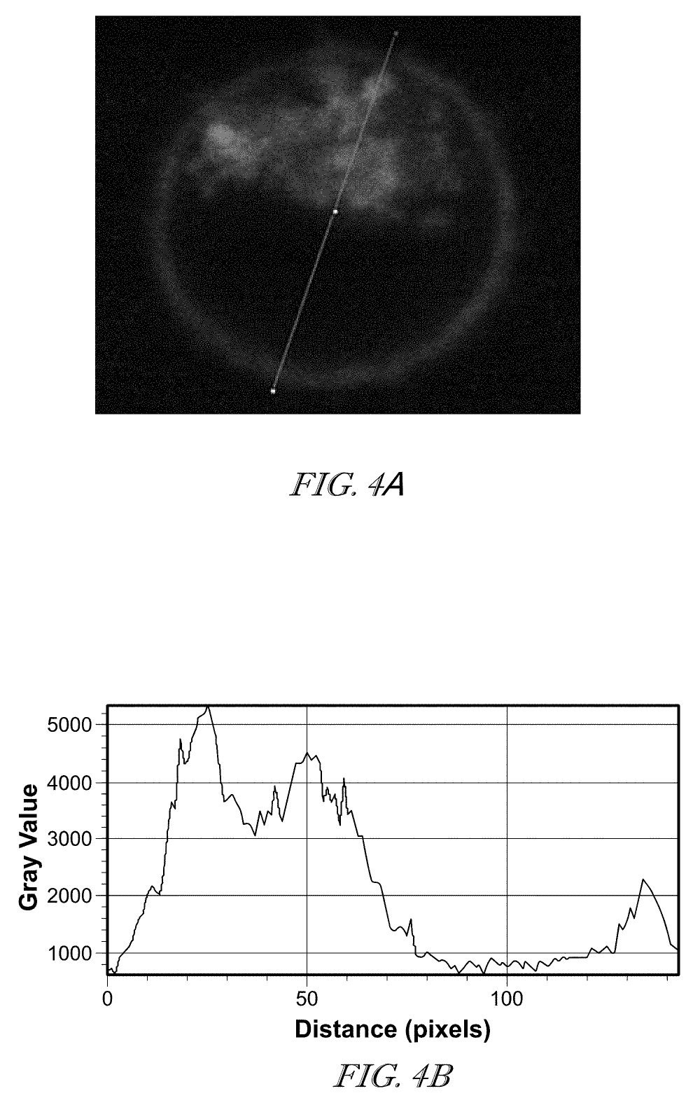

Initial endosomal escape experiments were performed in MDA-MB-231 cells stably expressing Rab5B-GFP using 50 nM of the folate-cy5 dye conjugate or folate-nigericin-Cy5 dye conjugate. Observations were made hourly throughout periods up to 24 h. Representative images of the cells at 3 h are presented in FIGS. 3 and 4. A significant difference in the fate of the folate-cy5 conjugate was apparent within 3 h after internalization when noticeably larger endosomes were observed in the cells treated with folate-nigericin-cy5 dye conjugate. After 3 h, the endosomes treated with folate-nigericin-cy5 were larger than their folate-cy5 dye conjugate and had begun to aggregate and plume. After 3 h, the swollen endosomes had aggregated into larger structures, whereas those treated only with folate-cy5 dye 3, remained relatively unchanged throughout the course of the experiment. FIG. 3 shows that Folate-Cy5 treated MDA-MB-231 cells gives Cy5 fluorescent signal mainly from cell membrane and endosome as punctate in 3 h post treatment. However, in Folate-nigericin-Cy5 treated cells the formation of large endosomes and cloudy dispersions of the fluorescent signal of Cy5 in the cytoplasm (FIG. 4).

### Experimental Design:

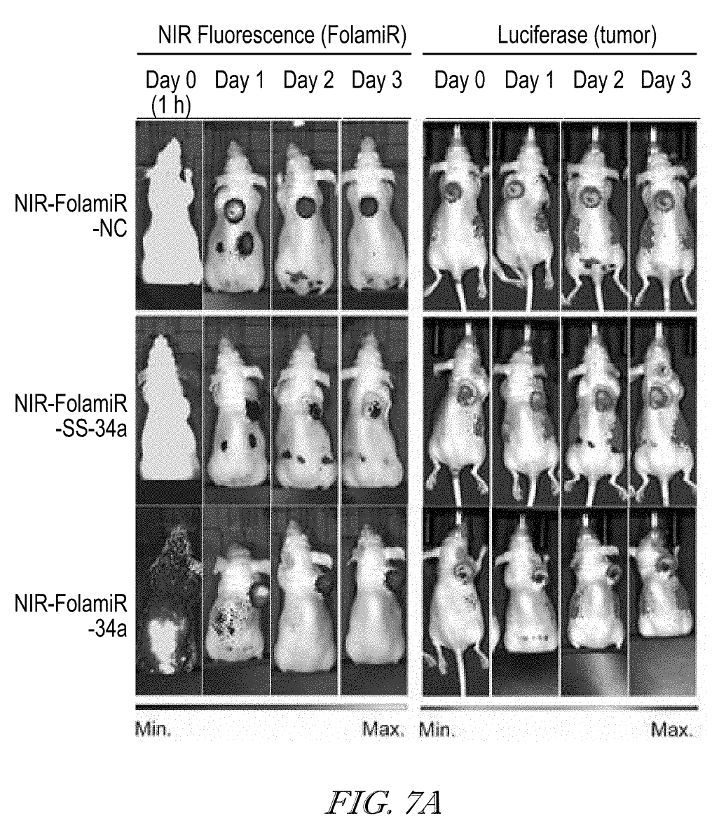

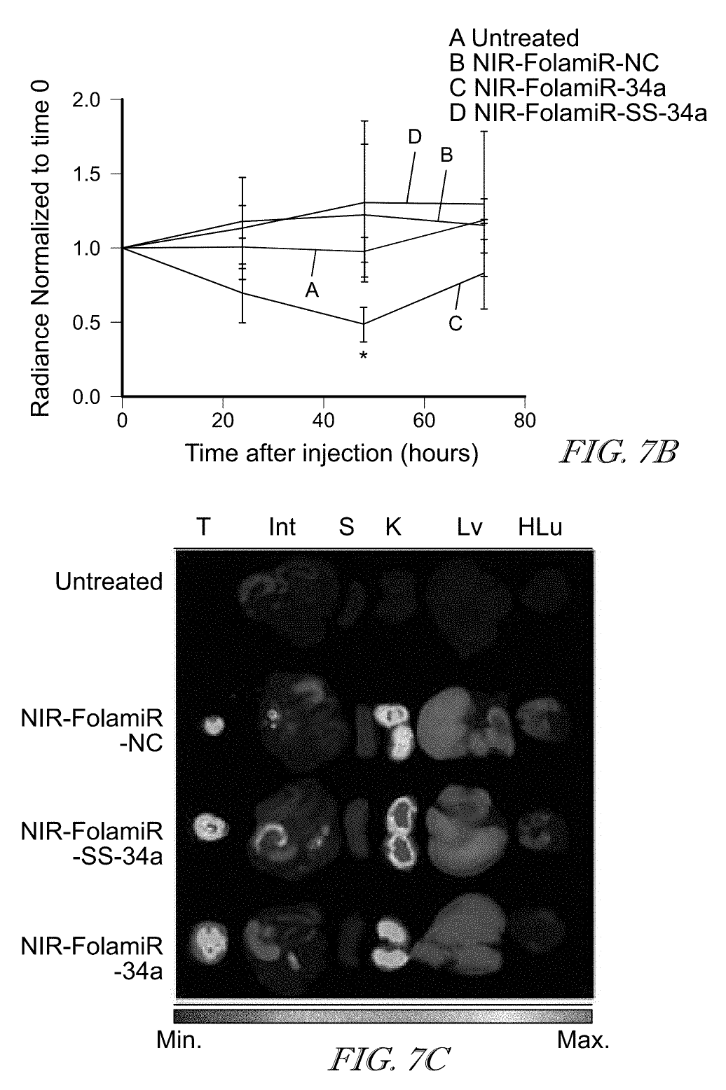

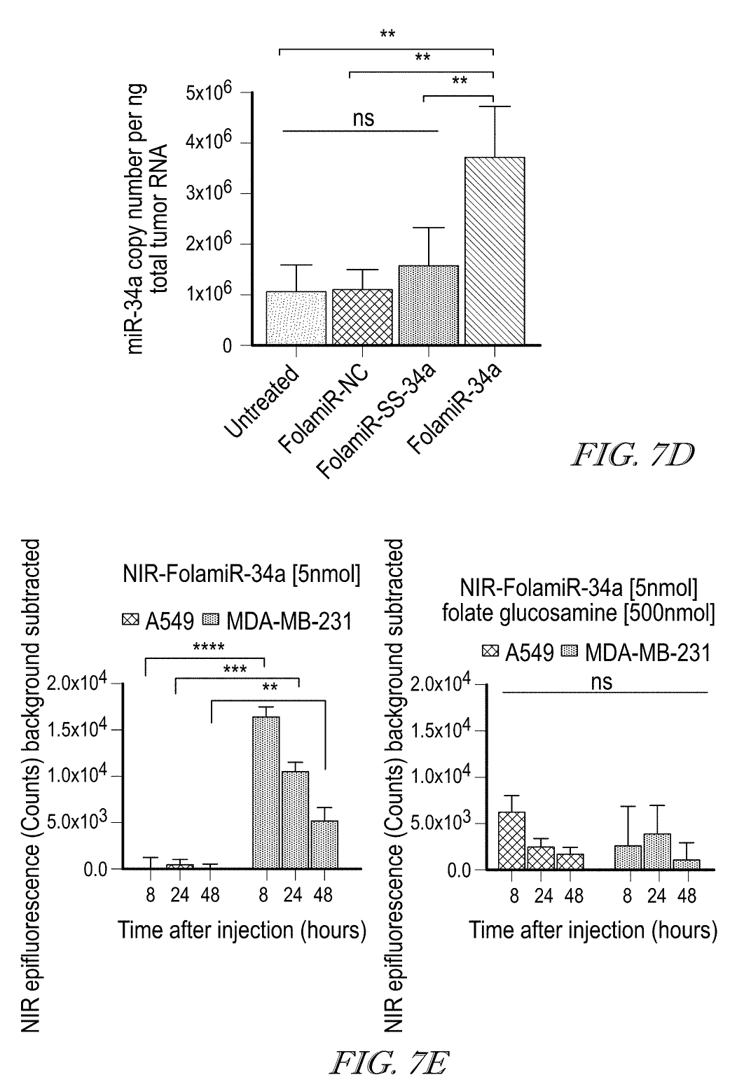

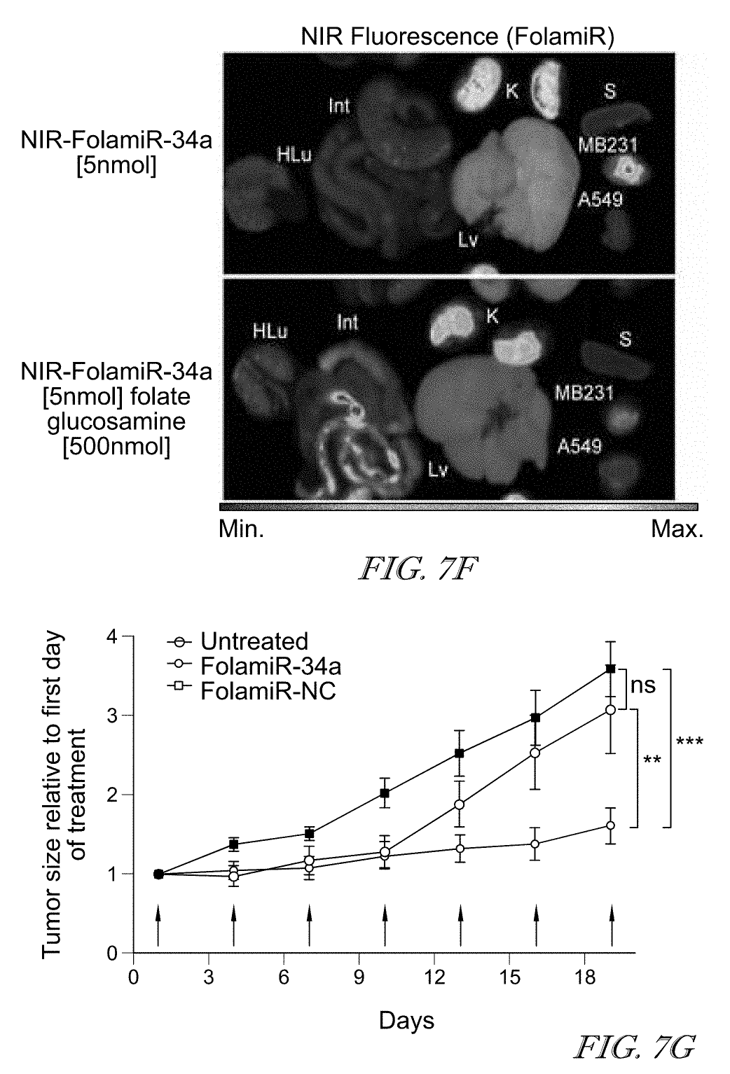

A priori power analysis was used to estimate sample size requiring a statistical significance of 0.05, alpha <0.5, and 80% power. Based on the power calculation the suggested number of animals to include in each treatment group was six. The expected effect size was then determined from small pilot studies. Due to a strong and significant reduction in renilla reporter activity observed after treating three animals with FolamiR-34a (FIGS. 7A, B), the remaining three animals were not treated. In this case the power of 0.5 accurately predicts significance using three animals with an alpha of <0.5. For all other studies, the initially calculated six animals were used.

### Flow Cytometry

FR positive human MDA-MB-231 cells and FR negative human A549 cells grown as described previously were detached by trypsinization and washed twice in ice-cold phosphate buffered saline (PBS; pH 7.4) and resuspended to a density of 1×10⁷ cells/mL in serum free medium. Cell viability was determined by trypan blue exclusion and cells were only used if the viability of cells was >80%. Next, flow cytometric analyses were performed following stanadard protocols. Briefly, 1×10⁶ cells were incubated with PE anti-FOLR1 antibody (Cat. 908303, Biolegend) or matched isotype antibody (Cat. 400213, Biolegend) as a control and analyzed by flow cytometric analysis using LSR Fortessa flow cytometer (BD Biosciences, San Jose, Calif., USA). Data was analyzed using FlowJo software v10 (Tree Star, Inc, OR, USA). Functionality of the FR was confirmed firstly by incubating MDA-MB-231 and A549 cells with FolamiR-34a-NIR (50 nM) followed by flow cytometric analyses as described above, and secondly by microscopy analisis of cells in incubated with folate-fluorescein isothiocyanate (FITC) to a final concentration of 50 nM. Cells were evaluated at different time points using an Olympus IX73 microscope equipped with a 1.25× objective, Olympus DP80 camera, and CellSens 1.11.

### In Vitro FolamiR Delivery

MDA-MB-231 triple-negative breast cancer cells (HTB-26) and A549 non-small cell lung cancer cells (CCL-185), both mycoplasma free as determined by testing for mycoplasma contamination via MycoAlert Mycoplasma Detection Kit (Lonza), were grown in RPMI 1640 medium, no folic acid (Life Technologies) supplemented with 10% fetal bovine serum (Sigma), 100 U mL⁻¹ penicillin and 100 μg mL⁻¹ streptomycin (Hyclone, GE Healthcare Life Sciences) and maintained at 37° C. in 5% CO2.

MDA-MB-231 and A549 cells were transfected with 500 ng of miR-34a Renilla sensor and incubated with FolamiRs. Renilla signal was measured 96 hours post treatment. Data points were normalized to FolamiR-NC (negative control: scrambled miRNA) for each time point. Error bars represent mean±s.d. Each experiment corresponds to n=3 with at least 4 technical replicates per treatment, statistical analysis performed with a one-way ANOVA with post hoc Bonferroni correction, (**, P<0.01).

For luciferase reporter experiments, a miR-34a sensor plasmid was generated by inserting the antisense sequence to miR-34a into the 3′ untranslated region of Renilla luciferase in the vector (psiCHECK, Promega). MiR-34a specific silencing was confirmed in MDA-MB-231 cells by transiently transfecting a miR-34a sensor or a mutated miR-34a sensor. MiR-34a sensor expressing cells were transfected with a miR-34a mimic using Lipofectamine RNAimax (Life Technologies) to confirm silencing mediated by exogenous miRNA. To generate stable clones, MDA-MB-231 cells were seeded in six-well plates at a density of 1×10⁶ cells/well and were transfected with 2 μg of miR-34a sensor plasmid using Lipofectamine 2000 (Life Technologies). Stable clones were selected using Hygromycin B (500 μg/mL; Hyclone, GE Healthcare Life Sciences) as a selection marker. Single clones were evaluated for Renilla expression and the clone with the highest Renilla expression was selected.

MB-231 sensor cells were seeded into 96-well plates containing FolamiR-34a, Fol-SS-34a and FolamiR-NC (negative control) in folic acid and serum free RPMI medium for a final concentration of 50 nM. Untreated and unconjugated duplex miRNA were included as controls. Renilla luciferase values were obtained 24, 48, 72, 96 and 120 h post incubation using the Renilla Glo Luciferase kit (Promega) following the manufacturer instructions. Renilla levels were normalized to FolamiR-NC for each time point. Experiments were performed three times with technical triplicates for each condition.

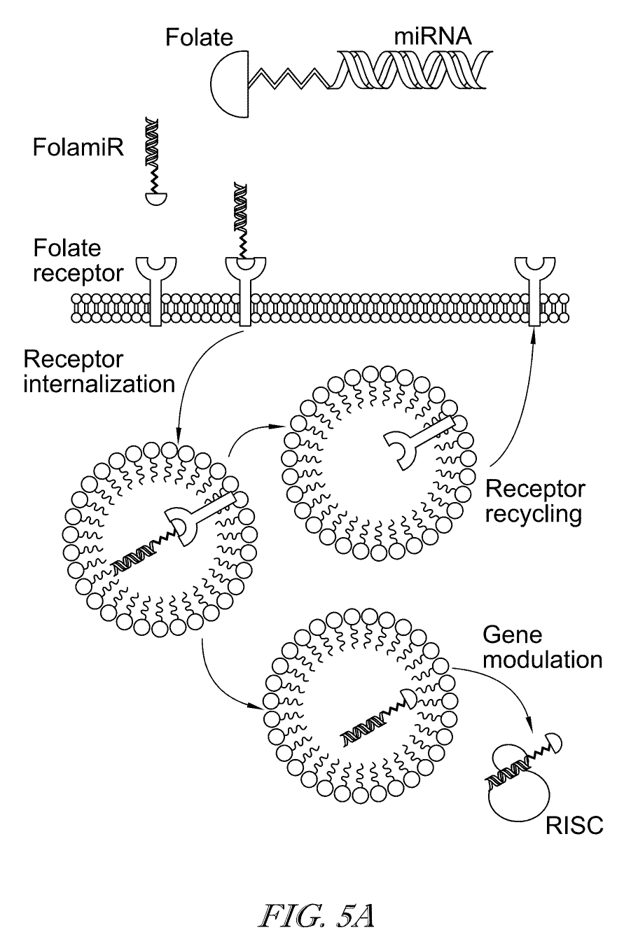

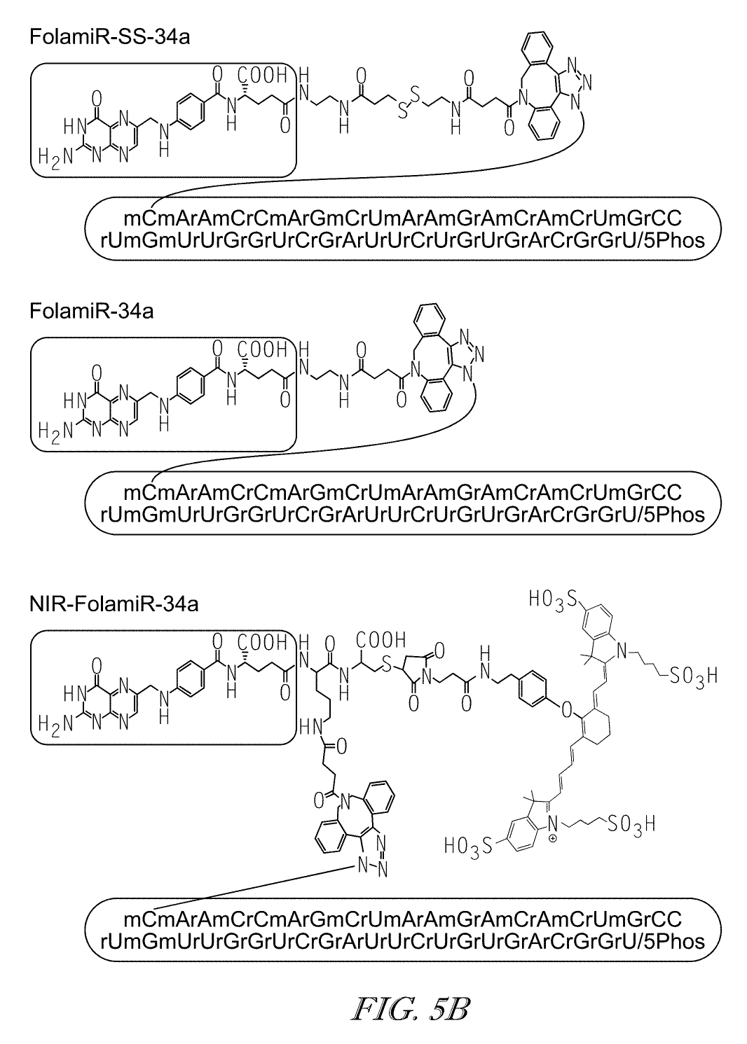

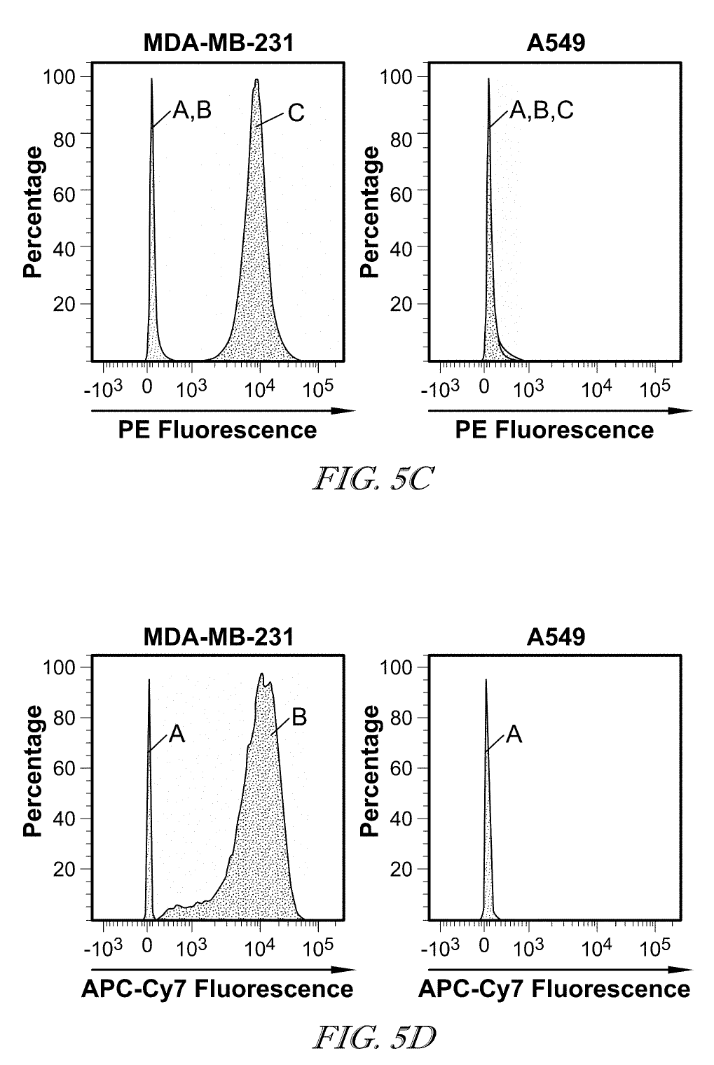

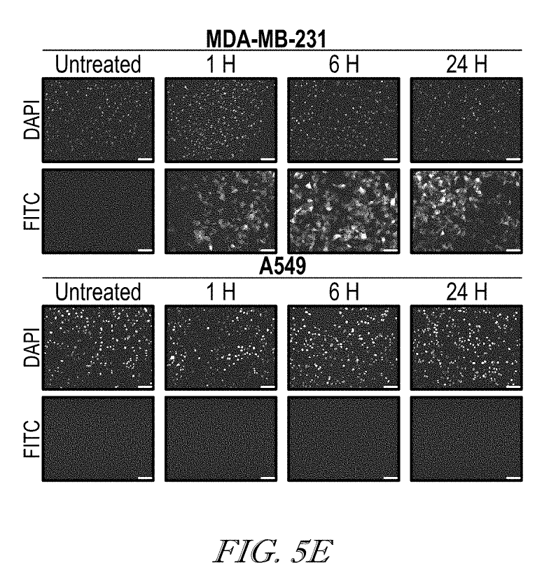

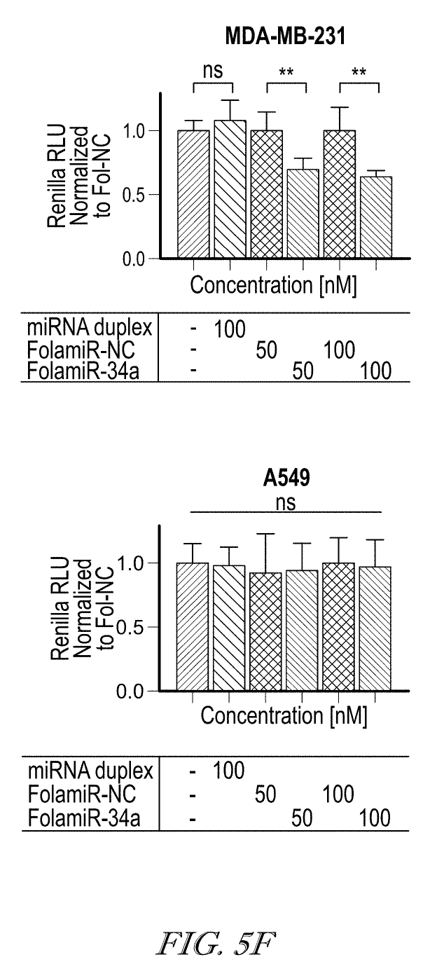

MDA-MB-231 breast cancer cells have been reported to express detectable levels of FR on the plasma membrane making this cell line a plausible model for evaluating FolamiR activity. To verify expression of the FR flow cytometric analyses was performed comparing MDA-MB-231 cells and A549 cells (FR negative control). MDA-MB-231 cells were confirmed to express detectable levels of FRα (FIG. 5c).

These results were corroborated by analyzing the cellular uptake of NIR-FolamiR-34a using flow cytometry (FIG. 5d) and folate-fluorescein isothiocyanate (Fol-FITC) conjugate uptake using fluorescent microscopy (FIG. 5E). Both folate conjugates were taken up by the FR positive cell line MDA-MB-231 but not by the FR negative A549 cell line. This observation was functionally confirmed following treatment of MDA-MB-231 and A549 cell lines transiently expressing a miR-34a Renilla sensor with FolamiRs. The sensor is a Renilla gene followed by a single miR-34a complementary binding site, allowing for monitoring of the post-transcriptional regulation of Renilla by miR-34a. The sensor in both cell lines was responsive to transfected miR-34a mimics (FIG. 10); however, the sensor was only downregulated in MDA-MB 231 cells following FolamiR-34a exposure (FIG. 5F), suggesting that FolamiR targeting is dependent on FR expressing cells. Taken together these results suggest that MDA-MB-231 is a FR positive cell line and provide preliminary evidence for the specific uptake of FolamiR conjugates via FR interaction.

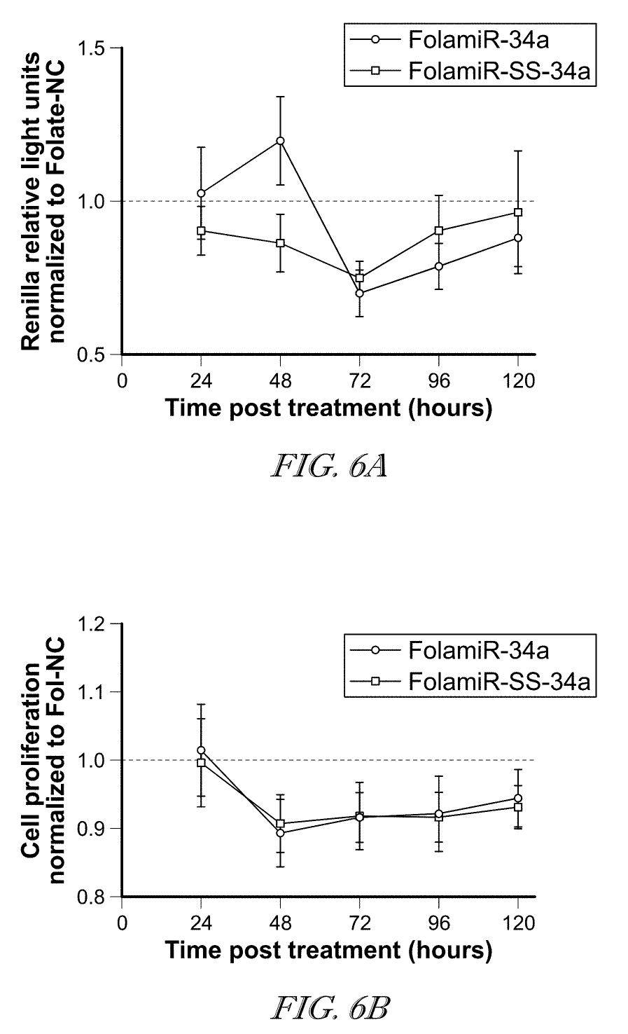

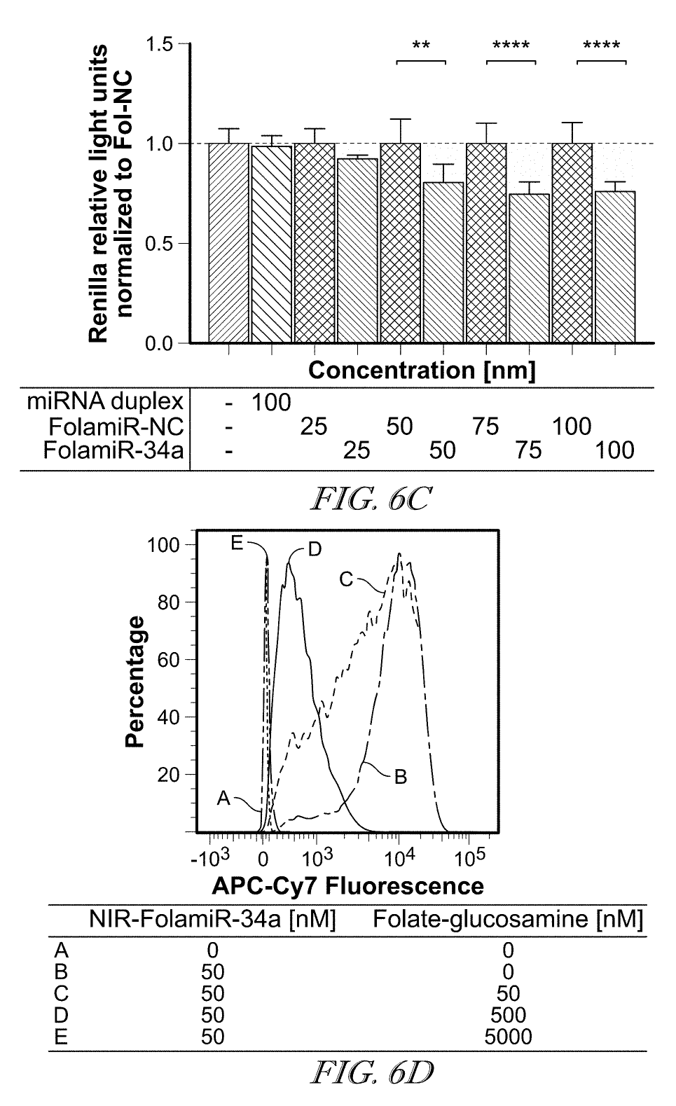

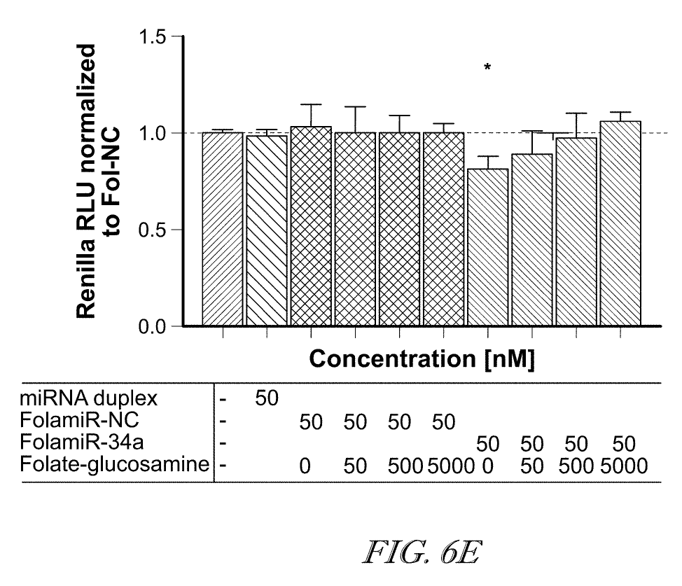

To monitor FolamiR-34a activity, MDA-MB-231 cells were generated to stably express the miR-34a Renilla luciferase sensor (MB-231 sensor) or a mutated version of the sensor that is unresponsive to miR-34a. Multiple clones were generated and the clone with the highest level of Renilla (FIG. 11) was used to assess FolamiR-34a activity. When FolamiR-34a or FolamiR-SS-34a was added to the MB-231 sensor cells (in the absence of transfection reagent) there was a decrease in Renilla activity 72 hours after exposure (FIG. 6a). Renilla activity rebounded 120 hours following exposure, likely due to replication-induced dilution of FolamiR-34a in the cells or degradation of FolamiR-34a.

Proliferation of MB-231 sensor cells was reduced following a single FolamiR-34a treatment (FIG. 6B), which correlated with the reduction in Renilla activity. Surprisingly, both the releasable and unreleasable FolamiRs efficiently entered the cell and retained activity based on the data, suggesting that the conjugated folate does not interfere with loading of miR-34a-5p into Argonaute.

### FR Dependent Response

FR positive human MDA-MB-231 cells and FR negative human A549 cells were transfected with 500 ng of a miR-34a sensor plasmid using Lipofectamine 2000 (Life Technologies). After 24 hours 4000 cells/well were seeded into 96-well plates containing FolamiR-34a and FolamiR-NC (negative control) in folic acid and serum free RPMI medium for a final concentration of 50 nM or 100 nM. Cells transfected with Lipofectamine RNAimax (Life Technologies) were used as a control to monitor miR-34a Renilla sensor response to miR-34a mimic. Untreated and unconjugated duplex miRNA were included as controls. Renilla luciferase values were obtained 96 hours post incubation using the Renilla Glo Luciferase kit (Promega) following the manufacturer instructions. Renilla levels of FolamiR-34a treated cells were normalized to FolamiR-NC for each time point and unconjugated duplex miRNA treated cells were normalized to untreated. Experiments were performed three times with technical triplicates for each condition. A dose-dependent reduction in Renilla activity was only observed in cells treated with FolamiR-34a (FIG. 6c).

### In Vitro FR Binding Competition Assay

In vitro FR binding competition assays were performed as described previously in Van Der Heijden, J. W. et al. (“Folate receptor β as a potential delivery route for novel folate antagonists to macrophages in the synovial tissue of rheumatoid arthritis patients,” Arthritis Rheum. 60, 12-21 (2009)) and Gent, Y. Y. et al. (“Evaluation of the novel folate receptor ligand [18F]fluoro-PEG-folate formacrophage targeting in a rat model of arthritis,” Arthritis Res. Ther. 15, R37 (2013)). Briefly, FR positive human MDA-MB-231 cells and FR negative human A549 cells grown as described previously (were detached by trypsinization and washed twice in ice-cold phosphate buffered saline (PBS; pH 7.4) and resuspended to a density of 1×10⁷ cells/mL in serum free medium. Next, 100 μL of this cell suspension was incubated with FolamiR-34a-NIR to a final concentration of 50 nM in the absence or presence of 1 to 100 fold molar excess of folate glucosamine conjugate. Cells were incubated at 4° C. for 20 minutes and washed twice with ice cold PBS and analyzed for displacement of FolamiR-34a-NIR binding by flow cytometric analysis using LSR Fortessa flow cytometer (BD Biosciences, San Jose, Calif., USA). Data was analyzed using FlowJo software v10 (Tree Star, Inc, Ashland, Ore).

Functional competition was verified using MDA-MB-231 sensor cells incubated with FolamiR-34a or FolamiR-NC to a final concentration of 50 nM in the absence or presence of 1 to 100 fold molar excess of folate glucosamine conjugate. Untreated and unconjugated duplex miRNA were included as controls. Renilla luciferase values were obtained 96 hours post incubation using the Renilla Glo Luciferase kit (Promega) following the manufacturer instructions. Renilla levels of FolamiR-34a treated cells were normalized to FolamiR-NC for each time point and unconjugated duplex miRNA treated cells were normalized to untreated. Experiments were performed three times with technical triplicates for each condition.

Increasing the amount of folic acid glucosamine conjugate (folate-glucosamine) resulted in a dose-dependent reduction in cell-specific NIR-FolamiR-34a signal, indicating that folate-glucosamine competes with NIR-FolamiR-34a (FIG. 6D). More importantly, folate-glucosamine treatment abrogated the silencing effect of NIR-FolamiR-34a on the miR-34a Renilla sensor in a dose-dependent manner (FIG. 6E). These results support that FolamiRs can deliver functional miRNAs to cells overexpressing the FR and that the delivery is dependent on FR expression.

FIGS. 6D, E show miR-34a Renilla sensor response to FolamiR-34a (50 nM, 96 h) in presence of increasing concentrations of folate glucosamine conjugate. Data points were normalized to FolamiR-NC (negative control: scrambled miRNA) for each experimental condition. Experiment corresponds to n=3 with at least 4 technical replicates per treatment, statistical analysis performed with a one-way ANOVA with post hoc Bonferroni correction (*P<0.05).

### Cell Proliferation Assays

A Sulforhodamine B (SRB, Sigma) assay was used as a proxy for cell proliferation in 96-well plates. Briefly, following FolamiR treatment cells were fixed with 10% tricholoroacetic acid in complete media and stained for 1 hour with 0.4% (wt/vol) SRB in 1% acetic acid. Unbound dye was removed by four washes with 1% acetic acid. Finally, protein-bound dye was extracted with 10 mm unbuffered Tris base and absorbance at 510 nm was obtained using a GloMax Multi+spectrophotometer (Promega). Absorbance values (proxy for cell mass) were normalized to that of cells cultured in the presence of FolamiR-NC for each time point.

### Flank Tumor Establishment

For single-dose studies, subcutaneous tumors were induced in female Nu/Nu (NU-Foxn1nu; Charles River) congenic mice (6 weeks, n=5) by subcutaneous injection of 5×10⁶ MDA-MB-231 sensor cells suspended in 200 μL of Matrigel (Corning). For longitudinal studies, parental MDA-MB-231 cells were used. Due to the observation that rodents present high plasma and tissue levels of 5-methyl-tetrahydrofolate, the naturally occurring form of folate, (around 10-fold higher than in humans) mice were maintained on folic acid deficient diet (Envigo, TD.95247) two weeks prior tumor implantation and during the experiment series. A folate-deficient diet has shown to reduce folate levels to physiological levels seen in humans. To determine tumor growth, individual tumors were measured using a vernier caliper and tumor volume was calculated by: tumor volume (mm³)=width×(length²)×2⁻¹. Animals were excluded if tumors had not reached a volume of 150 cm³ by the time of treatment. For single dose experiments, animals were injected intravenously (i.v.) with 5 nmol of FolamiRs after acquisition of luminescent and fluorescence signals (day 0). For multiple dosing experiments, animals were randomized into experimental arms by minimizing the differences in their mean tumor size.

When tumor volume reached ˜200 mm³ animals were treated with i.v. injections of the indicated molar concentration of FolamiR every three days. All experimental protocols were approved by the Purdue Animal Care and Use Committee and were in compliance with NIH guidelines for animal use.

### Bioluminescent and Infrared Imaging

RediJect Coelenterazine h Bioluminescent Substrate (PerkinElmer) was administered per the manufacturer's protocol for in vivo monitoring of tumor bioluminescence using IVIS Lumina II (Caliper) or Spectral AMI (Spectral Instruments). Luminescent values were acquired at multiple points after injection of substrate starting at 20 minutes and only maximal mean radiance values were reported. Infrared imaging was conducted using IVIS Lumina II (Caliper) at 745 nm excitation and ICG emission filters. Non-invasive longitudinal monitoring of tumor luminescence and fluorescence was conducted by whole-animal imaging performed at the following time points: 0, 24, 48, and 72 h (n=3 animals per experimental group). Gross organ images were acquired using the 800 nm channel in the Licor Odyssey CLX (Licor).

### Serum Cytokines

Serum samples from multiple dosing experiments (24 hours after last injection) were used to test for IL-6, and tumor necrosis factor (TNFα) concentrations using the mouse specific cytokine Multi-Analyte ELISArray Kit (Qiagen) according to manufacturer's instructions. Briefly, serum samples were thawed on ice and cleared from debris by centrifugation at 1000×g at 4° C. for 10 min before the analysis. All samples or standards were added to a 96-well plate together with assay buffer. Plates were shaken gently and incubated for 2 h at room temperature.

Supernatant was removed and wells were washed. A detection antibody was added and the plates were incubated for 1 h at room temperature. Plates were rinsed and incubated with Avidin-HRP solution for 30 min at room temperature. The wells were washed and development solution was added to acquire data using a GloMax plate reader (Promega). Absorbance values were acquired at 450 nm and 570 nm. The 570 nm readings were subtracted from the 450 nm readings. Cytokine standard curves were used to calculate the cytokine concentrations in serum samples (pg/ml). The limits of detection were as follows: IL-6 58.8 pg/mL, and TNFα 30.5 pg/mL. LPS treated Nu/Nu mice (NU-Foxn1nu; Charles River) were used as positive controls. Mice received an intra-peritoneal injection of 500 ng kg⁻¹ lipopolysaccharides (LPS, L6529, Sigma) and serum was collected two hours post injection.

Maximum Tolerated Dose (MTD) study

Balb/c mice (8 weeks of age) were administered one intravenous injection of 33.3, 10 or 1 nmol of FolamiR-34a. Animals were observed post administration for 2 weeks. The mice were observed for changes in body weight and clinical observations (rapid weight loss, diarrhea, rough hair coat, hunched posture, lethargy, labor breathing, neurological signs, etc.). The mice were allowed ad libitum feed and water. A necropsy was performed at the end of the study. Whole blood, serum, and organ tissue were collected for further analysis.

### Activity of FolamiRs In Vivo, in a Xenograft Model of Breast Cancer.

A single 5 nmol dose of each NIR-Fol tagged miRNA (NIR-FolamiR) was delivered via tail vein injection into animals with palpable MB-231 sensor cell xenografts. Fluorescent distribution and luciferase activity was measured to monitor for tumor cell targeting specificity, and as a surrogate for uptake and intercellular target repression, respectively.

Twenty-four hours after injection, NIR-FolamiR was primarily retained in tumor tissues, and importantly, cleared from the rest of the organism (FIG. 7A, left-NIR), including the liver (FIG. 7C, Lv). However, only the unreleasable NIR-FolamiR-34a induced Renilla knockdown in vivo (FIG. 7A, right-luciferase, quantified in FIG. 7B). Importantly, after only a single injection, Renilla levels were reduced approximately 50% following NIR-FolamiR-34a treatment, which was even greater than the reduction in sensor activity observed in cells in culture (FIGS. 6A, C, E). Approximately 3.5×10⁶ copies of miR-34a per nanogram of total RNA were present in the tumors treated with NIR-FolamiR-34a (FIG. 7D).

In contrast, the copy number of miR-34a in the tumors harvested from mice treated with NIR-FolamiR-SS-34a was similar to the negative control animals, suggesting that the releasable folate-conjugate may be degraded or prematurely reduced in circulation. To address this possibility, FolamiR conjugates were exposed to 50% serum. FolamiR-SS-34a was highly unstable in the presence of serum while FolamiR-34a remained intact for more than six hours.

FolamiR-34a appeared more stable than unconjugated miR-34a, suggesting that folate protects the miRNA from serum nucleases. To determine if the FolamiRs bind specifically to FR in vivo, 5 nmol of NIR-FolamiR-34a was injected intravenously in the presence or absence of 100-fold molar excess of folate-glucosamine in nude mice bearing FR positive human MDA-MB-231 sensor cells engrafted on the right shoulder and FR negative human A549 cells engrafted on the left side shoulder. The results indicate that FR positive MDA-MB-231 tumors accumulate the FolamiR conjugate, but not FR negative A549 tumors (FIG. 7E, F) and that this FR dependent accumulation can be blocked by an excess of folate-glucosamine (FIG. 7E, F).

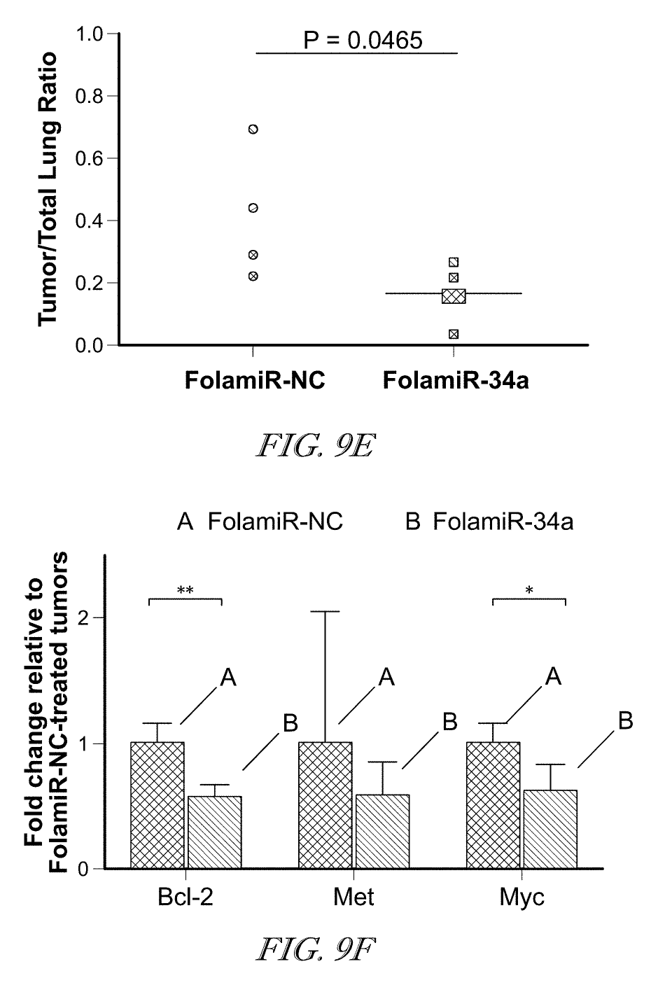

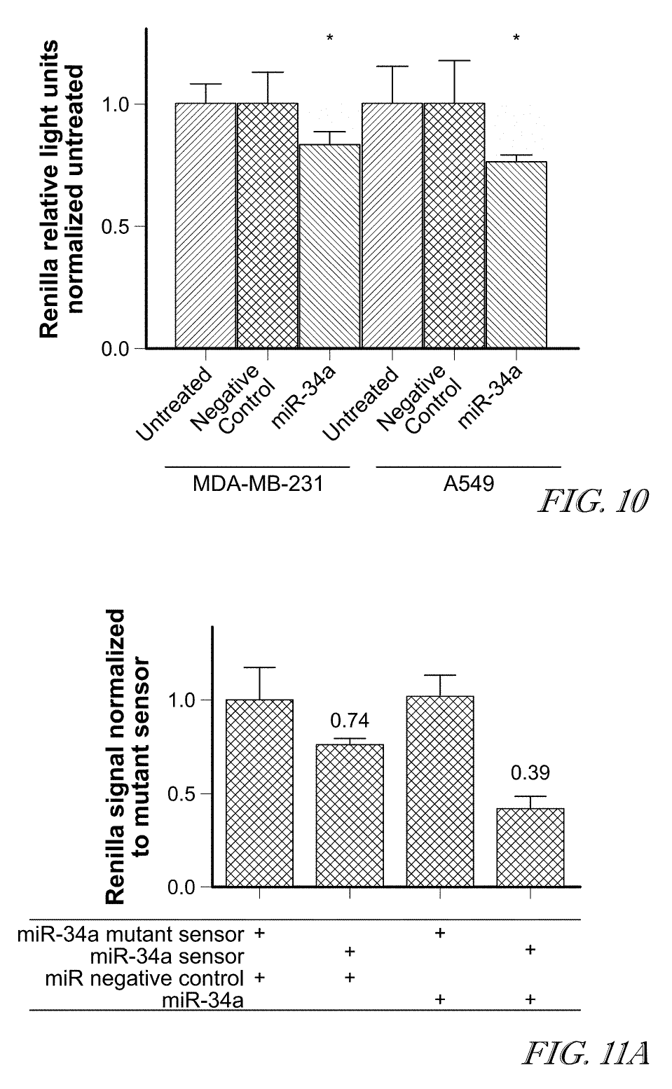

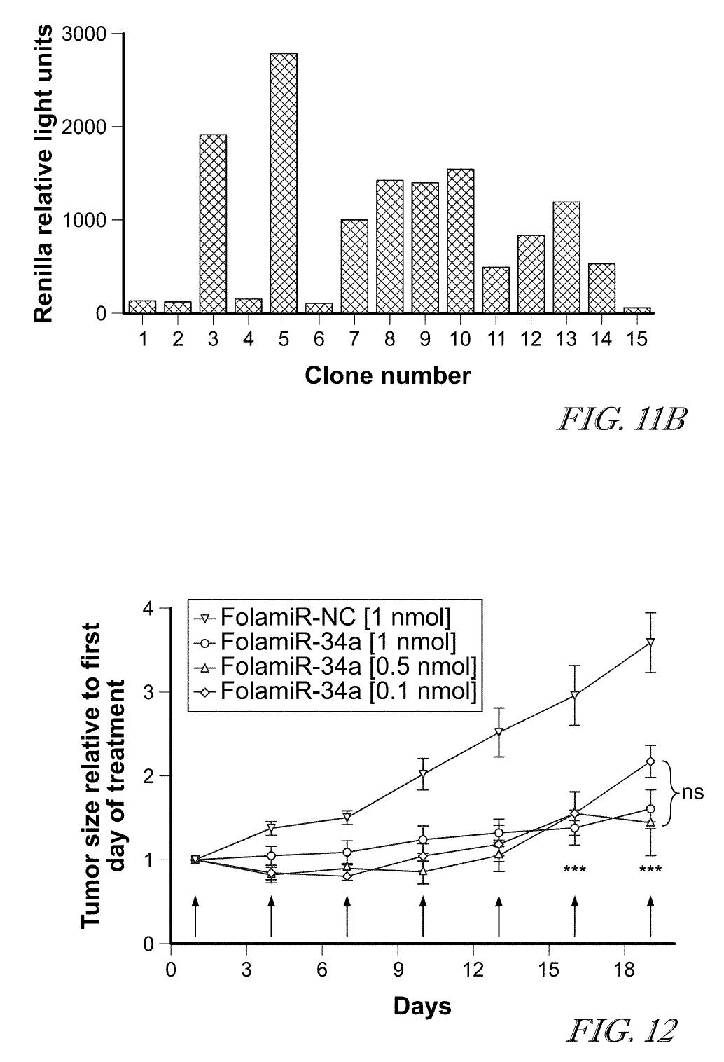

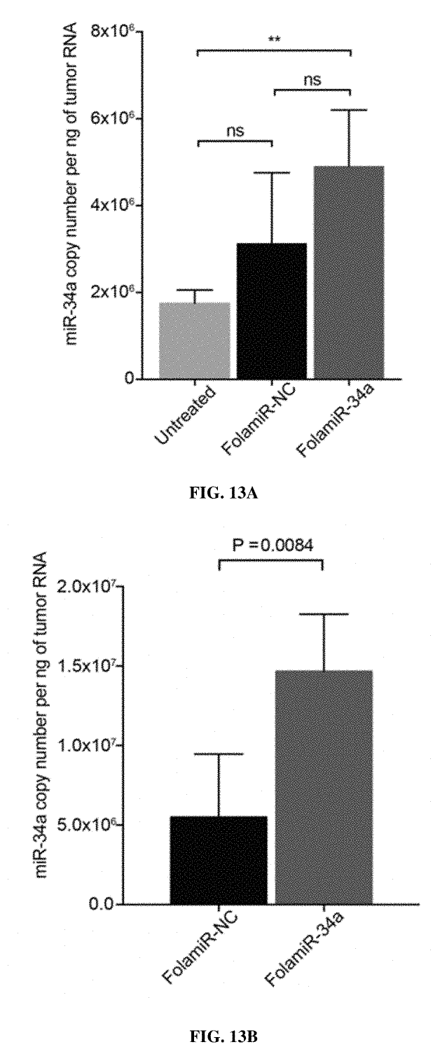

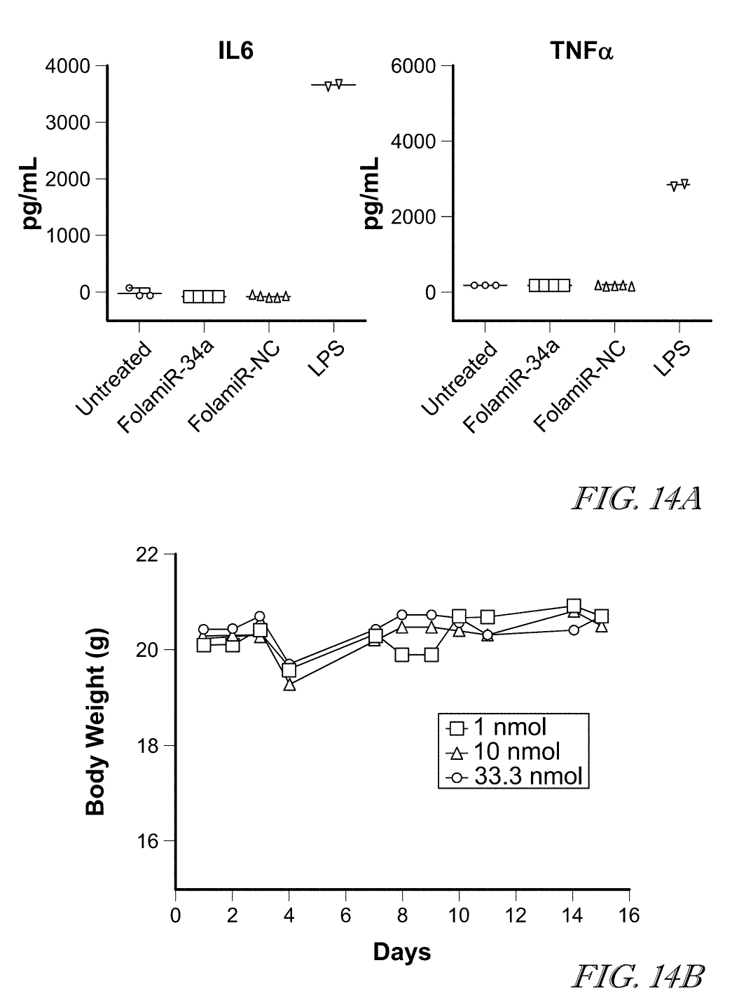

Next, a multiple-dosing study was performed to evaluate the efficacy of FolamiR-34a. MB-231 xenograft animals were treated with reduced doses of FolamiR-NC or FolamiR-34a (0.1, 0.5 and 1 nmol) every three days for a total of seven doses. Tumors in animals administered the control folate-conjugate grew approximately 3.5-fold, while tumor size in animals treated with FolamiR-34a increased modestly (˜1.5 fold) during the 20-day dosing period (FIG. 7G and FIG. 12). Doses as low as 0.1 nmole produced a significant reduction in tumor growth. Copy number of miR-34a in the excised tumor tissue was approximately 1.5 fold higher than in the tumors extracted from mice administered the control (FIG. 13A). Importantly, there was no evidence of whole organ toxicity or elevation in the serum cytokines IL-6 or TNF-α (FIG. 14A) in animals treated with FolamiR-34a. These results are supported by a maximum tolerated dose (MTD) study performed in immunocompetent mice in which none of the mice dosed with FolamiR-34a presented with pathological signs of toxicity or significant changes in body weight up to the maximum dose tested of 33.3 nmol (FIG. 14B) indicating an MTD>33.3 nmol.

Induction of tumor formation in Kras;p53 mice

Induction of tumor formation in Kras^{LSL-G12D/+}; Trp53^{flx/flx} (FVB.129 background) double mutant mice (6 to 10 weeks old) was performed based on the method of DuPage, et al. (DuPage M, Dooley A L, Jacks T. Conditional mouse lung cancer models using adenoviral or lentiviral delivery of Cre recombinase. Nat. Protoc. 2009; 4:1064-1072). Briefly, lung specific transgene activation was achieved via intratracheal delivery of Adenoviral particles (10⁶ PFU) encoding for Cre recombinase. Tumors were allowed to preform for eight weeks prior experiments.

### Tumor Progression Monitoring Using Magnetic Resonance Imaging (MRI)

MRI scans of induced and non-induced animals (scans of healthy tissue) were obtained using a 7.0 Tesla Bruker Biospec 70/30 USR Scanner (Billerica, Mass.) and a 40 mm mouse volume coil at the Purdue MRI Facility. Animals were anesthetized using a 2.5% v/v isoflurane in O₂ for 5 minutes and then moved to the heated animal bed where anesthesia was set to 2%. Respiration rate was monitored via pressure sensor. A low-resolution multiplane scout scan was obtained using the following parameters: TR=4s, TE=1.5 ms, FOV=30×30 mm², slice thickness=1 mm, data matrix=256×256, 7 slices per plane (axial, coronal and sagittal), approximate time of scan per mice=1 minute. The scout scan was used to align the spine of the mouse to collect high-resolution images of the lungs using the following parameters: TR=4s, TE=1.5 ms, FOV=30×30 mm2, slice thickness=0.5 mm, data matrix=256×256, 30 slices per plane (axial and coronal), approximate time of scan per mice=5 minutes.

Quantification of tumor burden was conducted following the manual segmentation protocol described by Krupnick et al (“Quantitative monitoring of mouse lung tumors by magnetic resonance imaging,” Nat. Protoc., 2012, 7, 128-142). This analysis of tumor burden by MRI takes advantage of the vast difference in MR image intensities between tumor tissue (bright) and normal lung tissue (dark) and uses the average lung image intensity as a proxy for tumor burden. Briefly, lung MR images are manually segmented using ImageJ 2.0.0 and the average lung image intensity normalized to that of the liver within the same animal is calculated. To determine tumor progression within an animal the average lung image intensity is then normalized to the first day of treatment. Furthermore, tumor and whole lung volumes per animal were calculated using three-dimensional reconstruction using ITK-Snap and Paraview 5.2 software (Kitware, NY, USA). Tumor/whole lung ratios were obtained at the indicated times showing the percentage of lung volume occupied by tumors.

### Folate Uptake Studies in Kras;p53 Mice

For folate uptake studies, 5 nmoles of OTL38 (kindly provided by On Target Laboratories, LLC, West Lafayette, Ind.), a fluorescent imaging conjugate composed of folate tethered to a fluorescent near infrared (NIR) dye currently in clinical trials (Clinical trial identifier: NCT02769533) were delivered systemically through the tail vein into healthy and tumor bearing mice (n=3; 8 weeks post transgene activation). Twenty-four hours after the injection animals were sacrificed and perfused with saline. Whole organ images were acquired using the 800 nm channel in the Licor Odyssey CLX (Licor). Lungs were fixed in 10% buffered formalin and paraffin embedded according to standard procedures. Sections were stained by hematoxylin and eosin (H&E) and evaluated using an Olympus IX73 microscope equipped with a 1.25× objective, Olympus DP80 camera, and CellSens 1.11. Tumor burden was calculated using ImageJ 2.0.0, which represents the tumor area relative to the total lung area obtained from three independent sections for each animal. Unstained mounted sections were evaluated in the 800 nm channel in the Licor Odyssey CLX (Licor) and using an Nikon TiS microscope equipped with a 20× objective, an ICG band pass filter (Ex: 780-800; Ex: 810-860; Semrock, Brightline), a xenon/mercury light source (Nikon, Japan), Photometrics QuantEM EMCCD camera, and NIS-Elements (Nikon, Japan).

Since this model had not yet been validated for FR expression, firstly, pulmonary adenocarcinomas of this model were evaluated for folate receptor expression, and tumor-specific uptake and retention of folate conjugates. A fluorescent imaging ligand OTL38, folate receptor-alpha (FRα)-targeting ligand conjugated to a fluorescent near infrared (NIR) dye, was intravenously administered to mice bearing lung tumors or healthy individuals.

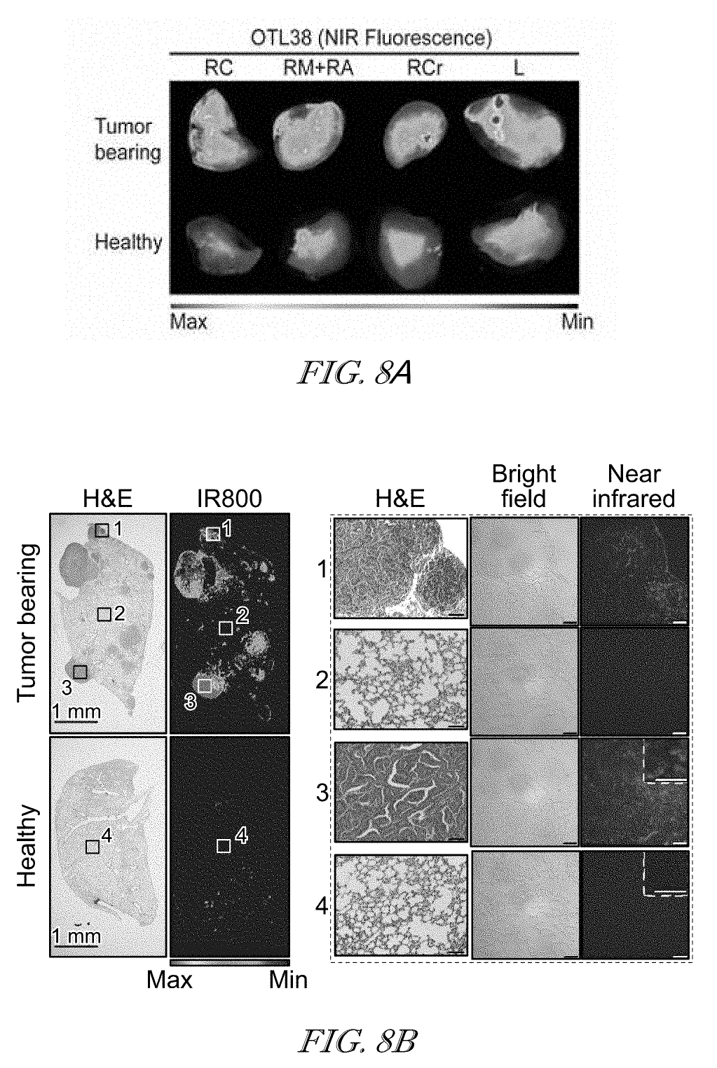

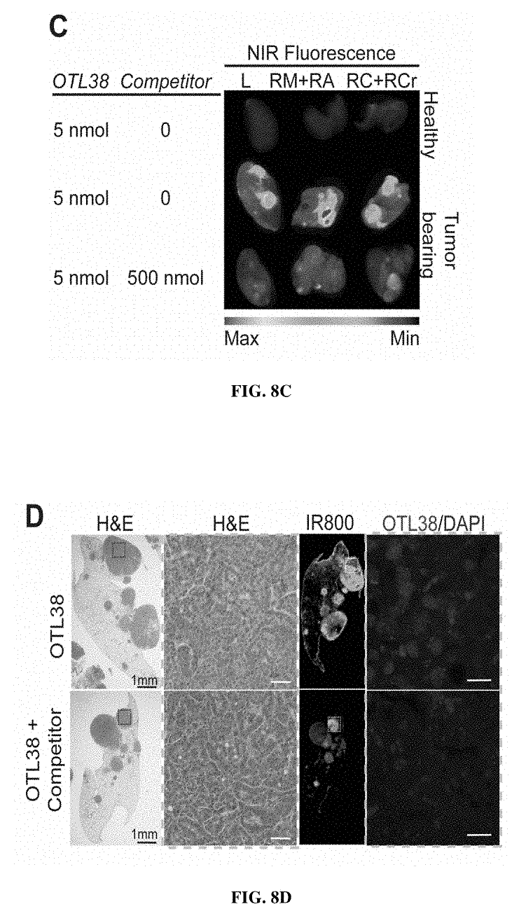

The folate conjugate was preferentially retained in lung tumors and cleared from normal healthy tissues as observed at the gross organ level (FIG. 8A) and at the histological level (FIG. 8B). Higher magnification images indicate that the near infrared signal is not an artifact of the cell density differences between healthy and malignant tissues; defined punctate signaling is observed in tumors following OTL38 administration, as has previously been observed due to receptor-mediated endocytosis of OTL-38 (see insets in FIG. 8B). To determine if OTL38 retention in pulmonary adenocarcinomas is mediated by its interaction with FR an in vivo blockade assay was performed. OTL38 (5 nmol) was injected intravenously in the presence or absence of 100-fold molar excess of folate-glucosamine in mice bearing lung tumors. OTL38 preferential retention in lung tumors was reduced by an excess of folate-glucosamine (FIGS. 8C, D) suggesting that OTL-38 accumulation in tumors is dependent on the FR. These data confirm that the KrasLSL-G12D/+; Trp53flx/flx tumors specifically take up and retain OTL-38, suggesting that FolamiR-34a should likewise accumulate in the tumor tissue.

### FolamiR Treatment in Kras;p53 Mice

For multiple dosing experiments with FolamiR-34a, tumor bearing animals (8 weeks) were randomized into experimental arms by minimizing the differences in their MRI measured tumor burden. Animals were treated with i.v. injections of 1 nmol FolamiR every three days (10 injections total) and tumor progression was monitored using a 7.0 Tesla Bruker Biospec 70/30 USR Scanner (Billerica, Mass.) as described above every week for four weeks. Twenty-four hours after the final injection animals were sacrificed and perfused with saline. Lungs were harvested, fixed in 10% buffered formalin and paraffin embedded according to standard procedures.

Sections were stained by hematoxylin and eosin (H&E) and evaluated as described earlier. Mice were maintained on a folic acid deficient diet (Envigo, TD.95247) starting at six weeks after tumor induction and during the experiment series. All experimental protocols were approved by the Purdue Animal Care and Use Committee and were in compliance with NIH guidelines for animal use.

### In Vivo Blocking of FR

Subcutaneous tumors were induced in female Nu/Nu (NU-Foxn1nu; Charles River) congenic mice (6 weeks, n=3) following injection of 5×10⁶ FR positive human MDA-MB-231 sensor cells (right side) and FR negative human A549 cells (left side) suspended in 200 μL of Matrigel (Corning). Tumors were allowed to form and mice bearing A549 and MDA-MB-231 tumors of similar size were included in the experiment. For the Kras;p53 mouse model, tumors were allowed to form for eight weeks after transgene activation (n=3). Tumor formation was monitored using MRI. Competition studies were performed in mice (n=3) by co-administration, via the tail vein, of 5 nmoles of FolamiR-34a-NIR for the xenograft model or 5 nmoles of OTL38 for the Kras;p53 mouse model in the presence or absence of 500 nmoles (≥100-fold molar excess) of folic acid glucosamine. Folic acid glucosamine conjugate was used because of its increased solubility at low pH compared folic acid and to prevent precipitation in the kidneys. In vivo whole animal imaging and ex vivo tissue distribution studies were performed as described above.

RNA Isolation and miRNA Expression Analyses Using Quantitative PCR (qPCR)

MDA-MB-231 derived tumors from Nu/Nu mice (NU-Foxn1nu; Charles River) and lung tumors from Kras^{LSL-G12D/+}; Trp53flx/flx mice were collected in RNA Later (Life Technologies) and stored at −80° C. Tumor tissues (50 mg) were placed in 2 mL collection tubes containing 700 μL QIAzol lysis reagent (Qiagen) and 1.4 mm ceramic beads. Samples were disrupted using a bead mill (Fisher Scientific) at 4 m s⁻¹ for 3 minutes. Total RNA was extracted using RNeasy Mini Kit (Qiagen) according to the manufacturer instructions. Next, cDNA was generated using miScript II RT Kit (Qiagen) and miScript HiFlex Buffer using 1 μg of total RNA. For miR-34a standard generation, miR-34a mimic (Life Technologies) was used for cDNA synthesis. qRT-PCR was performed with miRNA primer assays (Qiagen). The reactions were processed using a QuantStudio 6 Flex Real-time PCR machine (Life Technologies) using miScript SYBR Green PCR Kit (Qiagen) under the following cycling steps: 15 min at 95° C.; 40 cycles at 95° C. for 15 s, 55° C. for 30 s, 70° C. for 30 s; melting curve from 95° C. to 60° C. at 1.6° C. s⁻¹Three technical repeats for each biological replicate (at least 3) were carried out. MiR-34a copy number was determined using a standard curve covering 1×10⁸ copies to 1×10³ copies.

### Statistics

For two-group analysis a two-tailed Student's t-test was used to examine group differences. Two-way or one-way analysis of variance (ANOVA) with post hoc Bonferroni correction was used for multigroup comparison using Prism statistical package (version 7, GraphPad Software). Error bars represent either mean±s.d. or mean±s.e.m. as denoted in the figure legends. Statistically significant P values are indicated in figures and/or legends as ***, P<0.005; **, P<0.01; *, P<0.05.

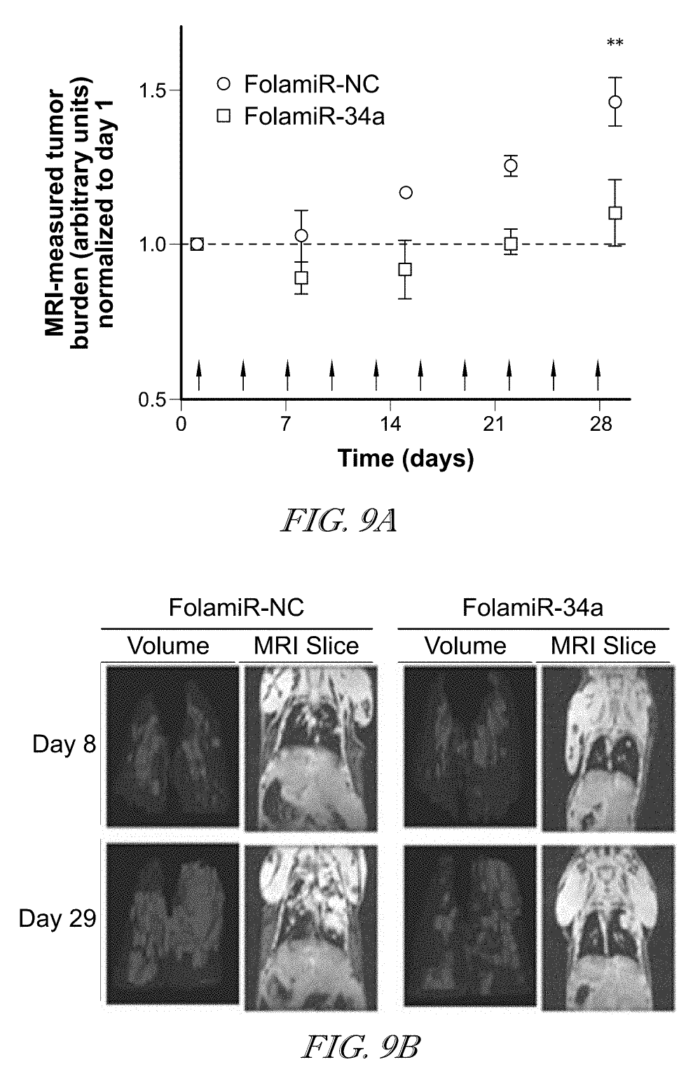

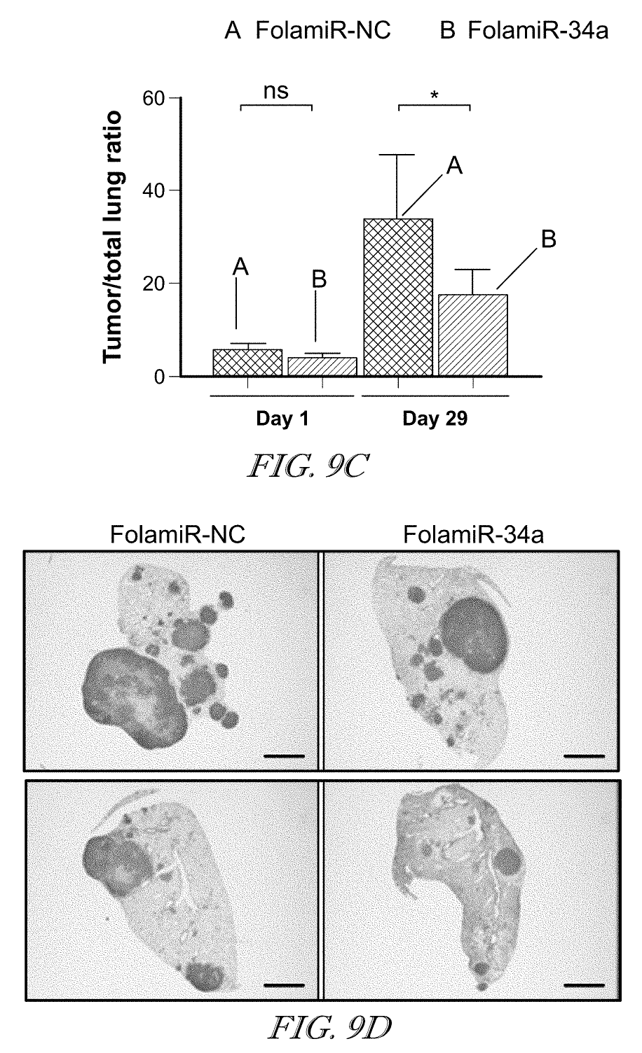

To determine if the KrasLSL-G12D/+; Trp53flx/flx tumors were responsive to FolamiR-34a, tumor bearing animals were intravenously administered 1 nmole of FolamiR-34a every three days for a total of ten doses. Tumor growth was monitored by MRI during the course of the study and the resulting volume measurements generated from the MRI data indicate that FolamiR-34a reduces tumor growth compared to animals administered the control folate-conjugate (FIGS. 9A, B, C). Tumor size was statistically unchanged in animals administered FolamiR-34a while tumor size in FolamiR-NC treated mice increased 1.5-fold relative to the first day of dosing. A similar response was observed at the histological level upon termination of the study (FIGS. 9D, E). Quantification of tumor burden indicated a statistically significant, 1.8-fold reduction in tumor burden in lungs harvested from FolamiR-34a treated mice relative to FolamiR-NC treated mice. Similar to the miR-34a copy number increase observed in MD-MBA-231 xenografts, copy number of miR-34a in the excised lung tumor tissue was ˜3 fold higher than in the tumors extracted from mice administered the control (FIG. 13B). To validate that miR-34a was acting to repress endogenous target genes transcript levels of the miR-34a targets, BCL-2, MET, and MYC were quantified. Both BCL-2 and MYC were statistically downregulated in tumors harvested from mice administered FolamiR-34a, confirming miR-34a activity on endogenous target genes (FIG. 9G).

1. A conjugate, or a pharmaceutically acceptable salt thereof, comprising:
a ligand (B) targeted to a cell-surface receptor;
one or more linkers (L);
one or more ionophores (A) each of which couples efflux of protons (H⁺ ions) to influx of potassium ions (K⁺ ions); and/or a therapeutic agent (TA) comprising an siRNA, an iRNA, or a microRNA;
wherein (L) optionally comprises at least one releasable linker; (B) is covalently linked to (L); and each of (A) and/or (TA) is covalently linked to (L).

2. The conjugate of claim 1, or a pharmaceutically acceptable salt thereof, wherein (L) comprises at least one releasable linker.

3. The conjugate of claim 1, or a pharmaceutically acceptable salt thereof, wherein the therapeutic agent (TA) is covalently linked to (L).

4. The conjugate of claim 1, or a pharmaceutically acceptable salt thereof, wherein the therapeutic agent (TA) comprises an siRNA.

5. The conjugate of claim 1, or a pharmaceutically acceptable salt thereof, wherein the therapeutic agent (TA) comprises an iRNA.

6. The conjugate of claim 1, or a pharmaceutically acceptable salt thereof, wherein the therapeutic agent (TA) comprises a microRNA.

7. The conjugate of claim 1, or a pharmaceutically acceptable salt thereof, wherein (B) is a folate or PSMA binding ligand.

8. The conjugate of claim 1, or a pharmaceutically acceptable salt thereof, wherein (A) is an inhibitor of the Na⁺/H⁺ exchanger.

9. The conjugate of claim 1, or a pharmaceutically acceptable salt thereof, wherein the ionophore (A) comprises nigericin or salinomycin.

10. The conjugate of claim 1, or a pharmaceutically acceptable salt thereof, wherein (L) comprises a chain of about 7 to about 45 atoms.

11. The conjugate of claim 1, having a formula selected from the group consisting of 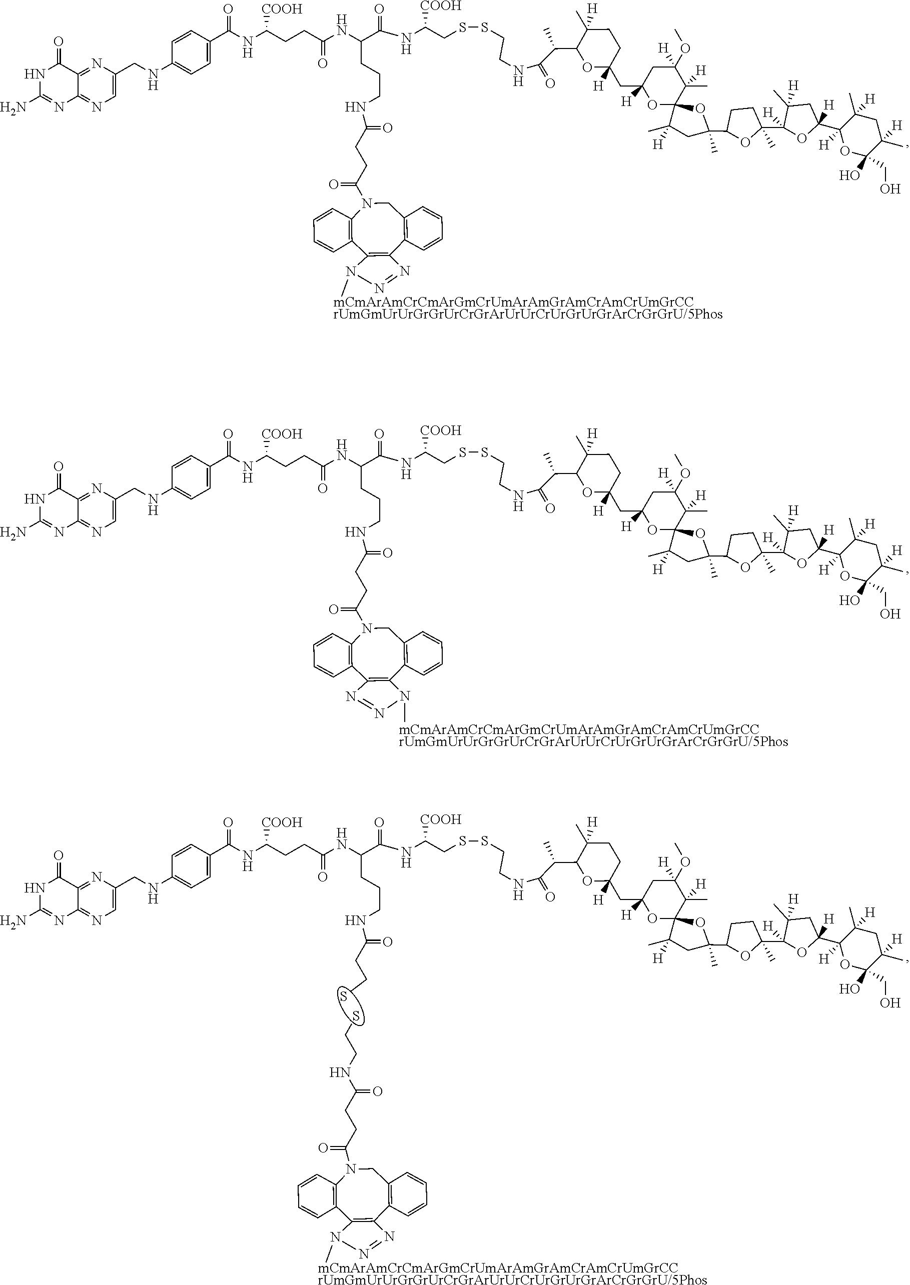 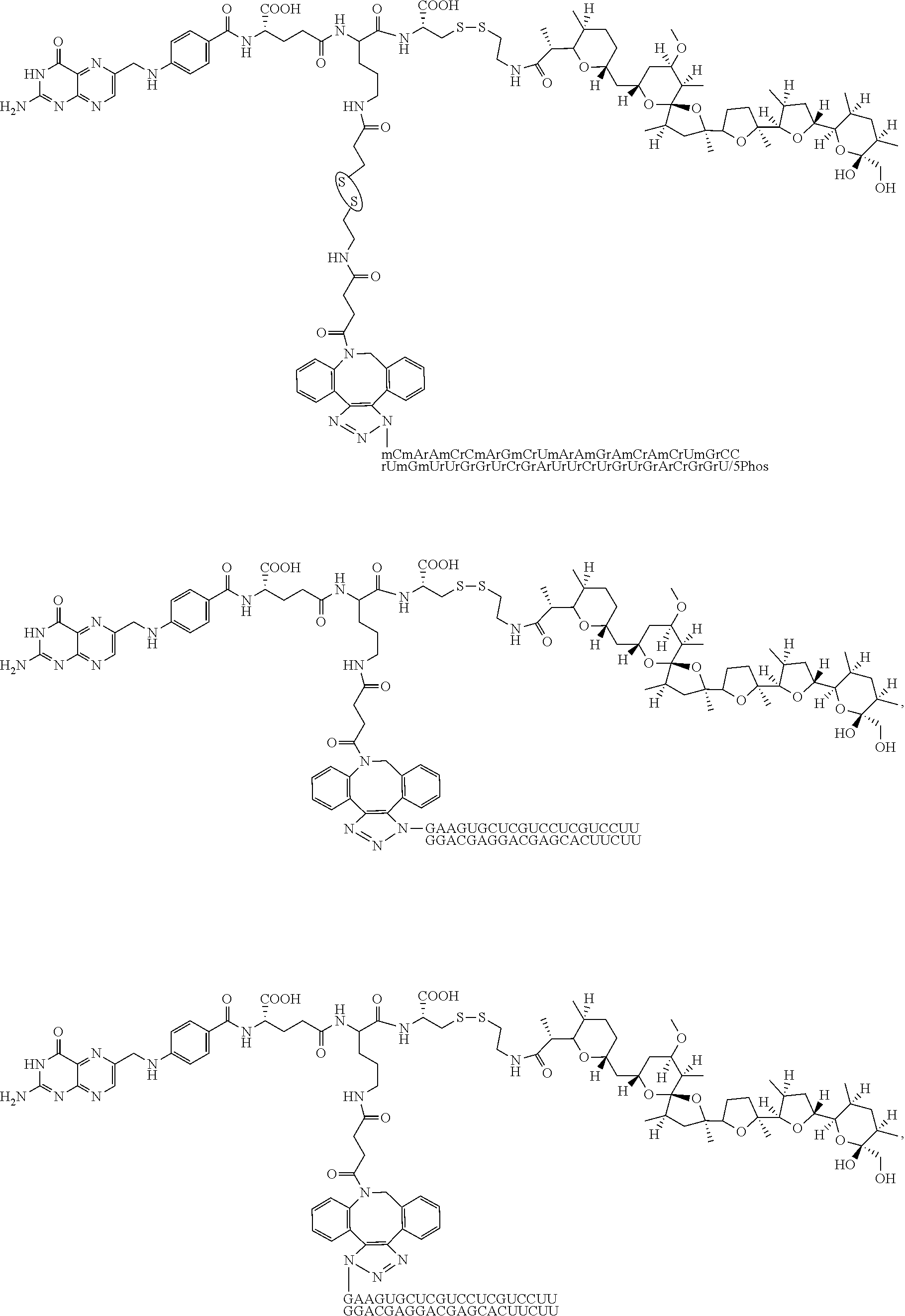 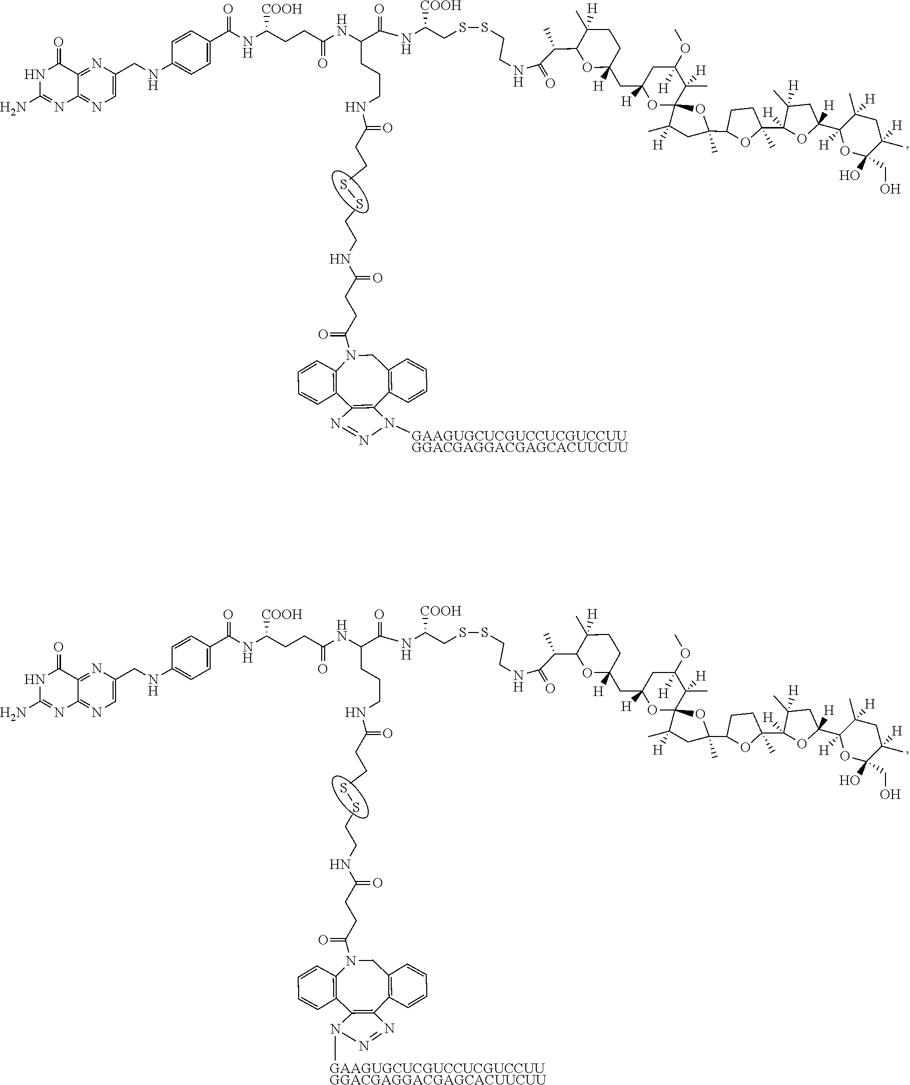 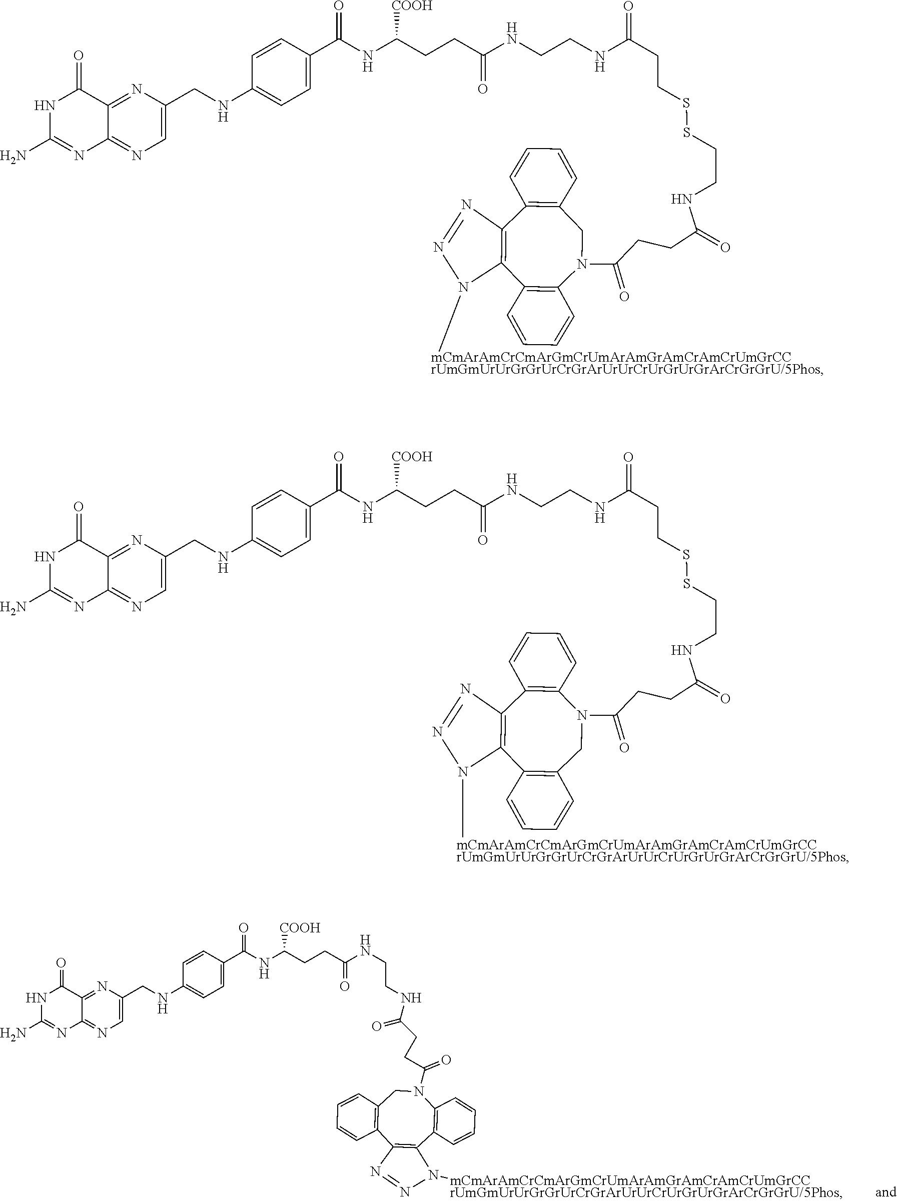 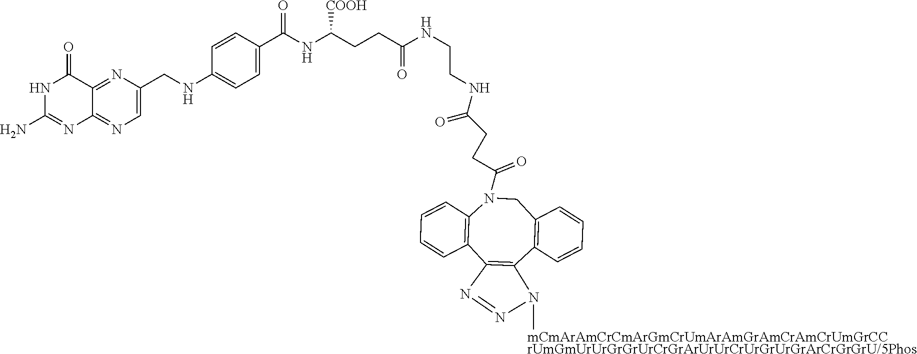
or a pharmaceutically acceptable salt thereof.

12. A pharmaceutical composition comprising at least one conjugate of claim 1, or a pharmaceutically acceptable salt thereof, and at least one pharmaceutically acceptable carrier or excipient.

13. A pharmaceutical composition comprising at least one conjugate of claim 1, or a pharmaceutically acceptable salt thereof, and an additional therapeutic agent.

14. A method of increasing the endosomal accumulation and escape of a therapeutic agent or an imaging agent, the method comprising the step of administering with the therapeutic agent or the imaging agent an effective amount of the conjugate of claim 1, or a pharmaceutically acceptable salt thereof.

15. The method of claim 14, wherein the therapeutic agent or the imaging agent is targeted to a cancer.

16. The method of claim 15, wherein the cancer is selected from the group consisting of ovarian, lung, breast, endometrial, brain, kidney, prostate, and colon cancer.

17. The method of claim 14, wherein the therapeutic agent is targeted to a site of inflammation.

18. The method of claim 17, wherein the site of inflammation is caused by an inflammatory disease selected from the group consisting of rheumatoid arthritis, osteoarthritis, atherosclerosis, diabetes, graft-versus-host disease, multiple sclerosis, osteomyelitis, psoriasis, Crohn's disease, Sjögren's syndrome, lupus erythematosus, and ulcerative colitis.

19. A conjugate, or a pharmaceutically acceptable salt thereof, comprising:
a ligand (B) targeted to a cell-surface receptor;
one or more linkers (L);
one or more of an ionophore (A) which couples efflux of protons (H⁺ ions) to influx of potassium ions (K⁺ ions); an RNA selected from an siRNA, an iRNA, and a microRNA; or an imaging agent (IA);
wherein (L) comprises at least one releasable linker; (B) is covalently linked to (L);
and each of (A), the RNA and/or (IA) is covalently linked to (L).

20. The conjugate of claim 21, having a formula 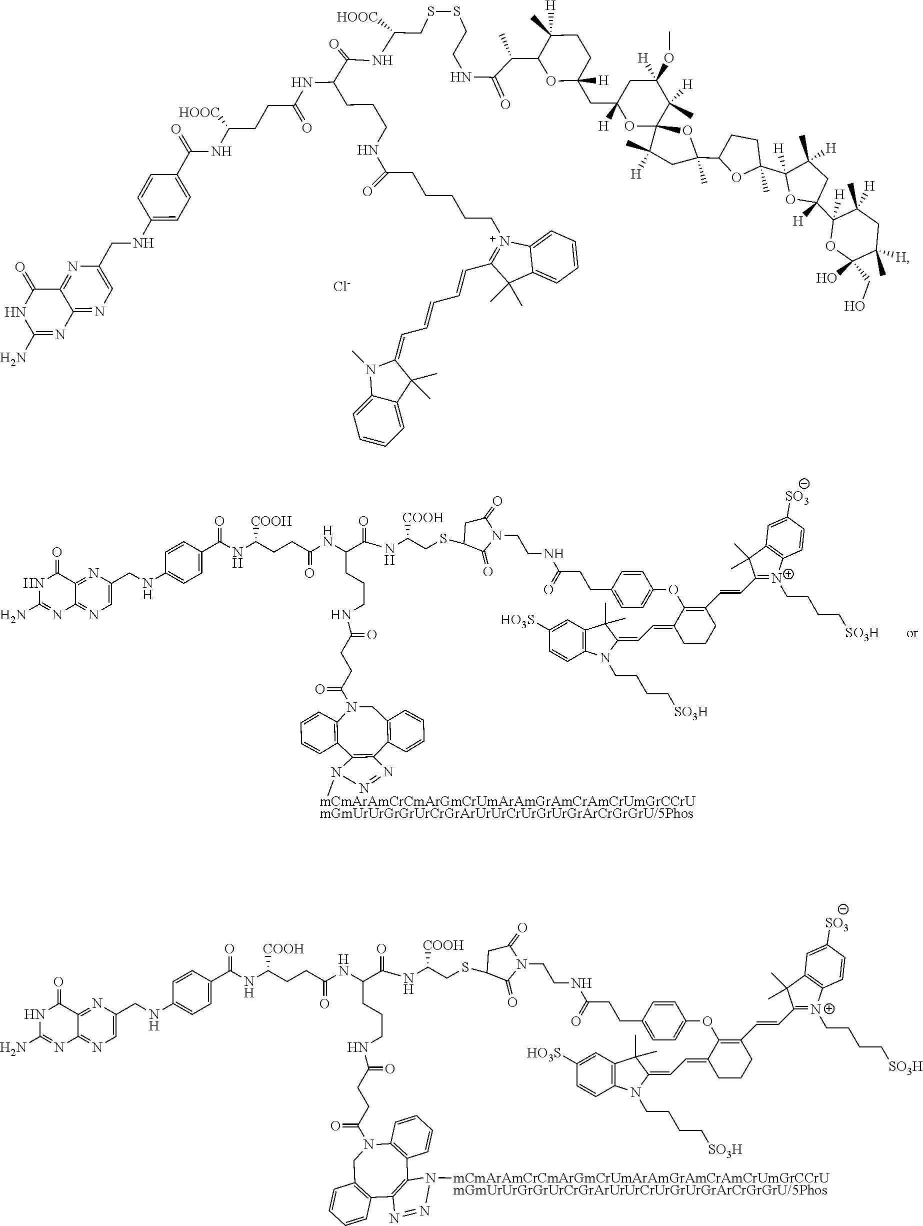
or a pharmaceutically acceptable salt thereof.
